SEQUENCE LISTING (30 sequences)

SEQ ID NO: 1 - protein, 16 aa, Artificial Sequence
  Synthetic polypeptide
GSLDESFYDWFERQLG

SEQ ID NO: 2 - protein, 16 aa, Artificial Sequence
  Synthetic polypeptide
XSLDXSFYDWFERQLG

SEQ ID NO: 3 - protein, 16 aa, Artificial Sequence
  Synthetic polypeptide
GXLDEXFYDWFERQLG

SEQ ID NO: 4 - protein, 16 aa, Artificial Sequence
  Synthetic polypeptide
GSXDESXYDWFERQLG

SEQ ID NO: 5 - protein, 16 aa, Artificial Sequence
  Synthetic polypeptide
GSLXESFXDWFERQLG

SEQ ID NO: 6 - protein, 16 aa, Artificial Sequence
  Synthetic polypeptide
GSLDXSFYXWFERQLG

SEQ ID NO: 7 - protein, 16 aa, Artificial Sequence
  Synthetic polypeptide
GSLDEXFYDXFERQLG

SEQ ID NO: 8 - protein, 16 aa, Artificial Sequence
  Synthetic polypeptide
GSLDESXYDWXERQLG

SEQ ID NO: 9 - protein, 16 aa, Artificial Sequence
  Synthetic polypeptide
GSLDESFXDWFXRQLG

SEQ ID NO: 10 - protein, 16 aa, Artificial Sequence
  Synthetic polypeptide
GSLDESFYXWFEXQLG

SEQ ID NO: 11 - protein, 16 aa, Artificial Sequence
  Synthetic polypeptide
GSLDESFYDXFERXLG

SEQ ID NO: 12 - protein, 16 aa, Artificial Sequence
  Synthetic polypeptide
GSLDESFYDWXERQXG

SEQ ID NO: 13 - protein, 16 aa, Artificial Sequence
  Synthetic polypeptide
GSLDESFYDWFXRQLX

SEQ ID NO: 14 - protein, 18 aa, Artificial Sequence
  Synthetic polypeptide
LKELEESSFRKTFEDYLH

SEQ ID NO: 15 - protein, 16 aa, Artificial Sequence
  Synthetic polypeptide
GSLDESFYDWFERQLG

SEQ ID NO: 16 - protein, 16 aa, Artificial Sequence
  Synthetic polypeptide
XSLDXSFYDWFERQLG

SEQ ID NO: 17 - protein, 16 aa, Artificial Sequence
  Synthetic polypeptide
GXLDEXFYDWFERQLG

SEQ ID NO: 18 - protein, 16 aa, Artificial Sequence
  Synthetic polypeptide
GSLDXSFYXWFERQLG

SEQ ID NO: 19 - protein, 16 aa, Artificial Sequence
  Synthetic polypeptide
GSLDXSFYDWFXRQLG

SEQ ID NO: 20 - protein, 16 aa, Artificial Sequence
  Synthetic polypeptide
GSLDEXFYDWFEXQLG

SEQ ID NO: 21 - protein, 16 aa, Artificial Sequence
  Synthetic polypeptide
XSLDXSFYXWFERQLG

SEQ ID NO: 22 - protein, 16 aa, Artificial Sequence
  Synthetic polypeptide
GXLDEXFYDWFEXQLG

SEQ ID NO: 23 - protein, 16 aa, Artificial Sequence
  Synthetic polypeptide
GSLDXSFYXWFERQLX

SEQ ID NO: 24 - protein, 16 aa, Artificial Sequence
  Synthetic polypeptide
GXLDESFYXWFERQLX

SEQ ID NO: 25 - protein, 16 aa, Artificial Sequence
  Synthetic polypeptide
ELEESSFRKTFEDYLH

SEQ ID NO: 26 - protein, 16 aa, Artificial Sequence
  Synthetic polypeptide
ELEESSFYKWFEDYLH

SEQ ID NO: 27 - protein, 16 aa, Artificial Sequence
  Synthetic polypeptide
ELEESSARKTAEDYLH

SEQ ID NO: 28 - protein, 16 aa, Artificial Sequence
  Synthetic polypeptide
ELEESSWRKTWEDYLH

SEQ ID NO: 29 - protein, 16 aa, Artificial Sequence
  Synthetic polypeptide
ELEESSDRKTDEDYLH

SEQ ID NO: 30 - protein, 16 aa, Artificial Sequence
  Synthetic polypeptide
ELEESSARKTAEDALH

(19) United States
(12) Patent Application Publication
(10) Pub. No.: US 2015/0225471 A1
(43) Pub. Date: Aug. 13, 2015

(54) STABILIZED POLYPEPTIDE INSULIN RECEPTOR MODULATORS

(71) Applicant: PRESIDENT AND FELLOW OF HARVARD COLLEGE, Cambridge, MA (US)

(72) Inventors: Rebecca Yue Liang, Cambridge, MA (US); Minyun Zhou, Malden, MA (US); Gregory L. Verdine, Boston, MA (US)

(73) Assignee: President and Fellows of Harvard College, Cambridge, MA (US)

(21) Appl. No.: 14/432,804

(22) PCT Filed: Oct. 1, 2013
(86) PCT No.: PCT/US2013/062929

§ 371 Date: Apr. 1, 2015

Related U.S. Application Data
(60) Provisional application No. 61/708,371, filed on Oct. 1, 2012.

(51) Int. Cl. C07K 14/72

(52) U.S. Cl. CPC C07K 14/72; A61K 38/00

(57) ABSTRACT

Provided herein are stapled or stitched polypeptides comprising an alpha-helical segment, wherein the polypeptide binds to the insulin receptor, and wherein the polypeptide comprises at least two cross-linked amino acids as shown in Formula (iii), or at least three cross-linked amino acids as shown in Formula (iv). Further provided are pharmaceutical compositions comprising the stapled or stitched polypeptides, methods of use, e.g., methods of treating a diabetic condition or complications thereof. Precursor “unstapled” polypeptides useful in the preparation of stapled and stitched polypeptides are also described.

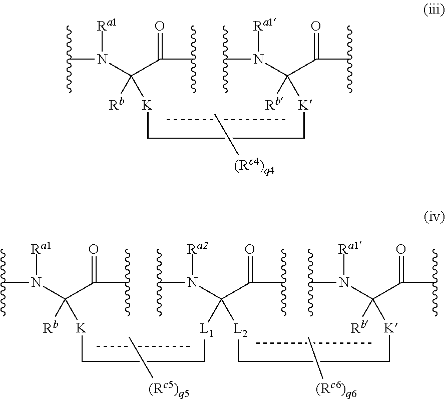

### RELATED APPLICATIONS

The present application claims priority under 35 U.S.C. §119(e) to U.S. provisional patent application, U.S. Ser. No. 61/708,371, filed Oct. 1, 2012, the entirety of which is incorporated herein by reference.

### BACKGROUND OF THE INVENTION

Insulin binding to the insulin receptor (IR) initiates a signaling cascade that plays an essential role in glucose homeostasis. Disruptions of this metabolic pathway may result in diabetes, a disease that afflicted 8.4% of the U.S. population in 2011. A key step toward combating diabetes is to understand ligand-dependent IR signaling and to develop new pharmacologic agents that modulate IR. Remarkably, despite extensive efforts spanning several decades, the molecular mechanisms of IR activation by the binding of insulin remain unelucidated.

IR, a member of the receptor tyrosine kinase superfamily, is a glycoprotein consisting of two α and two β subunits (α₂β₂) covalently linked by disulfide bonds. See, e.g., Siddle et al., Biochem Soc Trans (2001) 29:513-525. The extracellular domain (also called the ectodomain) of the IR comprises two α subunits and the N-terminal segment of the two β subunits, whereas the transmembrane domains and cytoplasmic tyrosine kinase (TK) domains comprise the C-terminal segments of the β subunits. The insulin-binding determinants reside entirely within the ectodomain, which consists of leucine-rich repeat domains L1 and L2 of the α chain, the intervening cysteine-rich (CR) domain, and three fibronectin type III domains, namely Fn0, Fn1, and Fn2.

Although insulin itself was the first peptide hormone to be structurally elucidated by X-ray crystallography, see, e.g., Blundell et al., Nature (1971) 231:506-511, and has been the subject of extensive structural investigations over the past 50 years, the molecular mechanism of IR activation by insulin remains unelucidated. No structure of insulin bound to the IR ectodomain has yet been determined, and while crystal structures of the unliganded IR ectodomain and the L1-CR-L2 ectodomain fragment have been solved, neither adopts a conformation having high-affinity insulin binding. See, e.g., McKern et al., Nature (2006) 443:218-221; Smith et al., Proc Natl Acad Sci USA (2010) 107:6771-6776; Lou et al., Proc Natl Acad Sci USA (2006) 103:12429-12434. Insulin binding to IR is characterized by exceptionally high-affinity binding (pM range) and negative cooperativity. See, e.g., De Meyts et al., Biochem Biophys Res Commun (1973) 55:154-161; De Meyts et al., Diabetologia (1994) 37 Suppl 2:S135-148. Evidence suggests that there are two insulin binding sites on the IR, site 1 and 2, wherein each site 1 on one monomer of IR is close to site 2′ on the second monomer, and binding of insulin to site 1 induces its subsequent binding to site 2′, which causes a conformational change of the IR ectodomain, leading to a reduction of the distance between the two intercellular TK domains, thereby facilitating autophosphorylation. See, e.g., FIG. 1. Several lines of evidence have shown that site 1 on the IR is formed by the central β-sheet of the L1 domain and a C terminal α-subunit peptide segment termed αCT (aa704-aa719), while site 2 is believed to reside at the loop region between Fn0 and Fn1, since it faces site 1 of the other monomer in the dimeric structure of the IR ectodomain. See, e.g., Huang et al., J Mol Biol (2004) 341:529-551; Mynarcik et al., J Biol Chem (1997) 272:18650-18651; Kurose et al., J Biol Chem (1994) 269:29190-29197; Mynarcik et al., J Biol Chem (1996) 271:2439-2442.

Peptides that bind site 1 are either agonists or antagonists, while peptides that bind site 2 are antagonists. Further optimization of site 1 and site 2 peptides by dimerization has identified either potent agonists or antagonists (pM IR binding affinity) depending on the mode of linkage. See, e.g., Schaffer et al., Proc Natl Acad Sci USA (2003) 100:4435-4439; Schaffer et al., Biochem Biophys Res Commun (2008) 376:380-383; Jensen et al., Biochem J (2008) 412:435-445. Intriguingly, though these peptides show no sequence similarity with insulin, a close relationship was proposed between the site 1 peptide and α-CT, indicating site 1 peptides are α-helical. See, e.g., Smith et al., Proc Natl Acad Sci USA (2010) 107:6771-6776; Menting et al., Biochemistry (2009) 48:5492-5500. Although these peptides are attractive candidates for insulin mimetics, the potential for therapeutic use is limited due to their inherent structural instability; therefore, there remains a need for stabilized peptides that bind the IR for therapeutic as well as scientific purposes.

### SUMMARY OF THE INVENTION

Peptide stapling and stitching is a synthetic strategy known to increase helix stabilization, in which adjacent or subsequent turns of an α-helix are cross-linked by an all-hydrocarbon macrocyclic bridge. See, e.g., Kim et al., Nat. Protoc. (2011) 6:761-771. This incorporated all-hydrocarbon staple can enforce the bioactive α-helical conformation of a synthetic peptide and confer on it increased target affinity, robust cell penetration, and/or extended in vivo half-life.

The present invention seeks to build from this knowledge of stapling and stitching a panel of stabilized (stapled or stitched) α-helical peptides that target the insulin receptor (IR), specifically the ectodomain of the IR, such as a stabilized polypeptide designed from the previously reported insulin-mimetic peptide 5371. Such stabilized peptides may have IR agonist or antagonist activity.

Thus, in one aspect, provided is a stabilized (stapled or stitched) polypeptide comprising an alpha-helical segment, wherein the polypeptide binds to the insulin receptor, and wherein the polypeptide comprises at least two cross-linked amino acids as shown in Formula (iii) (i.e., a stapled peptide):

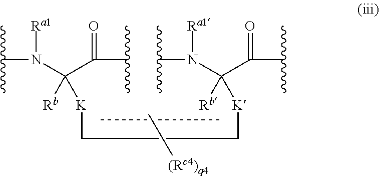

or at least three cross-linked amino acids as shown in Formula (iv) (i.e., a stitched peptide):

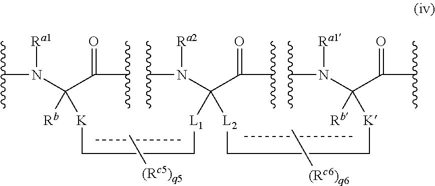

wherein:

each instance of K, K′, L₁, and L₂, is, independently a bond or a group consisting of a combination of one or more of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted heterocyclene; substituted and unsubstituted carbocyclene; substituted and unsubstituted arylene; and substituted and unsubstituted heteroarylene;

each instance of R^{a1}, R^{a1′}, and R^{a2} is, independently, hydrogen; substituted or unsubstituted aliphatic; substituted or unsubstituted heteroaliphatic; substituted or unsubstituted aryl; substituted or unsubstituted heteroaryl; acyl; or an amino protecting group;
each instance of R^{b} and R^{b′} is, independently, hydrogen; substituted or unsubstituted aliphatic; substituted or unsubstituted heteroaliphatic; substituted or unsubstituted aryl; or substituted or unsubstituted heteroaryl;
each instance of [symbol] independently represents a single or double bond;
each instance of R^{c4}, R^{c5}, and R^{c6} is independently hydrogen; substituted or unsubstituted aliphatic; substituted or unsubstituted heteroaliphatic; substituted or unsubstituted aryl; substituted or unsubstituted heteroaryl; acyl; substituted or unsubstituted hydroxyl; substituted or unsubstituted thiol; substituted or unsubstituted amino; azido; cyano; isocyano; halo; or nitro; and
each instance of q^{c4}, q^{c5}, and q^{c6} is independently 0, 1, or 2;
   or a pharmaceutically acceptable salt thereof.

In certain embodiments, the two cross-linked amino acids of Formula (iii) or the three cross-linked amino acids of Formula (iv) are amino acids of an alpha helical segment of the peptide. In certain embodiments, the alpha helical segment binds to the insulin receptor or contributes to the binding of the peptide to the insulin receptor.

In certain embodiments, the stabilized (stapled or stitched) peptide is of Formula (II):

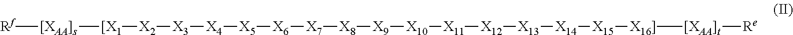

or a pharmaceutically acceptable salt thereof;
wherein:

each [X_{AA}] is independently a natural or unnatural amino acid;

s is 0 or an integer of between 1 and 50, inclusive;

t is 0 or an integer of between 1 and 50, inclusive;

R^{f} is an N-terminal group selected from the group consisting of hydrogen; substituted and unsubstituted aliphatic; substituted and unsubstituted heteroaliphatic; substituted and unsubstituted aryl; substituted and unsubstituted heteroaryl; acyl; a resin; an amino protecting group; and a label optionally joined by a linker, wherein the linker is a group consisting of a combination of one or more of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted arylene; substituted and unsubstituted heteroarylene; and acylene;

R^{e} is a C-terminal group selected from the group consisting of hydrogen; substituted and unsubstituted aliphatic; substituted and unsubstituted heteroaliphatic; substituted and unsubstituted aryl; substituted and unsubstituted heteroaryl; —OR^{E}; —N(R^{E})₂; and —SR^{E}; wherein each instance of R^{E} is, independently, hydrogen; substituted or unsubstituted aliphatic; substituted or unsubstituted heteroaliphatic; substituted or unsubstituted aryl; substituted or unsubstituted heteroaryl; acyl; a resin; a protecting group; or two R^{E} groups taken together form an substituted or unsubstituted heterocyclic or substituted or unsubstituted heteroaryl ring;

X₁ is amino acid G or is an amino acid which forms together with another amino acid a staple of Formula (iii);

X₂ is amino acid S or is an amino acid which forms together with another amino acid a staple of Formula (iii);

X₃ is amino acid L;

X₄ is amino acid D;

X₅ is amino acid E, is an amino acid which forms together with another amino acid a staple of Formula (iii), or is an amino acid which forms together with two other amino acids a stitch of formula (iv);

X₆ is amino acid S, is an amino acid which forms together with another amino acid a staple of Formula (iii), or is an amino acid which forms together with two other amino acids a stitch of formula (iv);

X₇ is amino acid F;

X₈ is amino acid Y;

X₉ is amino acid D or is an amino acid which forms together with another amino acid a staple of Formula (iii);

X₁₀ is amino acid W;

X₁₁ is amino acid F;

X₁₂ is amino acid E or is an amino acid which forms together with another amino acid a staple of Formula (iii);

X₁₃ is amino acid R or is an amino acid which forms together with another amino acid a staple of Formula (iii);

X₁₄ is amino acid Q;

X₁₅ is amino acid L; and

X₁₆ is amino acid G;

provided that the amino acid sequence comprises at least one staple of Formula (iii) or at least one stitch of Formula (iv).

In certain embodiments, the amino acid sequence comprises at least one staple of Formula (iii) at the i,i+3 position, i,i+4 position, or the i,i+7 position; or at least one stitch of Formula (iv) at the i,i+4+4 position, the i,i+3+4 position, the i,i+3+7 position, or the i,i+4+7 position.

In certain embodiments, one or more amino acids of the peptide of Formula (II) is mutated to another natural or unnatural amino acid. In certain embodiments, one, two, three, four, five, six, or more of X₁ through X₁₆ is mutated to another natural or unnatural amino acid.

In another aspect, provided is a precursor peptide comprising an alpha-helical segment, wherein the peptide binds to the insulin receptor either before and/or after stapling or stitching, and wherein the polypeptide comprises at least two amino acid moieties of Formula (i), and optionally, one amino acid of Formula (ii):

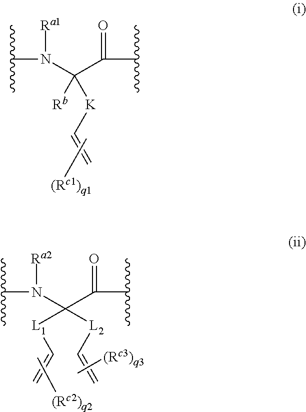

wherein:

each instance of K, L₁, and L₂, is, independently a bond or a group consisting of a combination of one or more of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted heterocyclene; substituted and unsubstituted carbocyclene; substituted and unsubstituted arylene; and substituted and unsubstituted heteroarylene;

each instance of R^{a1} and R^{a2} is, independently, hydrogen; substituted or unsubstituted aliphatic; substituted or unsubstituted heteroaliphatic; substituted or unsubstituted aryl; substituted or unsubstituted heteroaryl; acyl; or an amino protecting group;

R^{b} is hydrogen; substituted or unsubstituted aliphatic; substituted or unsubstituted heteroaliphatic; substituted or unsubstituted aryl; or substituted or unsubstituted heteroaryl;

each instance of R^{c1}, R^{c2}, and R^{c3} is independently hydrogen; substituted or unsubstituted aliphatic; substituted or unsubstituted heteroaliphatic; substituted or unsubstituted aryl; substituted or unsubstituted heteroaryl; acyl; substituted or unsubstituted hydroxyl; substituted or unsubstituted thiol; substituted or unsubstituted amino; azido; cyano; isocyano; halo; or nitro; and

each instance of q^{c1}, q^{c2}, and q^{c3} is independently 0, 1, or 2; or a pharmaceutically acceptable salt thereof.

In certain embodiments, the amino acids of Formula (i) and optionally Formula (ii) are amino acids of the alpha helical segment of the peptide. In certain embodiments, the alpha helical segment binds to or contributes to the binding of the peptide to the insulin receptor before and/or after stapling or stitching.

In certain embodiments, the precursor polypeptide is of Formula (I):

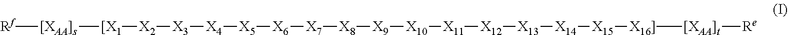

or a pharmaceutically acceptable salt thereof;
wherein:

each [X_{AA}] is independently a natural or unnatural amino acid;

s is 0 or an integer of between 1 and 50, inclusive;

t is 0 or an integer of between 1 and 50, inclusive;

R^{f} is an N-terminal group selected from the group consisting of hydrogen; substituted and unsubstituted aliphatic; substituted and unsubstituted heteroaliphatic; substituted and unsubstituted aryl; substituted and unsubstituted heteroaryl; acyl; a resin; an amino protecting group; and a label optionally joined by a linker, wherein the linker is a group consisting of a combination of one or more of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted arylene; substituted and unsubstituted heteroarylene; and acylene;

R^{e} is a C-terminal group selected from the group consisting of hydrogen; substituted and unsubstituted aliphatic; substituted and unsubstituted heteroaliphatic; substituted and unsubstituted aryl; substituted and unsubstituted heteroaryl; —OR^{E}; —N(R^{E})₂; and —SR^{E}, wherein each instance of R^{E} is, independently, hydrogen; substituted or unsubstituted aliphatic; substituted or unsubstituted heteroaliphatic; substituted or unsubstituted aryl; substituted or unsubstituted heteroaryl; acyl; a resin; or a protecting group; or two R^{E} groups taken together form an substituted or unsubstituted heterocyclic or substituted or unsubstituted heteroaryl ring;

X₁ is amino acid G or an amino acid of Formula (i);

X₂ is amino acid S or an amino acid of Formula (i);

X₃ is amino acid L;

X₄ is amino acid D;

X₅ is amino acid E, an amino acid of Formula (i), or an amino acid of Formula (ii);

X₆ is amino acid S, an amino acid of Formula (i), or an amino acid of Formula (ii);

X₇ is amino acid F;

X₈ is amino acid Y;

X₉ is amino acid D or an amino acid of Formula (i);

X₁₀ is amino acid W;

X₁₁ is amino acid F;

X₁₂ is amino acid E or an amino acid of Formula (i);

X₁₃ is amino acid R or an amino acid of Formula (i);

X₁₄ is amino acid Q;

X₁₅ is amino acid L; and

X₁₆ is amino acid G;

provided that the amino acid sequence comprises two independent occurrences of an amino acid of Formula (i), and/or one occurrence of Formula (ii).

In certain embodiments, the amino acid sequence comprises two independent occurrences of an amino acid of Formula (i) separated by two (i,i+3) amino acids, three (i,i+4) amino acids, or six (i,i+7) amino acids, and/or one occurrence of Formula (ii) and two amino acids of Formula (i) peripheral thereto each separated by three (i,i+4+4) amino acids, separated by two and three amino acids (i,i+3+4), separated by two and six amino acids (i,i+3+7), or separated by three and six (i,i+4+7) amino acids.

In certain embodiments, one or more amino acids of the peptide of Formula (I) is mutated to another natural or unnatural amino acid. In certain embodiments, one, two, three, four, five, six, or more of X₁ through X₁₆ is mutated to another natural or unnatural amino acid.

In another aspect, provided are pharmaceutical compositions comprising a stabilized (stapled or stitched) polypeptide as described herein, or a pharmaceutically acceptable salt thereof, and optionally a pharmaceutically acceptable excipient. The pharmaceutical composition may be useful in the treatment of diabetes or pre-diabetes.

In yet another aspect, provided are methods of treating a diabetic condition or a complication thereof comprising administering to a subject in need thereof an effective amount of a stabilized (stapled or stitched) polypeptide as described herein, or a pharmaceutically acceptable salt thereof. In certain embodiments, the diabetic condition is diabetes or pre-diabetes. In certain embodiments, the diabetes is Type I diabetes, Type 2 diabetes, gestational diabetes, congenital diabetes, cystic fibrosis-related diabetes, steroid diabetes, or monogenic diabetes. In certain embodiments, the complication of the diabetic condition is cardiovascular disease, ischemic heart disease, stroke, peripheral vascular disease, damage to blood vessels, diabetic retinopathy, diabetic nephropathy, chronic kidney disease, diabetic neuropathy, and diabetic foot ulcers.

### BRIEF DESCRIPTION OF THE DRAWINGS

FIGS. 1A and 1B depict a two-site model of IR activation. Two IR molecules are shown. FIG. 1A shows insulin binding sites on IR. Site 1 is formed by the central β-sheets of L1 and αCT, and site 2 is the loop between Fn0 and Fn1. FIG. 1B is a schematic representation of Insulin or peptide surrogates binding modes. Insulin binds to site 1 on one monomer and then the adjacent site 2′ on the other monomer, leading to conformational changes of IR. In contrast, insulin mimetic peptides (side 1 or 2 peptides) bind to either site 1 or site 2 only.

FIGS. 2A-2F depict a schematic representation of stapled insulin receptor binder (SIRB) peptides and an example of staple “scanning.” FIGS. 2A-2D depict four classes of hydrocarbon stapled peptides are made by incorporation of non-natural amino acids with olefinic side chains varying in length and stereochemistry: (A) i+4; (B) i+7; (C) i+4+4; and (D) i+4+7, named stapled insulin receptor binder (SIRB) series A-D, respectively. FIG. 2E also depicts six types of hydrocarbon crosslinks featuring single staple (i, i+3; i, i+4; i, i+7) and stitching (i, i+4+4; i, i+4+7, i, i+7+7). Spheres represent non-natural amino acids. To create a comprehensive library, all possible stapling positions were sampled in each series. FIG. 2F depicts an example of staple “scanning” through the S371 sequence for i, i+4 is also provided.

FIGS. 3A-3B depict SIRB (A) antagonism and (B) agonism, measured by an ELISA assay specific for Akt-S473 phosphorylation. (A) HepG2 cells were treated with 10 μM peptide in the presence of 50 nM insulin. Six SIRB peptides exhibited greater than a 60% reduction of the insulin-induced signal. (B) HepG2 cells were treated with 10 or 100 μM peptide in the absence of insulin. SIRB-D2 at 100 M exhibits agonism equal to approximately 60% of the insulin-induced signal. All readouts (in RLU) were normalized to the signal by 50 nM insulin alone with vehicle as baseline.

FIGS. 4A-4D depicts the characterization of SIRB peptides. FIGS. 4A-4C depict the effects of SIRB peptides on Phospho-IR level detected by western blot. CHO-IR cells were treated with 10 M peptides in the presence of 50 nM insulin. A significant reduction of auto-phosphorylation of IR is observed when treated with SIRB A2, A5, and B5 peptides. FIG. 4D depicts the results of CD spectroscopy of active SIRB peptides in each crosslink series illustrates an increase in helicity brought by synthetic modification.

FIG. 5A-5B depicts the hot spots for hydrocarbon staples. (A) The panel of active peptides derived from S371. Residues conserved between α-CT and S371 are boxed; non-natural residues forming the cross-links are shown circled. (B) The interaction model between S371 (top) and L1 domain (insulin binding site 1 on IR, bottom) of the IR. Staple hotspots on S371 are shown by the position of the spheres. Residues involved in direct interactions between S371 and the IR as well as residues in Fn0/Fn1 loop (insulin binding site 2 on IR, in wheat) that may interact with staples are shown in stick rendering.

FIG. 6 depicts the dose-response of antagonist SIRB-B5. CHO-IR cells were treated with 50 nM insulin and increasing concentrations of SIRB-B5. ELISA assays of phosphor-IR Y1150/1151 and phosphor-Akt S473 were performed with cell lysates.

FIG. 7 depicts IR ectodomain constructs. FIG. 7A depicts truncation or deletion of the IR ectodomain that have been reported to retain high affinity for insulin. Constructs with or without α-CT will be used for crystallization and binding assays in the future with SIRB peptides in parallel to examine whether α-CT affects the interaction of SIRB peptides with the IR. FIG. 7B depicts expression of IR constructs. HEK293 cells were stably transfected with various constructs; protein secreted into expression medium could be visualized by Western blot of their His5 tag.

FIG. 8 depicts the affinity pull-down of IR by SIRB-B5. 1% BSA added to binding buffer. FIG. 8A depicts the pull-down assay: Biotinylated SIRB-B5 could be immobilized on streptavidincoated beads and pull down IR from expression medium. Precipitated protein could be visualized by Western blot of His5 tag. FIG. 8B depicts L1-CR-L2 pulled down by SIRB-B5. FIG. 8C depicts mIR-Fn1-Ex10 pulled down by SIRB-B5 and competition by insulin.

FIG. 9 depicts the design of the competition pull-down experiment to probe the binding site of SIRB-B5. FIG. 9A depicts sequences of competitive peptides: wild-type αCT, gain-of-function double mutant, and loss-of function mutants are included as negative controls. FIG. 9B depicts the competitive pull-down experiment. FIG. 9C depicts a close-up view of interaction between αCT and L1 β-sheets. The two phenylalanine residues on αCT were chosen as mutation sites in the negative control peptides.

FIG. 10 depicts the results of a competitive pull-down assay. L1-CR-L2 was pulled down by biotinylated SIRB-B5 alone or in the presence of competitive peptides. FIG. 10A depicts the concentration ranges of αCTnm (loss-of-function negative control mutant), αCTwt (wild-type), and αCTdm (gain-of-function double mutant) (10, 50, and 100 M) evaluated in the competitive pull-down assay. FIG. 10B depicts the concentration ranges of αCTnm, αCTwt (10, 50, and 100 M), and αCTdm (0.1, 1, and 10 M) evaluated in the competitive pull-down assay, while increasing exposure duration.

FIG. 11 depicts SIRB-B5 antagonism on IGF-1R. HepG2 cells were treated with SIRB-B5 in the presence of IGF1. IGF-1R was immunoprecipitated to be distinguished from IR on a Phospho-IR/IGF-1R Western blot.

FIG. 12 depicts the SIRB homodimer synthesis. The free N-terminus of SIRB reacted readily with succinimide-containing linkers. Self-dimerization could be achieved in one step using a selection of bis(succinimide) linkers varying in the spacer arm length.

FIG. 13 depicts the increase of agonist potency by dimerization of SIRB-D2. CHO-IR cells were treated with increasing concentration of (D2)2Glu (1, 10, and 100 M) in the absence of insulin. Results are shown in comparison of stimulation by 50 nM insulin. Total IR levels were measured as control.

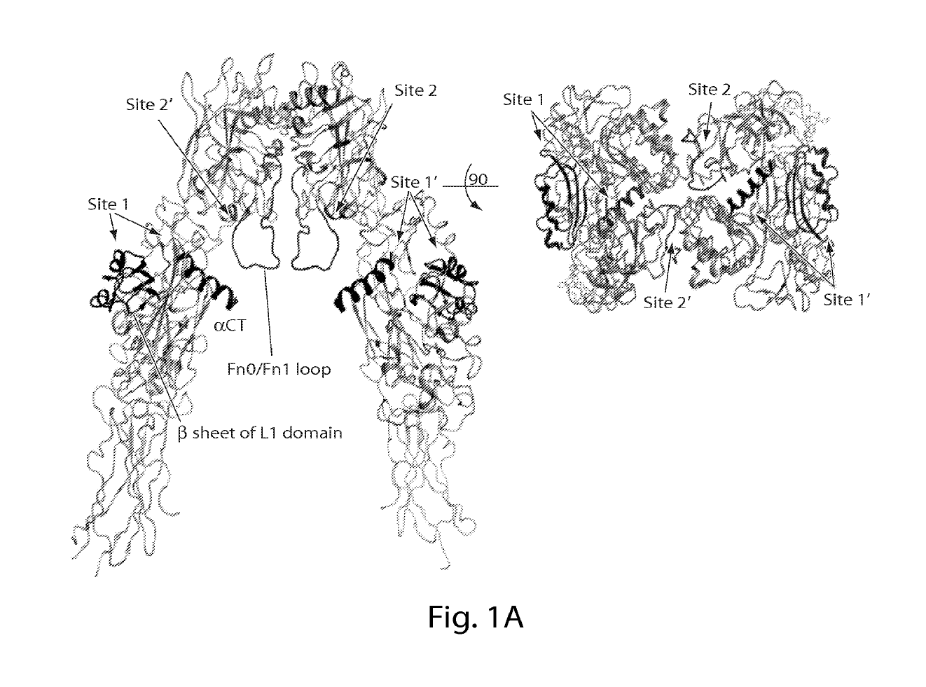

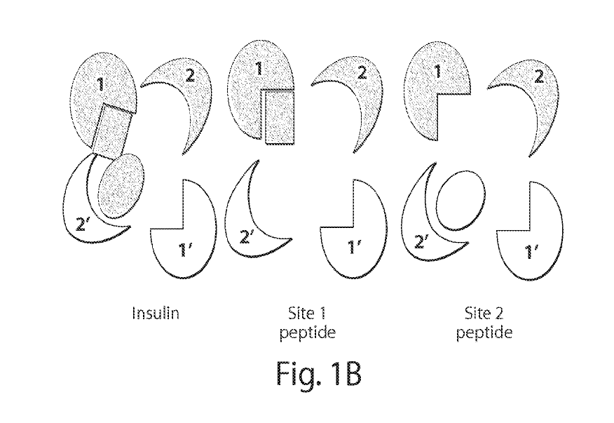

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987.

The compounds of the present invention (e.g., amino acids, and unstapled, partially stapled, and stapled polypeptides) may exist in particular geometric or stereoisomeric forms. The present invention contemplates all such compounds, including cis- and trans-isomers, R- and S-enantiomers, diastereomers, (D)- and (L)-isomers, the racemic mixtures thereof, and other mixtures thereof, as falling within the scope of the invention.

Where an isomer/enantiomer is preferred, it may, in some embodiments, be provided substantially free of the corresponding enantiomer, and may also be referred to as “optically enriched.” “Optically enriched,” as used herein, means that the compound is made up of a significantly greater proportion of one enantiomer. In certain embodiments, the compound of the present invention is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); Wilen, Tables of Resolving Agents and Optical Resolutions p. 268 (E. L. Eliel, Ed., Univ. of Notre Dame Press, Notre Dame, Ind. 1972).

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, substituent names which end in the suffix “-ene” refer to a biradical derived from the removal of an additional hydrogen atom from monoradical group as defined herein. Thus, for example, the monoradical alkyl, as defined herein, is the biradical alkylene upon removal of an additional hydrogen atom. Likewise, alkenyl is alkenylene; alkynyl is alkynylene; heteroalkyl is heteroalkylene; heteroalkenyl is heteroalkenylene; heteroalkynyl is heteroalkynylene; carbocyclyl is carbocyclylene; heterocyclyl is heterocyclylene; aryl is arylene; and heteroaryl is heteroarylene.

The term “aliphatic,” as used herein, refers to alkyl, alkenyl, alkynyl, and carbocyclic groups. Likewise, the term “heteroaliphatic” as used herein, refers to heteroalkyl, heteroalkenyl, heteroalkynyl, and heterocyclic groups.

As used herein, “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀ alkyl (e.g., —CH₃). In certain embodiments, the alkyl group is a substituted C₁₀ alkyl.

As used herein, “haloalkyl” is a substituted alkyl group as defined herein wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

As used herein, “heteroalkyl” refers to an alkyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1, 2, 3, or 4 heteroatoms within the parent chain (“heteroC₁₋₁₀ alkyl”). In some embodiments, a heteroalkyl group is a saturated group having 1 to 9 carbon atoms and 1, 2, 3, or 4 heteroatoms within the parent chain (“heteroC₁₋₉ alkyl”). In some embodiments, a heteroalkyl group is a saturated group having 1 to 8 carbon atoms and 1, 2, 3, or 4 heteroatoms within the parent chain (“heteroC₁₋₈ alkyl”). In some embodiments, a heteroalkyl group is a saturated group having 1 to 7 carbon atoms and 1, 2, 3, or 4 heteroatoms within the parent chain (“heteroC₁₋₇ alkyl”). In some embodiments, a heteroalkyl group is a saturated group having 1 to 6 carbon atoms and 1, 2, or 3 heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

As used herein, “alkenyl” refers to a radical of a straight-chain or branched hydrocarbon group having from 2 to 10 carbon atoms and one or more double bonds (e.g., 1, 2, 3, or 4 double bonds) and no triple bonds. In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl.

As used herein, “heteroalkenyl” refers to an alkenyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1, 2, 3, or 4 heteroatoms within the parent chain (“heteroC₂₋₁₀ alkenyl”). In some embodiments, a heteroalkenyl group has 2 to 9 carbon atoms at least one double bond, and 1, 2, 3, or 4 heteroatoms within the parent chain (“heteroC₂₋₉ alkenyl”). In some embodiments, a heteroalkenyl group has 2 to 8 carbon atoms, at least one double bond, and 1, 2, 3, or 4 heteroatoms within the parent chain (“heteroC₂₋₈ alkenyl”). In some embodiments, a heteroalkenyl group has 2 to 7 carbon atoms, at least one double bond, and 1, 2, 3, or 4 heteroatoms within the parent chain (“heteroC₂₋₇ alkenyl”). In some embodiments, a heteroalkenyl group has 2 to 6 carbon atoms, at least one double bond, and 1, 2, or 3 heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

As used herein, “alkynyl” refers to a radical of a straight-chain or branched hydrocarbon group having from 2 to 10 carbon atoms and one or more triple bonds (e.g., 1, 2, 3, or 4 triple bonds) and optionally one or more double bonds (e.g., 1, 2, 3, or 4 double bonds) (“C₂₋₁₀ alkynyl”). An alkynyl group that has one or more triple bonds and one or more double bonds is also referred to as an “ene-yene” group. In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

As used herein, “heteroalkynyl” refers to an alkynyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1, 2, 3, or 4 heteroatoms within the parent chain (“heteroC₂₋₁₀ alkynyl”). In some embodiments, a heteroalkynyl group has 2 to 9 carbon atoms, at least one triple bond, and 1, 2, 3, or 4 heteroatoms within the parent chain (“heteroC₂₋₉ alkynyl”). In some embodiments, a heteroalkynyl group has 2 to 8 carbon atoms, at least one triple bond, and 1, 2, 3, or 4 heteroatoms within the parent chain (“heteroC₂₋₈ alkynyl”). In some embodiments, a heteroalkynyl group has 2 to 7 carbon atoms, at least one triple bond, and 1, 2, 3, or 4 heteroatoms within the parent chain (“heteroC₂₋₇ alkynyl”). In some embodiments, a heteroalkynyl group has 2 to 6 carbon atoms, at least one triple bond, and 1, 2, or 3 heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₄ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₀ cycloalkyl. In certain embodiments, the cycloalkyl group is a substituted C₃₋₁₀ cycloalkyl.

As used herein, “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

As used herein, “aryl” refers to a radical of a monocyclic or polycyclic (e.g., bicyclic or tricyclic) 4n+2 aromatic ring system (e.g., having 6, 10, or 14 it electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group, as defined herein, substituted by an aryl group, as defined herein, wherein the point of attachment is on the alkyl moiety.

As used herein, “heteroaryl” refers to a radical of a 5-14 membered monocyclic or polycyclic (e.g., bicyclic or tricyclic) 4n+2 aromatic ring system (e.g., having 6, 10, or 14 it electrons shared in a cyclic array) having ring carbon atoms and 1-4 ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group, as defined herein, substituted by a heteroaryl group, as defined herein, wherein the point of attachment is on the alkyl moiety.

As used herein, the term “partially unsaturated” refers to a group that includes at least one double or triple bond. The term “partially unsaturated” is intended to encompass rings having multiple sites of unsaturation, but is not intended to include aromatic groups (e.g., aryl or heteroaryl moieties) as herein defined.

As used herein, the term “saturated” refers to a group that does not contain a double or triple bond, i.e., contains all single bonds.

As understood from the above, alkyl, alkenyl, alkynyl, heteroalkyl, heteroalkenyl, heteroalkynyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂Ra^{aa}, —C(═O)N(R^{bb})₂, —OC(═O)N(R^{bb})₂, —NR^{bb}C(═O)R^{aa}, —NR^{bb}CO₂R^{aa}, NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₄ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

As used herein, the term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

As used herein, the term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa} and R^{cc} are as defined herein.

As used herein, the term, “amino” refers to the group —NH₂. The term “substituted amino,” by extension, refers to a monosubstituted amino, a disubstituted amino, or a trisubstituted amino, as defined herein.

As used herein, the term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb}, and R^{cc} are as defined herein, and wherein R^{bb} of the group —NH(R^{bb}) is not hydrogen.

As used herein, the term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R, —NR^{bb}P(═O)(OR^{cc})₂, and —NR^{bb}P(═O)(NR^{bb})₂, wherein R^{aa}, R^{bb}, and R^{cc} are as defined herein, with the proviso that the nitrogen atom directly attached to the parent molecule is not substituted with hydrogen.

As used herein, the term “trisubstituted amino” or a “quaternary amino salt” or a “quaternary salt” refers to a nitrogen atom covalently attached to four groups such that the nitrogen is cationic, wherein the cationic nitrogen atom is further complexed with an anionic counterion, e.g., such as groups of the Formula —N(R^{bb})₃⁺X⁻ and —N(R^{bb})₂—⁺X⁻, wherein R^{bb} and X⁻ are as defined herein.

As used herein, a “counterion” or “anionic counterion” is a negatively charged group associated with a cationic quaternary amino group in order to maintain electronic neutrality. Exemplary counterions include halide ions (e.g., F⁻, Cl⁻, Br⁻, F⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

As used herein, the term “sulfonyl” refers to a group selected from —SO₂N(R^{bb})₂, —SO₂Ra^{aa}, and —SO₂OR^{aa}, wherein R^{aa} and R^{bb} are as defined herein.

As used herein, the term “sulfinyl” refers to the group —S(═O)R^{aa}, wherein R^{aa} is as defined herein.

As used herein, the term “acyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}), thioesters (—C(═O)SR^{aa}, —C(═S)SR^{aa}), amides (—C(═O)N(R^{bb})₂, —C(═O)NR^{bb}SO₂R^{aa}) thioamides (—C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

As used herein, the term “azido” refers to a group of the formula —N₃.

As used herein, the term “cyano” refers to a group of the formula —CN.

As used herein, the term “isocyano” refers to a group of the formula —NC.

As used herein, the term “nitro” refers to a group of the formula —NO₂.

As used herein, the term “halo” or “halogen” refers to fluorine (fluoro, —F), chlorine (chloro, —Cl), bromine (bromo, —Br), or iodine (iodo, —I).

As used herein, the term “oxo” refers to a group of the formula ═O.

As used herein, the term “thiooxo” refers to a group of the formula ═S.

As used herein, the term “imino” refers to a group of the formula ═N(R^{b}).

As used herein, the term “silyl” refers to the group —Si(R^{aa})₃, wherein R^{aa} is as defined herein.

Nitrogen atoms can be substituted or unsubstituted as valency permits, and include primary, secondary, tertiary, and quaternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an amino protecting group (also referred to herein as a “nitrogen protecting group”). Amino protecting groups include, but are not limited to, —OH, —OR, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Amino protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, amino protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Amino protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Amino protecting groups such as sulfonamide groups (e.g., —S(═O)₂Ra^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other amino protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is a hydroxyl protecting group (also referred to herein as an “oxygen protecting group”). Hydroxyl protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂Ra^{aa}, —Si(R^{aa})₃, —P(R^{cc})₂, —P(R^{cc})₃, —P(═O)₂R, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Hydroxyl protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary hydroxyl protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxyl)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

A “thiol protecting group” is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of protected thiol groups further include, but are not limited to, thioesters, carbonates, sulfonates allyl thioethers, thioethers, silyl thioethers, alkyl thioethers, arylalkyl thioethers, and alkyloxyalkyl thioethers. Examples of ester groups include formates, acetates, proprionates, pentanoates, crotonates, and benzoates. Specific examples of ester groups include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benylbenzoate, 2,4,6-trimethylbenzoate. Examples of carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of silyl groups include trimethylsilyl, triethylsilyl, t-butyldimethylsilyl, t-butyldiphenylsilyl, triisopropylsilyl ether, and other trialkylsilyl ethers. Examples of alkyl groups include methyl, benzyl, p-methoxybenzyl, 3,4-dimethoxybenzyl, trityl, t-butyl, and allyl ether, or derivatives thereof. Examples of arylalkyl groups include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

The term “amino acid” refers to a molecule containing both an amino group and a carboxyl group (e.g., carboxylic acid). Amino acids include alpha-amino acids and beta-amino acids, the structures of which are depicted below. In certain embodiments, the amino acid is an alpha-amino acid. In certain embodiments, the amino acid is a beta-amino acid. In certain embodiments, the amino acid is an unnatural amino acid. In certain embodiments, the amino acid is a natural amino acid. In certain embodiments, the amino acid is an unnatural amino acid.

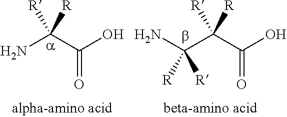

Exemplary amino acids include, without limitation, natural alpha amino acids such as the 20 common naturally occurring alpha amino acids found in peptides (e.g., A, R, N, C, D, Q, E, G, H, I, L, K, M, F, P, S, T, W, Y, V, as provided in Table 1 depicted below), unnatural alpha-amino acids (as depicted in Tables 2 and 3 below), natural beta-amino acids (e.g., beta-alanine), and unnatural beta-amino acids.

Amino acids used in the construction of peptides of the present invention may be prepared by organic synthesis, or obtained by other routes, such as, for example, degradation of protein or peptides, or isolation from a natural source. In certain embodiments of the present invention, each instance of the formula —[X_{AA}]— corresponds to an natural or unnatural amino acid of the formula:

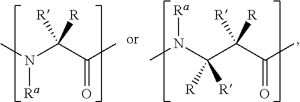

wherein R and R′correspond to an amino acid side chain, as defined below and herein, and wherein R^{a} is hydrogen; substituted or unsubstituted aliphatic; substituted or unsubstituted heteroaliphatic; substituted or unsubstituted aryl; substituted or unsubstituted heteroaryl; acyl; or an amino protecting group.

TABLE 1

| | |
|---|---|
| Exemplary natural | Amino acid side chains |

| alpha-amino acids | R | R′ |
|---|---|---|
| L-Alanine (A) | —CH₃ | —H |
| L-Arginine (R) | —CH₂CH₂CH₂—NHC(═NH)NH₂ | —H |
| L-Asparagine (N) | —CH₂C(═O)NH₂ | —H |
| L-Aspartic acid (D) | —CH₂CO₂H | —H |
| L-Cysteine (C) | —CH₂SH | —H |
| L-Glutamic acid (E) | —CH₂CH₂CO₂H | —H |
| L-Glutamine (Q) | —CH₂CH₂C(═O)NH₂ | —H |
| Glycine (G) | —H | —H |
| L-Histidine (H) | —CH₂-2-(1H-imidazole) | —H |
| L-Isoleucine (I) | -sec-butyl | —H |
| L-Leucine (L) | -iso-butyl | —H |
| L-Lysine (K) | —CH₂CH₂CH₂CH₂NH₂ | —H |
| L-Methionine (M) | —CH₂CH₂SCH₃ | —H |
| L-Phenylalanine (F) | —CH₂Ph | —H |
| L-Proline (P) | -2-(pyrrolidine) | —H |
| L-Serine (S) | —CH₂OH | —H |
| L-Threonine (T) | —CH₂CH(OH)(CH₃) | —H |
| L-Tryptophan (W) | —CH₂-3-(1H-indole) | —H |
| L-Tyrosine (Y) | —CH₂-(p-hydroxyphenyl) | —H |
| L-Valine (V) | -isopropyl | —H |

TABLE 2

| Exemplary unnatural | Amino acid side chains | |
|---|---|---|
| alpha-amino acids | R | R′ |
| D-Alanine | —H | —CH₃ |
| D-Arginine | —H | —CH₂CH₂CH₂—NHC(═NH)NH₂ |
| D-Asparagine | —H | —CH₂C(═O)NH₂ |
| D-Aspartic acid | —H | —CH₂CO₂H |
| D-Cysteine | —H | —CH₂SH |
| D-Glutamic acid | —H | —CH₂CH₂CO₂H |
| D-Glutamine | —H | —CH₂CH₂C(═O)NH₂ |
| D-Histidine | —H | —CH₂-2-(1H-imidazole) |
| D-Isoleucine | —H | -sec-butyl |
| D-Leucine | —H | -iso-butyl |
| D-Lysine | —H | —CH₂CH₂CH₂CH₂NH₂ |
| D-Methionine | —H | —CH₂CH₂SCH₃ |
| D-Phenylalanine | —H | —CH₂Ph |
| D-Proline | —H | -2-(pyrrolidine) |
| D-Serine | —H | —CH₂OH |
| D-Threonine | —H | —CH₂CH(OH)(CH₃) |
| D-Tryptophan | —H | —CH₂-3-(1H-indole) |
| D-Tyrosine | —H | —CH₂-(p-hydroxyphenyl) |
| D-Valine | —H | -isopropyl |
| Di-vinyl | —CH═CH₂ | —CH═CH₂ |
| Exemplary unnatural | | |
| alpha-amino acids | R and R′ are equal to: | |
| α-methyl-Alanine | —CH₃ | —CH₃ |
| (Aib) | | |
| α-methyl-Arginine | —CH₃ | —CH₂CH₂CH₂—NHC(═NH)NH₂ |
| α-methyl-Asparagine | —CH₃ | —CH₂C(═O)NH₂ |
| α-methyl-Aspartic | —CH₃ | —CH₂CO₂H |
| acid | | |
| α-methyl-Cysteine | —CH₃ | —CH₂SH |
| α-methyl-Glutamic | —CH₃ | —CH₂CH₂CO₂H |
| acid | | |
| α-methyl-Glutamine | —CH₃ | —CH₂CH₂C(═O)NH₂ |
| α-methyl-Histidine | —CH₃ | —CH₂-2-(1H-imidazole) |
| α-methyl-Isoleucine | —CH₃ | -sec-butyl |
| α-methyl-Leucine | —CH₃ | -iso-butyl |
| α-methyl-Lysine | —CH₃ | —CH₂CH₂CH₂CH₂NH₂ |
| α-methyl-Methionine | —CH₃ | —CH₂CH₂SCH₃ |
| α-methyl-Phenyl- | —CH₃ | —CH₂Ph |
| alanine | | |
| α-methyl-Proline | —CH₃ | -2-(pyrrolidine) |
| α-methyl-Serine | —CH₃ | —CH₂OH |
| α-methyl-Threonine | —CH₃ | —CH₂CH(OH)(CH₃) |
| α-methyl-Tryptophan | —CH₃ | —CH₂-3-(1H-indole) |
| α-methyl-Tyrosine | —CH₃ | —CH₂-(p-hydroxyphenyl) |
| α-methyl-Valine | —CH₃ | -isopropyl |
| Di-vinyl | —CH═CH₂ | —CH═CH₂ |
| Norleucine | —H | —CH₂CH₂CH₂CH₃ |

TABLE 3

| Exemplary unnatural | Amino acid side chains |
|---|---|
| alpha-amino acids | R and R′ is equal to hydrogen or —CH₃, and: |
| Terminally unsaturated | —(CH₂)_{g}—S—(CH₂)_{g}CH═CH_{2,} |
| alpha-amino acids and | —(CH₂)_{g}—O—(CH₂)_{g}CH═CH_{2,} |
| bis(alpha-amino acids) | —(CH₂)_{g}—NH—(CH₂)_{g}CH═CH_{2,} |
| (e.g., modified cysteine, | —(CH₂)_{g}—(C═O)—S—(CH₂)_{g}CH═CH_{2,} |
| modified lysine, | —(CH₂)_{g}—(C═O)—O—(CH₂)_{g}CH═CH_{2,} |
| modified tryptophan, | —(CH₂)_{g}—(C═O)—NH—(CH₂)_{g}CH═CH_{2,} |
| modified serine, | —CH₂CH₂CH₂CH₂—NH—(CH₂)_{g}CH═CH_{2,} |
| modified threonine, | —(C₆H₅)-p-O—(CH₂)_{g}CH═CH_{2,} |
| modified proline, | —CH(CH₃)—O—(CH₂)_{g}CH═CH_{2,} |
| modified histidine, | —CH₂CH(—O—CH═CH₂)(CH₃), |
| modified alanine, and | -histidine-N((CH₂)_{g}CH═CH₂), |
| the like). | -tryptophan-N((CH₂)_{g}CH═CH₂), and |
| | —(CH₂)_{g+1}(CH═CH₂), |
| | wherein each instance of g is, independently, |
| | 0 to 10. |

There are many known unnatural amino acids any of which may be included in the peptides of the present invention. See for example, S. Hunt, The Non-Protein Amino Acids: In Chemistry and Biochemistry of the Amino Acids, edited by G. C. Barrett, Chapman and Hall, 1985. Some non-limiting examples of unnatural amino acids include, but are not limited to, 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; CH₃), disubstituted phenylalanines, substituted tyrosines (e.g., further substituted with —C(═O)C₆H₅; —CF₃; —CN; -halo; —NO₂; CH₃), and statine. Additionally, the amino acids suitable for use in the present invention may be derivatized to include amino acid residues that are hydroxylated, phosphorylated, sulfonated, acylated, lapidated, farnesylated, and glycosylated, to name a few.

The term “amino acid side chain” refers to a group attached to the alpha- or beta-carbon of an amino acid, and includes, but is not limited to, any of the amino acid side chains as defined herein, and as provided in Tables 1 to 3. Exemplary amino acid side chains include, but are not limited to, methyl (as the alpha-amino acid side chain for alanine is methyl), 4-hydroxyphenylmethyl (as the alpha-amino acid side chain for tyrosine is 4-hydroxyphenylmethyl), and thiomethyl (as the alpha-amino acid side chain for cysteine is thiomethyl), etc.

A “terminally unsaturated amino acid side chain” refers to an amino acid side chain bearing a terminally unsaturated moiety, such as a substituted or unsubstituted, double bond (e.g., olefinic or alkenyl) or a triple bond (e.g., acetylenic or alkynyl), that can participate in a crosslinking reaction with another terminally unsaturated moiety in the polypeptide chain. In certain embodiments, a “terminally unsaturated amino acid side chain” is a terminal olefinic amino acid side chain. In certain embodiments, a “terminally unsaturated amino acid side chain” is a terminal acetylenic amino acid side chain. In certain embodiments, the terminal moiety of a “terminally unsaturated amino acid side chain” is not further substituted. Terminally unsaturated amino acid side chains include, but are not limited to, side chains as depicted in Table 3.

A “peptide” or “polypeptide” comprises a polymer of amino acid residues linked together by peptide (amide) bonds. The term(s), as used herein, refers to proteins, polypeptides, and peptide of any size, structure, or function. Typically, a peptide or polypeptide will be at least three amino acids long, e.g., at least 3 to 100 or more amino acids in length. A peptide or polypeptide may refer to an individual protein or a collection of proteins. Proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a polypeptide or protein may be modified, for example, by the addition of a chemical entity such as a carbohydrate group, a hydroxyl group, a lipid group, a phosphate group, a farnesyl group, an isofarnesyl group, a fatty acid group, a linker for conjugation, functionalization, or other modification. A polypeptide may also be a single molecule or may be a multi-molecular complex, such as a protein. A polypeptide or protein may be just a fragment of a naturally occurring protein or peptide. A polypeptide or protein may be naturally occurring, recombinant, or synthetic, or any combination thereof. As used herein “dipeptide” refers to two covalently linked amino acids.

The term “homologous” is a term that refers to polypeptides and proteins that are highly related at the level of the amino acid sequence. Polypeptides and proteins that are homologous to each other are termed homologues. Homologous may refer to the degree of sequence similarity between two sequences. Two polypeptide or protein sequences are considered to be homologous if least one stretch of at least 20 amino acids of the polypeptide or protein are at least about 50-60% identical, preferably about 70% identical. The homology percentage reflects the maximal homology possible between two sequences, i.e. the percent homology when the two sequences are so aligned as to have the greatest number of matched (homologous) positions. Homology can be readily calculated by known methods such as those described in: Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991. Methods commonly employed to determine homology between sequences include, but are not limited to those disclosed in Carillo, H., and Lipman, D., SIAM J Applied Math., 48:1073 (1988). Techniques for determining homology are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J Molec. Biol., 215, 403 (1990)).

As used herein, the term “salt” or “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts, e.g., cationic trisubstituted amino groups, e.g., as defined herein.

As used herein, when two entities are “conjugated” to one another they are linked by a direct or indirect covalent or non-covalent interaction. In certain embodiments, the association is covalent. In other embodiments, the association is non-covalent. Non-covalent interactions include, but are not limited to, hydrogen bonding, van der Waals interactions, hydrophobic interactions, magnetic interactions, and electrostatic interactions. An indirect covalent interaction is when two entities are covalently connected, optionally through a linker.

As used herein, a “label” refers to a moiety that has at least one element, isotope, or functional group incorporated into the moiety which enables detection of the inventive polypeptide to which the label is attached. Labels can be directly attached (ie, via a bond) or can be attached by a linker (e.g., such as, for example, a substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted arylene; substituted or unsubstituted heteroarylene; acylene, or any combination thereof, which can make up a linker). It will be appreciated that the label may be attached to the inventive polypeptide at any position that does not interfere with the biological activity or characteristic of the inventive polypeptide that is being detected.

In general, a label can fall into any one (or more) of five classes: a) a label which contains isotopic moieties, which may be radioactive or heavy isotopes, including, but not limited to, ²H, ³H, ¹³C, ¹⁴C, ¹⁵N, ¹⁸F, ³¹P, ³²P, ³⁵S, ⁶⁷Ga, ^{99m}Tc (Tc-99m), ¹¹¹In, ¹²³I, ¹²⁵I, ¹⁶⁹Yb, and ¹⁸⁶Re; b) a label which contains an immune moiety, which may be antibodies or antigens, which may be bound to enzymes (e.g., such as horseradish peroxidase); c) a label which is a colored, luminescent, phosphorescent, or fluorescent moieties (e.g., such as the fluorescent label FITC); d) a label which has one or more photoaffinity moieties; and e) a label which has a ligand moiety with one or more known binding partners (such as biotin-streptavidin, FK506-FKBP, etc.). Any of these type of labels as described above may also be referred to as “diagnostic agents” as defined herein.

In certain embodiments, such as in the identification of a biological target, label comprises a radioactive isotope, preferably an isotope which emits detectable particles, such as β particles. In certain embodiments, the label comprises one or more photoaffinity moieties for the direct elucidation of intermolecular interactions in biological systems. A variety of known photophores can be employed, most relying on photoconversion of diazo compounds, azides, or diazirines to nitrenes or carbenes (see, Bayley, H., Photogenerated Reagents in Biochemistry and Molecular Biology (1983), Elsevier, Amsterdam, the entire contents of which are incorporated herein by reference). In certain embodiments of the invention, the photoaffinity labels employed are o-, m- and p-azidobenzoyls, substituted with one or more halogen moieties, including, but not limited to 4-azido-2,3,5,6-tetrafluorobenzoic acid.

In certain embodiments, the label comprises one or more fluorescent moieties. In certain embodiments, the label is the fluorescent label FITC. In certain embodiments, the label comprises a ligand moiety with one or more known binding partners. In certain embodiments, the label comprises the ligand moiety biotin.

As used herein, a “diagnostic agent” refers to imaging agents. Exemplary imaging agents include, but are not limited to, those used in positron emissions tomography (PET), computer assisted tomography (CAT), single photon emission computerized tomography, x-ray, fluoroscopy, and magnetic resonance imaging (MRI); anti-emetics; and contrast agents. Exemplary diagnostic agents include but are not limited to, fluorescent moieties, luminescent moieties, magnetic moieties; gadolinium chelates (e.g., gadolinium chelates with DTPA, DTPA-BMA, DOTA and HP-DO3A), iron chelates, magnesium chelates, manganese chelates, copper chelates, chromium chelates, iodine-based materials useful for CAT and x-ray imaging, and radionuclides. Suitable radionuclides include, but are not limited to, ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸Y, ⁹⁷Ru, ¹⁰⁰Pd, ¹⁰¹mRh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁷Cu, ⁷⁵Br, ⁷⁷Br, ⁹⁹mTc, ¹⁴C, ¹³N, ¹⁵O, ³²P, ³³P, and ¹⁸F. Fluorescent and luminescent moieties include, but are not limited to, a variety of different organic or inorganic small molecules commonly referred to as “dyes,” “labels,” or “indicators.” Examples include, but are not limited to, fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin and derivatives thereof. Numerous fluorescent and luminescent dyes and proteins are known in the art (see, e.g., U.S. Patent Publication 2004/0067503; Valeur, B., “Molecular Fluorescence: Principles and Applications,” John Wiley and Sons, 2002; and Handbook of Fluorescent Probes and Research Products, Molecular Probes, 9^{th} edition, 2002).

As used herein “at least one instance” refers to at least 1, 2, 3, 4, or more instances.

### DETAILED DESCRIPTION OF CERTAIN EMBODIMENTS OF THE INVENTION

As generally described above, the present invention provides polypeptides comprising a stapled or stitched alpha-helical insulin receptor (IR) binding segment, and “unstapled” or “unstitched” precursor polypeptides thereof. Further provided are methods of making the stapled or stitched polypeptides, pharmaceutical compositions thereof, uses thereof, methods of using the stapled or stitched peptides, and methods of treating and/or preventing diabetes or pre-diabetes. In certain embodiments, the stabilized polypeptide binds to the ectodomain of the insulin receptor (IR). In certain embodiments, the stabilized polypeptide binds to site 1 of the IR. In certain embodiments, the stabilized polypeptide binds to the L1 domain of site 1 of the IR. In certain embodiments, the stabilized polypeptide binds to site 1 and site 2′ of the IR. In certain embodiments, the stabilized polypeptide binds to residues in the Fn0/Fn1 loop. The staples of the stabilized polypeptide are ideally situated in order to not interfere with binding of the polypeptide to the IR. In certain embodiments, the staples increase helicity of the stabilized polypeptide and enhance binding.

“Stapling” as used herein, is a process by which two terminally unsaturated amino acid side chains in a polypeptide chain react with each other in the presence of a ring closing metathesis (RCM) catalyst to generate a C—C double bonded cross-link between the two amino acids (a “staple”). See, e.g., Bernal et al., J. Am. Chem. Soc. (2007) 129: 2456-2457. In certain embodiments, the RCM catalyst is a ruthenuim catalyst. Suitable RCM catalysts are described in, for example, Grubbs et al., Acc. Chem. Res. 1995, 28, 446-452; U.S. Pat. No. 5,811,515; Schrock et al., Organometallics (1982) 1 1645; Gallivan et al., Tetrahedron Letters (2005) 46:2577-2580; Furstner et al., J. Am. Chem. Soc. (1999) 121:9453; and Chem. Eur. J. (2001) 7:5299. Stapling engenders constraint on a secondary structure, such as an alpha-helical structure. The length and geometry of the cross-link can be optimized to improve the yield of the desired secondary structure content. The constraint provided can, for example, prevent the secondary structure to unfold and/or can reinforce the shape of the secondary structure, and thus makes the secondary structure more stable. Stapled peptides may have increased half-lives in vivo and may have oral bioavailability.

A stapled polypeptide may contain more than one staple, i.e., two, three, four, five, six, seven, eight, nine, ten, or more staples. In certain embodiments, wherein the stapled polypeptide comprises more than one staple, the polypeptide may also be referred to as a “stitched” polypeptide. A stitched polypeptide is generated from a polypeptide comprising at least one central amino acid which comprises two terminally unsaturated amino acid side chains and at least two amino acids peripheral to (located on either side of) the central amino acid, each of which comprises at least one terminally unsaturated amino acid side chain. The “stitching” occurs when the central and peripheral amino acids react with each other in the presence of a ring closing metathesis catalyst to generate two C—C double bonded cross-links, i.e., one staple linking one peripheral amino acid to the central amino acid, and the other staple linking the other peripheral amino acid to the central amino acid, i.e., to provide a “stitch”. The concept of stapling and stitching is generally known in the art. See, e.g., U.S. Pat. Nos. 7,192,713; 7,723,469; 7,786,072; U.S. Patent Application Publication Nos: 2010-0184645; 2010-0168388; 2010-0081611; 2009-0176964; 2009-0149630; 2006-0008848; PCT Application Publication Nos: WO 2010/011313; WO 2008/121767; WO 2008/095063; WO 2008/061192; and WO 2005/044839, which depict stapling and stitching of polypeptides and are incorporated herein by reference.

In general, the precursor polypeptides to the stabilized polypeptides contemplated herein comprise an alpha-helical segment, wherein the precursor polypeptide and/or the stabilized polypeptide binds to the insulin receptor, and wherein the polypeptide comprises at least two amino acid moieties of Formula (i), and optionally, one amino acid of Formula (ii), as part of the polypeptide sequence:

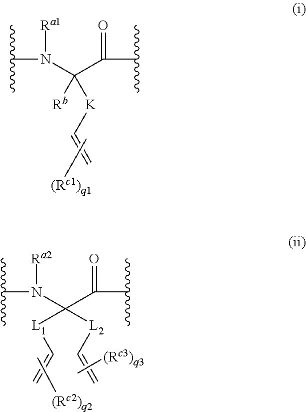

wherein:

each instance of K, L₁, and L₂, is, independently a bond or a group consisting of a combination of one or more of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted heterocyclene; substituted and unsubstituted carbocyclene; substituted and unsubstituted arylene; and substituted and unsubstituted heteroarylene;

each instance of R^{a1} and R^{a2} is, independently, hydrogen; substituted or unsubstituted aliphatic; substituted or unsubstituted heteroaliphatic; substituted or unsubstituted aryl; substituted or unsubstituted heteroaryl; acyl; or an amino protecting group;

R^{b} is hydrogen; substituted or unsubstituted aliphatic; substituted or unsubstituted heteroaliphatic; substituted or unsubstituted aryl; or substituted or unsubstituted heteroaryl;

each instance of R^{c1}, R^{c2}, and R^{c3} is independently hydrogen; substituted or unsubstituted aliphatic; substituted or unsubstituted heteroaliphatic; substituted or unsubstituted aryl; substituted or unsubstituted heteroaryl; acyl; substituted or unsubstituted hydroxyl; substituted or unsubstituted thiol; substituted or unsubstituted amino; azido; cyano; isocyano; halo; or nitro; and

each instance of q^{c1}, q^{c2}, and q^{c3} is independently 0, 1, or 2; or a pharmaceutically acceptable salt thereof.

In certain embodiments, the amino acids of Formula (i) and optionally Formula (ii) are amino acids of the alpha-helical segment.

Upon treatment of the precursor polypeptide with a RCM catalyst, a stabilized (stapled or stitched) polypeptide comprising an alpha-helical segment is generated, wherein the stabilized polypeptide binds to the insulin receptor, and wherein the polypeptide comprises at least two cross-linked (stapled) amino acids as shown in Formula (iii):

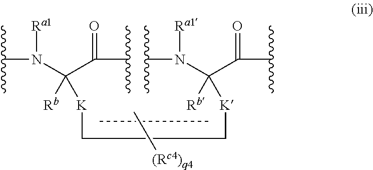

or at least three cross-linked (multiply stapled, stitched) amino acids as shown in Formula (iv):

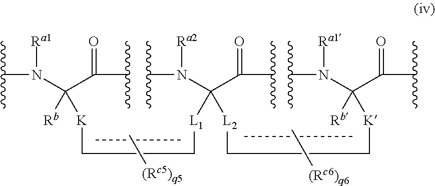

wherein:

each instance of K, K′, L₁, and L₂, is, independently a bond or a group consisting of a combination of one or more of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted heterocyclene; substituted and unsubstituted carbocyclene; substituted and unsubstituted arylene; and substituted and unsubstituted heteroarylene;

each instance of R^{a1}, R^{a1′}, and R^{a2} is, independently, hydrogen; substituted or unsubstituted aliphatic; substituted or unsubstituted heteroaliphatic; substituted or unsubstituted aryl; substituted or unsubstituted heteroaryl; acyl; or an amino protecting group;

each instance of R^{b} and R^{b′} is, independently, hydrogen; substituted or unsubstituted aliphatic; substituted or unsubstituted heteroaliphatic; substituted or unsubstituted aryl; substituted or unsubstituted heteroaryl;

each instance of [symbol] independently represents a single or double bond;

each instance of R^{c1}, R^{c2}, R^{c3}, R^{c4}, R^{c5}, and R^{c6} is independently hydrogen; substituted or unsubstituted aliphatic; substituted or unsubstituted heteroaliphatic; substituted or unsubstituted aryl; substituted or unsubstituted heteroaryl; acyl; substituted or unsubstituted hydroxyl; substituted or unsubstituted thiol; substituted or unsubstituted amino; azido; cyano; isocyano; halo; or nitro; and

each instance of q^{c1}, q^{c2}, q^{c3}, q^{c4}, q^{c5}, and q^{c6} is independently 0, 1, or 2; or a pharmaceutically acceptable salt thereof.

In certain embodiments, the two cross-linked amino acids of Formula (iii) or the three cross-linked amino acids of Formula (iv) are amino acids of the alpha-helical segment. Stabilization of the alpha-helical secondary structure by stapling or stitching results in, for example, increased alpha helicity, decreased susceptibility to enzymatic degradation, and/or increased thermal stability, as compared to the precursor polypeptide or the polypeptide without amino acids suitable for stapling or stitching.

In general, the polypeptide region targeting the IR is alpha-helical or substantially alpha-helical, and the staples or stitches stabilize this alpha-helical region. In certain embodiments, the polypeptides that target the IR comprise sequences that are approximately 5, 6, 7, 8, 9, 10, 11, 12, 13, 14, 15, 16, 17, 18, 19, 20, 21, 22, 23, 24, 25, 26, 27, 28, 29, or 30 amino acids long. In other embodiments, the polypeptides are approximately 5-10, 5-20, 5-30, 5-40, 5-50, 10-20, 10-16, 10-18, 12-18, 114-18, 15-17, 15-20, or 16-20 amino acids long. In certain embodiments, the polypeptides provided herein that target the IR comprise sequences that are derived from an alpha-helical segment of insulin. In certain embodiments, the polypeptides provided herein that target the IR comprise sequences that are derived from an alpha-helical segment of an insulin mimic, such as the polypeptide S371. The invention is based, in part, on the discovery that stapled versions of the alpha-helical IR modulator S371 have the ability to bind efficiently to the IR, thereby providing agents that can be used to modulate (agonize or antagonize) IR activity.

As used herein, the phrase “substantially alpha-helical” refers to a polypeptide adopting, on average, backbone (φ, ψ) dihedral angles in a range from about (−90°, −15°) to about (−35°, −70°). Alternatively, the phrase “substantially alpha-helical” refers to a polypeptide adopting dihedral angles such that the ψ dihedral angle of one residue and the (p dihedral angle of the next residue sums, on average, to about −80° to about −125°. In certain embodiments, the polypeptide adopts dihedral angles such that the ψ dihedral angle of one residue and the φ dihedral angle of the next residue sums, on average, to about −100° to about −110°. In certain embodiments, the polypeptide adopts dihedral angles such that the ψ dihedral angle of one residue and the φ dihedral angle of the next residue sums, on average, to about −105°. Furthermore, the phrase “substantially alpha-helical” may also refer to a polypeptide having at least 50%, at least 60%, at least 70%, at least 80%, at least 90%, or at least 95% of the amino acids provided in the polypeptide chain in an alpha-helical conformation, or with dihedral angles as specified herein. Confirmation of a polypeptide's alpha-helical secondary structure may be ascertained by known analytical techniques, such as x-ray crystallography, electron crystallography, fiber diffraction, fluorescence anisotropy, circular dichroism (CD), and nuclear magnetic resonance (NMR) spectroscopy.

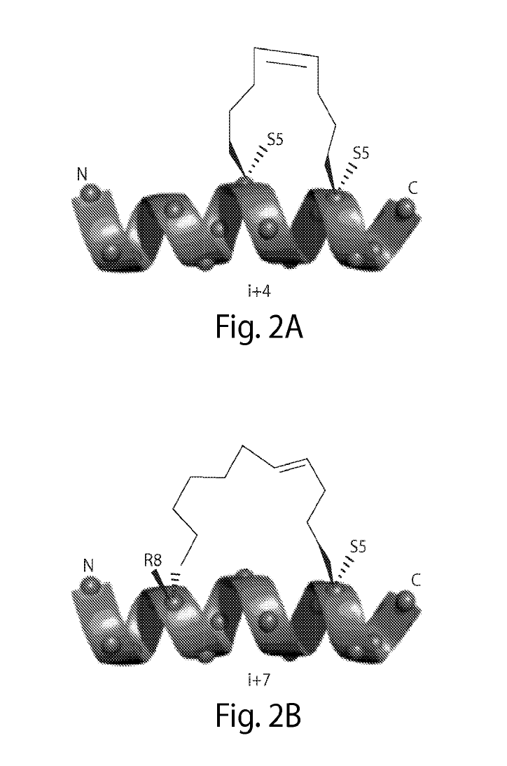

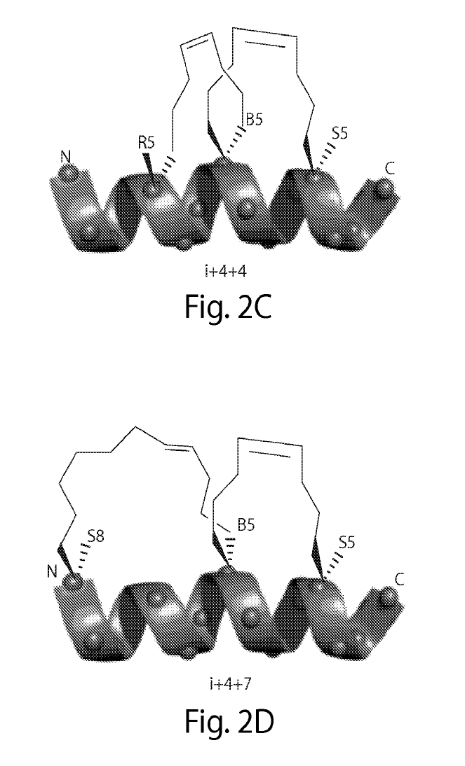

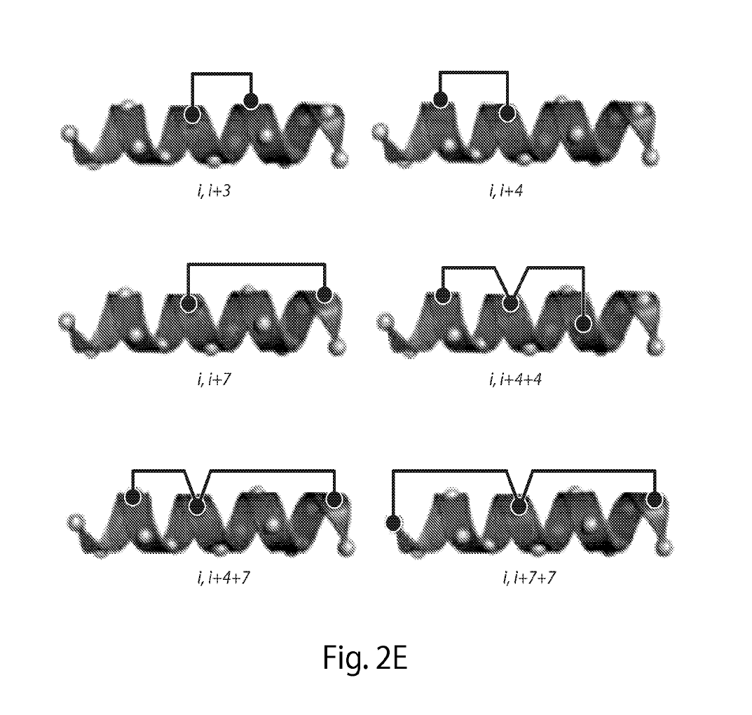

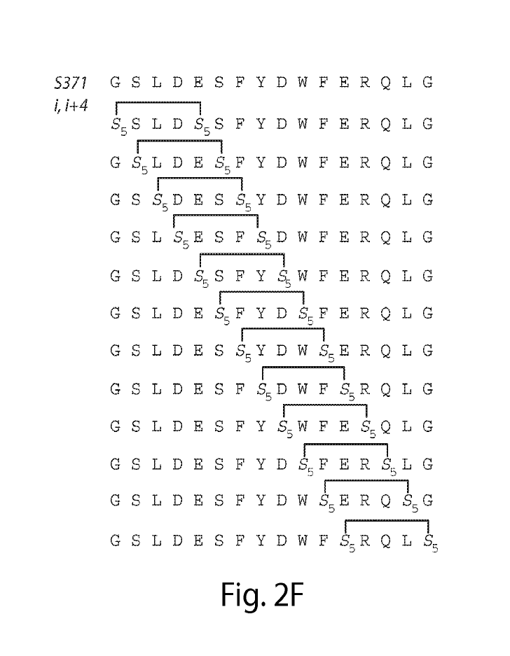

In general, the staple extends across the length of one or two helical turns (i.e., about 3, about 4, or about 7 amino acids), and amino acids positioned at i and i+3; i and i+4; or i and i+7 may be used for crosslinking. In certain embodiments, stapling may occur at the i,i+3 positions, i,i+4 positions, and/or i,i+7 positions. In certain embodiments, stitching may occur at the i,i+4+4 positions, the i,i+3+4 positions, the i,i+3+7 positions, or the i,i+4+7 positions. Examples of these stapling and stitching motifs is depicted in FIG. 2. In certain embodiments, the length of each staple (i.e., of a single staple or part of a stitch) is independently 6 to 20 atoms in length, i.e., 6, 7, 8, 9, 10, 11, 12, 13, 14, 15, 16, 17, 18, 19, or 20 atoms in length, measured from alpha carbon to alpha carbon and including each alpha carbon of each unnatural amino acid.

The staples of the polypeptide may further comprise additional synthetic modification(s). Any chemical or biological modification may be made. In certain embodiments, such modifications include reduction, oxidation, and nucleophilic or electrophilic additions to the double bond provided from a metathesis reaction of the cross-link to provide a synthetically modified stapled or stitched polypeptide. One of ordinary skill in the art will appreciate that a wide variety of conditions may be employed to promote such transformations, therefore, a wide variety of conditions are envisioned; see generally, March's Advanced Organic Chemistry: Reactions, Mechanisms, and Structure, M. B. Smith and J. March, 5^{th} Edition, John Wiley & Sons, 2001; Advanced Organic Chemistry, Part B: Reactions and Synthesis, Carey and Sundberg, 3^{rd} Edition, Plenum Press, New York, 1993; and Comprehensive Organic Transformations, R. C. Larock, 2^{nd} Edition, John Wiley & Sons, 1999, the entirety of each of which is hereby incorporated herein by reference. In other embodiments, the staple(s) of the polypeptide are not further modified.

Exemplary conditions may be any reagent reactive with a double bond. In certain embodiments, the reagent is able to react with a double bond, for example, via a hydrogenation, osmylation, hydroxylation (mono- or di-), amination, halogenation, cycloaddition (e.g., cyclopropanation, aziridination, epoxidation), oxy-mercuration, and/or a hydroboronation reaction, to provide a functionalized single bond. As one of ordinary skill in the art will clearly recognize, these above-described transformations will introduce functionalities compatible with the particular stabilized structures and the desired biological interactions; such functionalities include, but are not limited to, hydrogen; substituted or unsubstituted aliphatic; substituted or unsubstituted heteroaliphatic; substituted or unsubstituted aryl; substituted or unsubstituted heteroaryl; acyl; substituted or unsubstituted hydroxyl; substituted or unsubstituted amino; substituted or unsubstituted thiol, halo; cyano; nitro; azido; imino; oxo; and thiooxo.

Other modifications may further include conjugation of the stapled or stitched polypeptide, or a synthetically modified stapled or stitched polypeptide, with a biologically active agent, label, targeting moiety, diagnostic agent, anywhere on the polypeptide scaffold, e.g., such as at the N-terminus of the polypeptide, the C-terminus of the polypeptide, on an amino acid side chain of the polypeptide, or at one or more modified or unmodified stapled sites. Such modification may be useful in delivery of the peptide or biologically active agent to a cell, tissue, or organ. Such modifications may allow for targeting of the stabilized polypeptide to a particular type of cell or tissue. Conjugation of an agent (e.g., a label, a diagnostic agent, a biologically active agent, a targeting moiety) to the stapled polypeptide may be achieved in a variety of different ways. The agent may be covalently conjugated, directly or indirectly, to the polypeptide at the site of stapling, or to the N-terminus or the C-terminus of the polypeptide. Alternatively, the agent may be noncovalently conjugated, directly or indirectly, to the polypeptide at the site of stapling, or to the N-terminus or the C-terminus of the polypeptide, or any other site on the polypeptide. Indirect covalent conjugation is by means of one or more covalent bonds. Indirect non-covalent conjugation is by means of one or more non-covalent interactions. Conjugation may also be via a combination of non-covalent and covalent interactions. The agent may also be conjugated to the polypeptide through a linker. Any number of covalent bonds may be used in the conjugation of a biologically active agent and/or diagnostic agent to the inventive polypeptide of the present invention. Such bonds include amide linkages, ester linkages, disulfide linkages, carbon-carbon bonds, carbamate linkages, carbonate linkages, urea linkages, hydrazide linkages, and the like. In some embodiments, the bond is cleavable under physiological conditions (e.g., enzymatically cleavable, cleavable at a high or low pH, with heat, light, ultrasound, x-ray, etc.). However, in some embodiments, the bond is not cleavable.

Furthermore, the stapled or stitched polypeptide may be ligated, e.g., covalently conjugated, either directly or indirectly, to a protein, e.g., a recombinant protein, to provide a bifunctional polypeptide. See, e.g., PCT/US2009/004260. For example, one domain of the polypeptide, such as the alpha helix, acts as a targeting moiety that binds to the IR; the other domain is conjugated to a protein which is brought in close proximity to the IR.

### S371 Polypeptides and Precursors

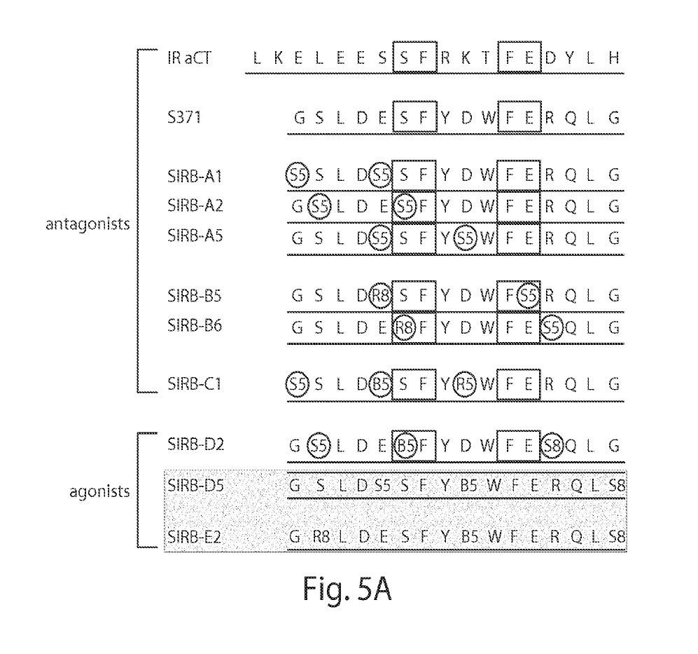

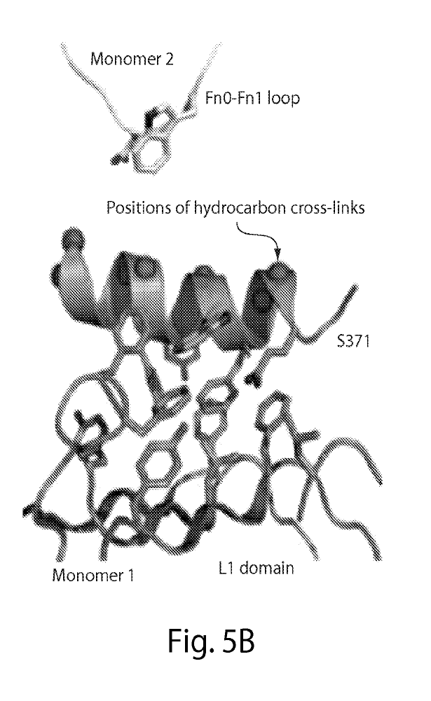

As a non-limiting example, the invention specifically contemplates stabilized forms of the polypeptide IR modulator S371, and unstitched and unstapled polypeptides thereof. The amino acid sequence of S371 is provided in FIG. 5. The sequence of the corresponding IR binding site is also provided in FIG. 5.

As generally described herein, provided is a precursor “unstapled” polypeptide of Formula (I):

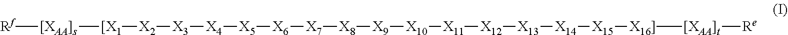

or a pharmaceutically acceptable salt thereof;
wherein:

each [X_{AA}] is independently a natural or unnatural amino acid;

s is 0 or an integer of between 1 to 50, inclusive;

t is 0 or an integer of between 1 to 50, inclusive;

R^{f} is an N-terminal group selected from the group consisting of hydrogen; substituted and unsubstituted aliphatic; substituted and unsubstituted heteroaliphatic; substituted and unsubstituted aryl; substituted and unsubstituted heteroaryl; acyl; a resin; an amino protecting group; and a label optionally joined by a linker, wherein the linker is a group consisting a combination of one or more of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted arylene; substituted and unsubstituted heteroarylene; and acylene;

R^{e} is a C-terminal group selected from the group consisting of hydrogen; substituted and unsubstituted aliphatic; substituted and unsubstituted heteroaliphatic; substituted and unsubstituted aryl; substituted and unsubstituted heteroaryl; —OR^{E}; —N(R^{E})₂; and —SR^{E}, wherein each instance of R^{E} is, independently, hydrogen; substituted or unsubstituted aliphatic; substituted or unsubstituted heteroaliphatic; substituted or unsubstituted aryl; substituted or unsubstituted heteroaryl; acyl; a resin; a protecting group; or two R^{E} groups taken together form an substituted or unsubstituted heterocyclic or substituted or unsubstituted heteroaryl ring;

X₁ is amino acid G or an amino acid of Formula (i);

X₂ is amino acid S or an amino acid of Formula (i);

X₃ is amino acid L;

X₄ is amino acid D;

X₅ is amino acid E, an amino acid of Formula (i), or an amino acid of Formula (ii);

X₆ is amino acid S, an amino acid of Formula (i), or an amino acid of Formula (ii);

X₇ is amino acid F;

X₈ is amino acid Y;

X₉ is amino acid D or an amino acid of Formula (i);

X₁₀ is amino acid W;

X₁₁ is amino acid F;

X₁₂ is amino acid E or an amino acid of Formula (i);

X₁₃ is amino acid R or an amino acid of Formula (i);

X₁₄ is amino acid Q;

X₁₅ is amino acid L; and

X₁₆ is amino acid G;

provided that the amino acid sequence comprises two independent occurrences of an amino acid of Formula (i), and/or one occurrence of Formula (ii) and two amino acids of Formula (i) peripheral thereto.

In certain embodiments, the amino acid sequence comprises two independent occurrences of an amino acid of Formula (i) separated by two (i,i+3) amino acids, three (i,i+4) amino acids, or six (i,i+7) amino acids, and/or one occurrence of Formula (ii) and two amino acids of Formula (i) peripheral thereto each separated by three (i,i+4+4) amino acids, separated by two and three amino acids (i,i+3+4), separated by two and six amino acids (i,i+3+7), or separated by three and six (i,i+4+7) amino acids.

Stapling of the polypeptide of Formula (I) by ring closing metathesis, and optionally synthetically modifying the resulting double bond of the staple, provides a stapled polypeptide of Formula (II):

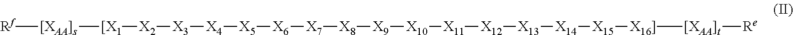

or a pharmaceutically acceptable salt thereof;
wherein:

each [X_{AA}] is independently a natural or unnatural amino acid;

s is 0 or an integer of between 1 and 50, inclusive;

t is 0 or an integer of between 1 and 50, inclusive;

R^{f} is an N-terminal group selected from the group consisting of hydrogen; substituted and unsubstituted aliphatic; substituted and unsubstituted heteroaliphatic; substituted and unsubstituted aryl; substituted and unsubstituted heteroaryl; acyl; a resin; an amino protecting group; and a label optionally joined by a linker, wherein the linker is a group consisting of one or more combinations of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted arylene; substituted and unsubstituted heteroarylene; and acylene;

R^{e} is a C-terminal group selected from the group consisting of hydrogen; substituted and unsubstituted aliphatic; substituted and unsubstituted heteroaliphatic; substituted and unsubstituted aryl; substituted and unsubstituted heteroaryl; —OR^{E}; —N(R^{E})₂; and —SR^{E}; wherein each instance of R^{E} is, independently, hydrogen; substituted or unsubstituted aliphatic; substituted or unsubstituted heteroaliphatic; substituted or unsubstituted aryl; substituted or unsubstituted heteroaryl; acyl; a resin; a protecting group; or two R^{E} groups taken together form an substituted or unsubstituted heterocyclic or substituted or unsubstituted heteroaryl ring;

X₁ is amino acid G or is an amino acid which forms together with another amino acid a staple of Formula (iii);

X₂ is amino acid S or is an amino acid which forms together with another amino acid a staple of Formula (iii);

X₃ is amino acid L;

X₄ is amino acid D;

X₅ is amino acid E, is an amino acid which forms together with another amino acid a staple of Formula (iii), or is an amino acid which forms together with two other amino acids a stitch of formula (iv);

X₆ is amino acid S, is an amino acid which forms together with another amino acid a staple of Formula (iii), or is an amino acid which forms together with two other amino acids a stitch of formula (iv);

X₇ is amino acid F;

X₈ is amino acid Y;

X₉ is amino acid D or is an amino acid which forms together with another amino acid a staple of Formula (iii);

X₁₀ is amino acid W;

X₁₁ is amino acid F;

X₁₂ is amino acid E or is an amino acid which forms together with another amino acid a staple of Formula (iii);

X₁₃ is amino acid R or is an amino acid which forms together with another amino acid a staple of Formula (iii);

X₁₄ is amino acid Q;

X₁₅ is amino acid L; and

X₁₆ is amino acid G;

provided that the amino acid sequence comprises at least one staple of Formula (iii); and/or at least one stitch of Formula (iv).

In certain embodiments, the amino acid sequence comprises at least one staple of Formula (iii) at the i,i+3 position, i,i+4 position, or the i,i+7 position; and/or at least one stitch of Formula (iv) at the i,i+4+4 position, the i,i+3+4 position, the i,i+3+7 position, or the i,i+4+7 position.

As generally understood herein, the amino acid region for each of Formula (I) and (II) [X₁-X₂-X₃-X₄-X₅-X₆-X₇-X₈-X₉-X₁₀-X₁₁-X₁₂-X₁₃-X₁₄-X₁₅-X₁₆] adopts an alpha-helical secondary structure, and stapling or stitching further stabilizes this structure.

Substitution of an amino acid for another amino acid sharing similar chemical properties is contemplated by the present invention. For example, methionine (M), alanine (A), leucine (L), glutamate (E), and lysine (K) have especially high alpha-helix forming propensities. In contrast, proline (P) and glycine (G) are alpha-helix disruptors, but proline (P) has also been found to be an initiator of alpha-helix formation. Arginine (R), histidine (H), and lysine (L) contain amino functionalized side chains which are basic and may be positively charged. Aspartic acid (D) and glutamic acid (E) contain carboxylic acid (—CO₂H) functionalized side chains which are acidic and may be negatively charged at physiological pH. Serine (S) and threonine (T) each contain hydroxyl (—OH) functionalized side chains. Asaparagine (N) and glutamine (G) each contain amide (—CONH₂) functionalized side chains. Alanine (A), valine (V), isoleucine (I), leucine (L), methionine (M), phenylalanine (F), and tryptophan (W) are classified as hydrophobic. Phenylalanine (F), tyrosine (Y), tryptophan (W), and histidine (H) include aromatic side chains.

The present invention contemplates one or more point mutations to the amino acid sequences, as recited above and herein, by substitution of one or more amino acids for one or more different amino acids. In certain embodiments, the polypeptide includes one, two, three, four, or five point mutations. In certain embodiments, the polypeptide includes one, two, three, four, five, or more additional amino acids. In certain embodiments, the polypeptide has one, two, three, four, or five amino acids removed from the sequence. In certain embodiments, the resulting amino acid sequence is at least 90%, at least 95%, at least 97%, at least 98%, or at least 99% homologous or identical to the amino acid sequence described herein; see, for example, the amino acids sequences depicted in FIG. 5. In certain embodiments, the polypeptides may be further modified to increase cell permeability, for example, by a) introducing an additional R, Q, or W residue, and/or b) adding one or more additional R, Q, or W residues at the N- and/or C-terminus of the polypeptide.

### Groups R^{f} and R^{e}

As generally defined above for Formula (I) and (II), R^{f} is an N-terminal group selected from the group consisting of hydrogen; substituted and unsubstituted aliphatic; substituted and unsubstituted heteroaliphatic; substituted and unsubstituted aryl; substituted and unsubstituted heteroaryl; acyl; a resin; an amino protecting group; and a label optionally joined to the polypeptide by a linker, wherein the linker is a group consisting of a combination of one or more of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted arylene; substituted and unsubstituted heteroarylene; and acylene.

In certain embodiments, R^{f} is hydrogen (e.g., to provide an —NH(R^{d}) terminal group). In certain embodiments, R^{f} is substituted or unsubstituted aliphatic (e.g., —CH₃, —CH₂CH₃). In certain embodiments, R^{f} is substituted or unsubstituted heteroaliphatic. In certain embodiments, R^{f} is substituted or unsubstituted aryl. In certain embodiments, R^{f} is substituted or unsubstituted heteroaryl. In certain embodiments, R^{f} is acyl (e.g., acetyl (—COCH₃)). In certain embodiments, R^{f} is a resin. In certain embodiments, R^{f} is an amino protecting group (e.g., -Boc, -Fmoc).

In certain embodiments, R^{f} comprises a label optionally joined by a linker to the polypeptide, wherein the linker is a group consisting of a combination of one or more of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted arylene; substituted and unsubstituted heteroarylene; and acylene.

Exemplary labels include, but are not limited to, FITC and biotin:

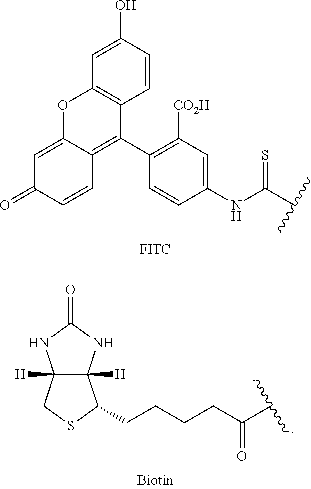

In certain embodiments, R^{f} is a label directly joined to the polypeptide (i.e., through a bond). In certain embodiments, R^{f} is a label indirectly joined to the polypeptide through a linker, wherein the linker is selected from the group consisting of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted arylene; substituted and unsubstituted heteroarylene; acylene; and combinations thereof.

In certain embodiments, the linker joining the label to the polypeptide is substituted or unsubstituted alkylene. In certain embodiments, the linker is substituted or unsubstituted alkenylene. In certain embodiments, the linker is substituted or unsubstituted alkynylene. In certain embodiments, the linker is substituted or unsubstituted heteroalkylene. In certain embodiments, the linker is substituted or unsubstituted heteroalkenylene. In certain embodiments, the linker is substituted or unsubstituted heteroalkynylene. In certain embodiments, the linker is substituted or unsubstituted arylene. In certain embodiments, the linker is substituted or unsubstituted heteroarylene. In certain embodiments, the linker is acylene.

As generally defined above for Formula (I) and (II), R^{e} is a C-terminal group selected from the group consisting of hydrogen; substituted and unsubstituted aliphatic; substituted and unsubstituted heteroaliphatic; substituted and unsubstituted aryl; substituted and unsubstituted heteroaryl; —OR^{E}, —N(R^{E})₂, and —SR^{E}, wherein each instance of R^{E} is, independently, hydrogen; substituted or unsubstituted aliphatic; substituted or unsubstituted heteroaliphatic; substituted or unsubstituted aryl; substituted or unsubstituted heteroaryl; acyl; a resin; a protecting group; or two R^{E} groups taken together form a substituted or unsubstituted heterocyclic or substituted or unsubstituted heteroaryl ring.

In certain embodiments, R^{e} is hydrogen, e.g., to provide an aldehyde (—CHO) as the C-terminal group. In certain embodiments, R^{e} is substituted or unsubstituted aliphatic; substituted or unsubstituted heteroaliphatic; substituted or unsubstituted aryl; or substituted or unsubstituted heteroaryl in order to provide a ketone as the C-terminal group.

In certain embodiments, R^{e} is —OR^{E}, and R^{E} is hydrogen; substituted or unsubstituted aliphatic; substituted or unsubstituted heteroaliphatic; substituted or unsubstituted aryl; substituted or unsubstituted heteroaryl; acyl; a resin; or a hydroxyl protecting group, e.g., to provide a carboxylic acid or ester C-terminal group. In certain embodiments, R^{e} is —OH. In certain embodiments, R^{e} is —OCH₃.

In certain embodiments, R^{e} is —SR^{E}, and R^{E} is hydrogen; substituted or unsubstituted aliphatic; substituted or unsubstituted heteroaliphatic; substituted or unsubstituted aryl; substituted or unsubstituted heteroaryl; acyl; a resin; or a suitable thiol protecting group, e.g., to provide a thioacid or thioester C-terminal group.

In certain embodiments, R^{e} is —N(R^{E})₂, and each instance of R^{E} is, independently, hydrogen; substituted or unsubstituted aliphatic; substituted or unsubstituted heteroaliphatic; substituted or unsubstituted aryl; substituted or unsubstituted heteroaryl; acyl; a resin; an amino protecting group; or two R^{E} groups together form a substituted or unsubstituted 5- to 6-membered heterocyclic or heteroaromatic ring, e.g., to provide an amide as the C-terminal group. In certain embodiments, R^{e} is —NH₂.

### Groups K, K′, L₁, and L₂

As generally defined above, each instance of K, K′, L₁, and L₂ is, independently, a bond or a group consisting of a combination of one or more of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted heterocyclene; substituted and unsubstituted carbocyclene; substituted and unsubstituted arylene; and substituted and unsubstituted heteroarylene.

As used herein, reference to a group consisting of “a combination” refers to a group comprising 1, 2, 3, 4 or more of the recited moieties. For example, the group may consist of an alkylene attached to a heteroalkylene, which may be further optionally attached to another alkylene. As used herein “at least one instance” refers to 1, 2, 3, 4, or more instances of the recited moiety.

In certain embodiments, K is a bond.

In certain embodiments, K is a group consisting of a combination of one or more of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted heterocyclene, substituted and unsubstituted carbocyclene, substituted and unsubstituted arylene; and substituted and unsubstituted heteroarylene.

In certain embodiments, K is a group which comprises at least one instance of substituted or unsubstituted alkylene, e.g., substituted or unsubstituted C₁₋₆alkylene, substituted or unsubstituted C₁₋₂alkylene, substituted or unsubstituted C₂₋₃alkylene, substituted or unsubstituted C₃₋₄alkylene, substituted or unsubstituted C₄₋₅alkylene, substituted or unsubstituted C₅₋₆alkylene, substituted or unsubstituted C₃₋₆alkylene, or substituted or unsubstituted C₄₋₆alkylene. Exemplary alkylene groups include unsubstituted alkylene groups such as methylene —CH₂—, ethylene —(CH₂)₂—, n-propylene —(CH₂)₃—, n-butylene —(CH₂)₄—, n-pentylene —(CH₂)₅—, and n-hexylene —(CH₂)₆—. In certain embodiments, K is substituted or unsubstituted alkylene.

In certain embodiments, K is a group which comprises at least one instance of substituted or unsubstituted alkenylene, e.g., substituted or unsubstituted C₂₋₆alkenylene, substituted or unsubstituted C₂₋₃alkenylene, substituted or unsubstituted C₃₋₄alkenylene, substituted or unsubstituted C₄₋₅alkenylene, or substituted or unsubstituted C₅₋₆alkenylene. In certain embodiments, K is substituted or unsubstituted alkenylene.

In certain embodiments, K is a group which comprises at least one instance of substituted or unsubstituted alkynylene, e.g., substituted or unsubstituted C₂₋₆alkynylene, substituted or unsubstituted C₂₋₃alkynylene, substituted or unsubstituted C₃₋₄alkynylene, substituted or unsubstituted C₄₋₅alkynylene, or substituted or unsubstituted C₅₋₆alkynylene. In certain embodiments, K is substituted or unsubstituted alkynylene.

In certain embodiments, K is a group which comprises at least one instance of substituted or unsubstituted heteroalkylene, e.g., substituted or unsubstituted heteroC₁₋₆alkylene, substituted or unsubstituted heteroC₁₋₂alkylene, substituted or unsubstituted heteroC₂₋₃alkylene, substituted or unsubstituted heteroC₃₋₄alkylene, substituted or unsubstituted heteroC₄₋₅alkylene, or substituted or unsubstituted heteroC₅₋₆alkylene. Exemplary heteroalkylene groups include unsubstituted alkylene groups such as —(CH₂)₂—O(CH₂)₂—, —OCH₂—, —O(CH₂)₂—, —O(CH₂)₃—, —O(CH₂)₄—, —O(CH₂)₅—, and —O(CH₂)₆—. In certain embodiments, K is substituted or unsubstituted heteroalkylene.

In certain embodiments, K is a group which comprises at least one instance of substituted or unsubstituted heteroalkenylene, e.g., substituted or unsubstituted heteroC₂₋₆alkenylene, substituted or unsubstituted heteroC₂₋₃alkenylene, substituted or unsubstituted heteroC₃₋₄alkenylene, substituted or unsubstituted heteroC₄₋₅alkenylene, or substituted or unsubstituted heteroC₅₋₆alkenylene. In certain embodiments, K is substituted or unsubstituted heteroalkenylene.

In certain embodiments, K is a group which comprises at least one instance of substituted or unsubstituted heteroalkynylene, e.g., substituted or unsubstituted heteroC₂₋₆alkynylene, substituted or unsubstituted heteroC₂₋₃alkynylene, substituted or unsubstituted heteroC₃₋₄alkynylene, substituted or unsubstituted heteroC₄₋₅alkynylene, or substituted or unsubstituted heteroC₅₋₆alkynylene. In certain embodiments, K is substituted or unsubstituted heteroalkynylene.

In certain embodiments, K is a group which comprises at least one instance of substituted or unsubstituted carbocyclylene, e.g., substituted or unsubstituted C₃₋₆carbocyclylene, substituted or unsubstituted C₃₋₄carbocyclylene, substituted or unsubstituted C₄₋₅ carbocyclylene, or substituted or unsubstituted C₅₋₆ carbocyclylene. In certain embodiments, K is substituted or unsubstituted carbocyclylene.

In certain embodiments, K is a group which comprises at least one instance of substituted or unsubstituted heterocyclylene, e.g., substituted or unsubstituted C₃₋₆ heterocyclylene, substituted or unsubstituted C₃₋₄ heterocyclylene, substituted or unsubstituted C₄₋₅ heterocyclylene, or substituted or unsubstituted C₅₋₆ heterocyclylene. In certain embodiments, K is substituted or unsubstituted heterocyclylene.

In certain embodiments, K is a group which comprises at least one instance of substituted or unsubstituted arylene, e.g., substituted or unsubstituted phenylene. In certain embodiments, K is substituted or unsubstituted arylene.

In certain embodiments, K is a group which comprises at least one instance of substituted or unsubstituted heteroarylene, e.g., substituted or unsubstituted 5- to 6-membered heteroarylene. In certain embodiments, K is substituted or unsubstituted heteroarylene.

In certain embodiments, K′ is a bond.

In certain embodiments, K′ is a group consisting of one or more combinations of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted heterocyclene; substituted and unsubstituted carbocyclene; substituted and unsubstituted arylene; substituted or unsubstituted heteroarylene.

In certain embodiments, K′ is a group which comprises at least one instance of substituted or unsubstituted alkylene, e.g., substituted or unsubstituted C₁₋₆alkylene, substituted or unsubstituted C₁₋₂alkylene, substituted or unsubstituted C₂₋₃alkylene, substituted or unsubstituted C₃₋₄alkylene, substituted or unsubstituted C₄₋₅alkylene, substituted or unsubstituted C₅₋₆alkylene, substituted or unsubstituted C₃₋₆alkylene, or substituted or unsubstituted C₄₋₆alkylene. Exemplary alkylene groups include unsubstituted alkylene groups such as methylene —CH₂—, ethylene —(CH₂)₂—, n-propylene —(CH₂)₃—, n-butylene —(CH₂)₄—, n-pentylene —(CH₂)₅—, and n-hexylene —(CH₂)₆—. In certain embodiments, K′ is substituted or unsubstituted alkylene.

In certain embodiments, K′ is a group which comprises at least one instance of substituted or unsubstituted alkenylene, e.g., substituted or unsubstituted C₂₋₆alkenylene, substituted or unsubstituted C₂₋₃alkenylene, substituted or unsubstituted C₃₋₄alkenylene, substituted or unsubstituted C₄₋₅alkenylene, or substituted or unsubstituted C₅₋₆alkenylene. In certain embodiments, K′ is substituted or unsubstituted alkenylene.

In certain embodiments, K′ is a group which comprises at least one instance of substituted or unsubstituted alkynylene, e.g., substituted or unsubstituted C₂₋₆alkynylene, substituted or unsubstituted C₂₋₃alkynylene, substituted or unsubstituted C₃₋₄alkynylene, substituted or unsubstituted C₄₋₅alkynylene, or substituted or unsubstituted C₅₋₆alkynylene. In certain embodiments, K′ is a substituted or unsubstituted alkynylene.

In certain embodiments, K′ is a group which comprises at least one instance of substituted or unsubstituted heteroalkylene, e.g., substituted or unsubstituted heteroC₁₋₆alkylene, substituted or unsubstituted heteroC₁₋₂alkylene, substituted or unsubstituted heteroC₂₋₃alkylene, substituted or unsubstituted heteroC₃₋₄alkylene, substituted or unsubstituted heteroC₄₋₅alkylene, or substituted or unsubstituted heteroC₅₋₆alkylene. Exemplary heteroalkylene groups include unsubstituted alkylene groups such as —(CH₂)₂—O(CH₂)₂—, —OCH₂—, —O(CH₂)₂—, —O(CH₂)₃—, —O(CH₂)₄—, —O(CH₂)₅—, and —O(CH₂)₆—. In certain embodiments, K′ is substituted or unsubstituted heteroalkylene.

In certain embodiments, K′ is a group which comprises at least one instance of substituted or unsubstituted heteroalkenylene, e.g., substituted or unsubstituted heteroC₂₋₆alkenylene, substituted or unsubstituted heteroC₂₋₃alkenylene, substituted or unsubstituted heteroC₃₋₄alkenylene, substituted or unsubstituted heteroC₄₋₅alkenylene, or substituted or unsubstituted heteroC₅₋₆alkenylene. In certain embodiments, K′ is substituted or unsubstituted heteroalkenylene.

In certain embodiments, K′ is a group which comprises at least one instance of substituted or unsubstituted heteroalkynylene, e.g., substituted or unsubstituted heteroC₂₋₆alkynylene, substituted or unsubstituted heteroC₂₋₃alkynylene, substituted or unsubstituted heteroC₃₋₄alkynylene, substituted or unsubstituted heteroC₄₋₅alkynylene, or substituted or unsubstituted heteroC₅₋₆alkynylene. In certain embodiments, K′ is substituted or unsubstituted heteroalkynylene.

In certain embodiments, K′ is a group which comprises at least one instance of substituted or unsubstituted carbocyclylene, e.g., substituted or unsubstituted C₃₋₆carbocyclylene, substituted or unsubstituted C₃₋₄carbocyclylene, substituted or unsubstituted C₄₋₅ carbocyclylene, or substituted or unsubstituted C₅₋₆ carbocyclylene. In certain embodiments, K′ is substituted or unsubstituted carbocyclylene.

In certain embodiments, K′ is a group which comprises at least one instance of substituted or unsubstituted heterocyclylene, e.g., substituted or unsubstituted C₃₋₆ heterocyclylene, substituted or unsubstituted C₃₋₄ heterocyclylene, substituted or unsubstituted C₄₋₅ heterocyclylene, or substituted or unsubstituted C₅₋₆ heterocyclylene. In certain embodiments, K′ is substituted or unsubstituted heterocyclylene.

In certain embodiments, K′ is a group which comprises at least one instance of substituted or unsubstituted arylene, e.g., substituted or unsubstituted phenylene. In certain embodiments, K′ is substituted or unsubstituted arylene.

In certain embodiments, K′ is a group which comprises at least one instance of substituted or unsubstituted heteroarylene, e.g., substituted or unsubstituted 5- to 6-membered heteroarylene. In certain embodiments, K′ is substituted or unsubstituted heteroarylene.

In certain embodiments, each instance of K and K′ is the same. In certain embodiments, each instance of K and K′ is different.

In certain embodiments, L₁ is a bond.

In certain embodiments, L₁ is a group consisting of one or more combinations of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted heterocyclene; substituted and unsubstituted carbocyclene; substituted and unsubstituted arylene; and substituted or unsubstituted heteroarylene.

In certain embodiments, L₁ is a group which comprises at least one instance of substituted or unsubstituted alkylene, e.g., substituted or unsubstituted C₁₋₆alkylene, substituted or unsubstituted C₁₋₂alkylene, substituted or unsubstituted C₂₋₃alkylene, substituted or unsubstituted C₃₋₄alkylene, substituted or unsubstituted C₄₋₅alkylene, substituted or unsubstituted C₅₋₆alkylene, substituted or unsubstituted C₃₋₆alkylene, or substituted or unsubstituted C₄₋₆alkylene. Exemplary alkylene groups include, but are not limited to, unsubstituted alkylene groups such as methylene —CH₂—, ethylene —(CH₂)₂—, n-propylene —(CH₂)₃—, n-butylene —(CH₂)₄—, n-pentylene —(CH₂)₅—, and n-hexylene —(CH₂)₆—. In certain embodiments, L₁ is substituted or unsubstituted alkylene.

In certain embodiments, L₁ is a group which comprises at least one instance of substituted or unsubstituted alkenylene, e.g., substituted or unsubstituted C₂₋₆alkenylene, substituted or unsubstituted C₂₋₃alkenylene, substituted or unsubstituted C₃₋₄alkenylene, substituted or unsubstituted C₄₋₅alkenylene, or substituted or unsubstituted C₅₋₆alkenylene. In certain embodiments, L₁ is substituted or unsubstituted alkenylene.

In certain embodiments, L₁ is a group which comprises at least one instance of substituted or unsubstituted alkynylene, e.g., substituted or unsubstituted C₂₋₆alkynylene, substituted or unsubstituted C₂₋₃alkynylene, substituted or unsubstituted C₃₋₄alkynylene, substituted or unsubstituted C₄₋₅alkynylene, or substituted or unsubstituted C₅₋₆alkynylene. In certain embodiments, L₁ is substituted or unsubstituted alkynylene.

In certain embodiments, L₁ is a group which comprises at least one instance of substituted or unsubstituted heteroalkylene, e.g., substituted or unsubstituted heteroC₁₋₆alkylene, substituted or unsubstituted heteroC₁₋₂alkylene, substituted or unsubstituted heteroC₂₋₃alkylene, substituted or unsubstituted heteroC₃₋₄alkylene, substituted or unsubstituted heteroC₄₋₅alkylene, or substituted or unsubstituted heteroC₅₋₆alkylene. Exemplary heteroalkylene groups include, but are not limited to, unsubstituted alkylene groups such as —(CH₂)₂—O(CH₂)₂—, —OCH₂—, —O(CH₂)₂—, —O(CH₂)₃—, —O(CH₂)₄—, —O(CH₂)₅—, and —O(CH₂)₆—. In certain embodiments, L₁ is substituted or unsubstituted heteroalkylene.

In certain embodiments, L₁ is a group which comprises at least one instance of substituted or unsubstituted heteroalkenylene, e.g., substituted or unsubstituted heteroC₂₋₆alkenylene, substituted or unsubstituted heteroC₂₋₃alkenylene, substituted or unsubstituted heteroC₃₋₄alkenylene, substituted or unsubstituted heteroC₄₋₅alkenylene, or substituted or unsubstituted heteroC₅₋₆alkenylene. In certain embodiments, L₁ is substituted or unsubstituted heteroalkenylene.

In certain embodiments, L₁ is a group which comprises at least one instance of substituted or unsubstituted heteroalkynylene, e.g., substituted or unsubstituted heteroC₂₋₆alkynylene, substituted or unsubstituted heteroC₂₋₃alkynylene, substituted or unsubstituted heteroC₃₋₄alkynylene, substituted or unsubstituted heteroC₄₋₅alkynylene, or substituted or unsubstituted heteroC₅₋₆alkynylene. In certain embodiments, L₁ is substituted or unsubstituted heteroalkynylene.

In certain embodiments, L₁ is a group which comprises at least one instance of substituted or unsubstituted carbocyclylene, e.g., substituted or unsubstituted C₃₋₆carbocyclylene, substituted or unsubstituted C₃₋₄carbocyclylene, substituted or unsubstituted C₄₋₅ carbocyclylene, or substituted or unsubstituted C₅₋₆ carbocyclylene. In certain embodiments, L₁ is substituted or unsubstituted carbocyclylene.

In certain embodiments, L₁ is a group which comprises at least one instance of substituted or unsubstituted heterocyclylene, e.g., substituted or unsubstituted C₃₋₆ heterocyclylene, substituted or unsubstituted C₃₋₄ heterocyclylene, substituted or unsubstituted C₄₋₅ heterocyclylene, or substituted or unsubstituted C₅₋₆ heterocyclylene. In certain embodiments, L₁ is substituted or unsubstituted heterocyclylene.

In certain embodiments, L₁ is a group which comprises at least one instance of substituted or unsubstituted arylene, e.g., substituted or unsubstituted phenylene. In certain embodiments, L₁ is substituted or unsubstituted arylene.

In certain embodiments, L₁ is a group which comprises at least one instance of substituted or unsubstituted heteroarylene, e.g., substituted or unsubstituted 5- to 6-membered heteroarylene. In certain embodiments, L₁ is substituted or unsubstituted heteroarylene.

In certain embodiments, L₂ is a bond.

In certain embodiments, L₂ is a group consisting of one or more combinations of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted heterocyclene; substituted and unsubstituted carbocyclene; substituted and unsubstituted arylene; and substituted or unsubstituted heteroarylene.

In certain embodiments, L₂ is a group which comprises at least one instance of substituted or unsubstituted alkylene, e.g., substituted or unsubstituted C₁₋₆alkylene, substituted or unsubstituted C₁₋₂alkylene, substituted or unsubstituted C₂₋₃alkylene, substituted or unsubstituted C₃₋₄alkylene, substituted or unsubstituted C₄₋₅alkylene, substituted or unsubstituted C₅₋₆alkylene, substituted or unsubstituted C₃₋₆alkylene, or substituted or unsubstituted C₄₋₆alkylene. Exemplary alkylene groups include, but are not limited to, unsubstituted alkylene groups such as methylene —CH₂—, ethylene —(CH₂)₂—, n-propylene —(CH₂)₃—, n-butylene —(CH₂)₄—, n-pentylene —(CH₂)₅—, and n-hexylene —(CH₂)₆—. In certain embodiments, L₂ is substituted or unsubstituted alkylene.

In certain embodiments, L₂ is a group which comprises at least one instance of substituted or unsubstituted alkenylene, e.g., substituted or unsubstituted C₂₋₆alkenylene, substituted or unsubstituted C₂₋₃alkenylene, substituted or unsubstituted C₃₋₄alkenylene, substituted or unsubstituted C₄₋₅alkenylene, or substituted or unsubstituted C₅₋₆alkenylene. In certain embodiments, L₂ is substituted or unsubstituted alkenylene.

In certain embodiments, L₂ is a group which comprises at least one instance of substituted or unsubstituted alkynylene, e.g., substituted or unsubstituted C₂₋₆alkynylene, substituted or unsubstituted C₂₋₃alkynylene, substituted or unsubstituted C₃₋₄alkynylene, substituted or unsubstituted C₄₋₅alkynylene, or substituted or unsubstituted C₅₋₆alkynylene. In certain embodiments, L₂ is substituted or unsubstituted alkynylene.

In certain embodiments, L₂ is a group which comprises at least one instance of substituted or unsubstituted heteroalkylene, e.g., substituted or unsubstituted heteroC₁₋₆alkylene, substituted or unsubstituted heteroC₁₋₂alkylene, substituted or unsubstituted heteroC₂₋₃alkylene, substituted or unsubstituted heteroC₃₋₄alkylene, substituted or unsubstituted heteroC₄₋₅alkylene, or substituted or unsubstituted heteroC₅₋₆alkylene. Exemplary heteroalkylene groups include, but are not limited to, unsubstituted alkylene groups such as —(CH₂)₂—O(CH₂)₂—, —OCH₂—, —O(CH₂)₂—, —O(CH₂)₃—, —O(CH₂)₄—, —O(CH₂)₅—, and —O(CH₂)₆—. In certain embodiments, L₂ is substituted or unsubstituted heteroalkylene.

In certain embodiments, L₂ is a group which comprises at least one instance of substituted or unsubstituted heteroalkenylene, e.g., substituted or unsubstituted heteroC₂₋₆alkenylene, substituted or unsubstituted heteroC₂₋₃alkenylene, substituted or unsubstituted heteroC₃₋₄alkenylene, substituted or unsubstituted heteroC₄₋₅alkenylene, or substituted or unsubstituted heteroC₅₋₆alkenylene. In certain embodiments, L₂ is substituted or unsubstituted heteroalkenylene.

In certain embodiments, L₂ is a group which comprises at least one instance of substituted or unsubstituted heteroalkynylene, e.g., substituted or unsubstituted heteroC₂₋₆alkynylene, substituted or unsubstituted heteroC₂₋₃ alkynylene, substituted or unsubstituted heteroC₃₋₄alkynylene, substituted or unsubstituted heteroC₄₋₅alkynylene, or substituted or unsubstituted heteroC₅₋₆alkynylene. In certain embodiments, L₂ is substituted or unsubstituted heteroalkynylene.

In certain embodiments, L₂ is a group which comprises at least one instance of substituted or unsubstituted carbocyclylene, e.g., substituted or unsubstituted C₃₋₆carbocyclylene, substituted or unsubstituted C₃₋₄carbocyclylene, substituted or unsubstituted C₄₋₅ carbocyclylene, or substituted or unsubstituted C₅₋₆ carbocyclylene. In certain embodiments, L₂ is substituted or unsubstituted carbocyclylene.

In certain embodiments, L₂ is a group which comprises at least one instance of substituted or unsubstituted heterocyclylene, e.g., substituted or unsubstituted C₃₋₆ heterocyclylene, substituted or unsubstituted C₃₋₄ heterocyclylene, substituted or unsubstituted C₄₋₅ heterocyclylene, or substituted or unsubstituted C₅₋₆ heterocyclylene. In certain embodiments, L₂ is substituted or unsubstituted heterocyclylene.

In certain embodiments, L₂ is a group which comprises at least one instance of substituted or unsubstituted arylene, e.g., substituted or unsubstituted phenylene. In certain embodiments, L₂ is substituted or unsubstituted arylene.

In certain embodiments, L₂ is a group which comprises at least one instance of substituted or unsubstituted heteroarylene, e.g., substituted or unsubstituted 5- to 6-membered heteroarylene. In certain embodiments, L₂ is substituted or unsubstituted heteroarylene.

In certain embodiments, each instance of L₁ and L₂ is the same. In certain embodiments, each instance of L₁ and L₂ is different.

In certain embodiments, each instance of L₁ and K are the same. In certain embodiments, each instance of L₁ and K are different.

In certain embodiments, each instance of L₂ and K′ are the same. In certain embodiments, each instance of L₂ and K′ are different.

### Group R^{a}

As generally defined above, each instance of R^{a1}, R^{a1′}, and R^{a2} is, independently, hydrogen; substituted or unsubstituted aliphatic; substituted or unsubstituted heteroaliphatic; substituted or unsubstituted aryl; substituted or unsubstituted heteroaryl; acyl; or an amino protecting group.

In certain embodiments, R^{a1} is hydrogen.

In certain embodiments, R^{a1} is substituted or unsubstituted aliphatic; i.e., substituted or unsubstituted alkyl, alkenyl, alkynyl, or carbocyclyl.

In certain embodiments, R^{a1} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{a1} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). In certain embodiments, R^{a1} is —CH₃.

In certain embodiments, R^{a1} is substituted or unsubstituted heteroaliphatic; i.e., substituted or unsubstituted heteroalkyl, heteroalkenyl, heteroalkynyl, or heterocyclyl.

In certain embodiments, R^{a1} is substituted or unsubstituted aryl.

In certain embodiments, R^{a1} is substituted or unsubstituted heteroaryl.

In certain embodiments, R^{a1} is acyl, e.g., acetyl (—C(═O)CH₃).

In certain embodiments, R^{a1} is an amino protecting group.

In certain embodiments, R^{a1′} is hydrogen.

In certain embodiments, R^{a1′} is substituted or unsubstituted aliphatic; i.e., substituted or unsubstituted alkyl, alkenyl, alkynyl, or carbocyclyl.

In certain embodiments, R^{a1′} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{a1′} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). In certain embodiments, R^{a1′} is —CH₃.

In certain embodiments, R^{a1′} is substituted or unsubstituted heteroaliphatic; i.e., substituted or unsubstituted heteroalkyl, heteroalkenyl, heteroalkynyl, or heterocyclyl.

In certain embodiments, R^{a1′} is substituted or unsubstituted aryl.

In certain embodiments, R^{a1′} is substituted or unsubstituted heteroaryl.

In certain embodiments, R^{a1′} is acyl, e.g., acetyl (—C(═O)CH₃).

In certain embodiments, R^{a1′} is an amino protecting group.

In certain embodiments, R^{a2} is hydrogen.

In certain embodiments, R^{a2} is substituted or unsubstituted aliphatic; i.e., substituted or unsubstituted alkyl, alkenyl, alkynyl, or carbocyclyl.

In certain embodiments, R^{a2} is substituted or unsubstituted alkyl, e.g., substituted or unsubstituted C₁₋₆ alkyl, substituted or unsubstituted C₁₋₂alkyl, substituted or unsubstituted C₂₋₃ alkyl, substituted or unsubstituted C₃₋₄alkyl, substituted or unsubstituted C₄₋₅alkyl, or substituted or unsubstituted C₅₋₆alkyl. Exemplary R^{a2} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). In certain embodiments, R^{a2} is —CH₃.

In certain embodiments, R^{a2} is substituted or unsubstituted heteroaliphatic; i.e., substituted or unsubstituted heteroalkyl, heteroalkenyl, heteroalkynyl, or heterocyclyl.

In certain embodiments, R^{a2} is substituted or unsubstituted aryl.

In certain embodiments, R^{a2} is substituted or unsubstituted heteroaryl.

In certain embodiments, R^{a2} is acyl, e.g., acetyl (—C(═O)CH₃).

In certain embodiments, R^{a2} is an amino protecting group.

In certain embodiments, each instance of R^{a1} and R^{a1′} is, independently, hydrogen, C₁₋₆alkyl (e.g., methyl), or acyl. In certain embodiments, each instance of R^{a1} and R^{a1′} is hydrogen.

In certain embodiments, each instance of R^{a1}, R^{a1′}, and R^{a2} is, independently, hydrogen, C₁₋₆alkyl (e.g., methyl), or acyl. In certain embodiments, each instance of R^{a1}, R^{a1′}, and R^{a2} is, independently, hydrogen, methyl, or acetyl. In certain embodiments, each instance of R^{a1}, R^{a1′}, and R^{a2} is, independently, hydrogen or methyl. In certain embodiments, each instance of R^{a1}, R^{a1′}, and R^{a2} is hydrogen. In certain embodiments, each instance of R^{a1}, R^{a1′}, and R^{a2} is methyl.

### Group R^{b}

As generally defined above, each instance of R^{b} and R^{b′} is, independently, hydrogen; substituted or unsubstituted aliphatic; substituted or unsubstituted heteroaliphatic; substituted or unsubstituted aryl; substituted or unsubstituted heteroaryl.

In certain embodiments, R^{b} is hydrogen.

In certain embodiments, R^{b} is substituted or unsubstituted aliphatic; i.e., substituted or unsubstituted alkyl, alkenyl, alkynyl, or carbocyclyl.

In certain embodiments, R^{b} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{b} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). In certain embodiments, R^{b} is —CH₃.

In certain embodiments, R^{b} is substituted or unsubstituted heteroaliphatic, i.e., substituted or unsubstituted heteroalkyl, heteroalkenyl, heteroalkynyl, or heterocyclyl.

In certain embodiments, R^{b} is substituted or unsubstituted aryl.

In certain embodiments, R^{b} is substituted or unsubstituted heteroaryl.

In certain embodiments, R^{b′} is hydrogen.

In certain embodiments, R^{b′} is substituted or unsubstituted aliphatic; i.e., substituted or unsubstituted alkyl, alkenyl, alkynyl, or carbocyclyl.

In certain embodiments, R^{b′} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{b′} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). In certain embodiments, R^{b′} is —CH₃.

In certain embodiments, R^{b′} is substituted or unsubstituted heteroaliphatic, i.e., substituted or unsubstituted heteroalkyl, heteroalkenyl, heteroalkynyl, or heterocyclyl.

In certain embodiments, R^{b′} is substituted or unsubstituted aryl.

In certain embodiments, R^{b′} is substituted or unsubstituted heteroaryl.

In certain embodiments, each instance of R^{b} and R^{b′} is, independently, hydrogen or substituted or unsubstituted aliphatic. In certain embodiments, each instance of R^{b} and R^{b′} is, independently, hydrogen or C₁₋₆alkyl. In certain embodiments, each instance of R^{b} and R^{b′} is, independently, hydrogen or —CH₃. In certain embodiments, each instance of R^{b} and R^{b′} is hydrogen. In certain embodiments, each instance of R^{b} and R^{b′} is —CH₃.

### Groups , R^{c} and q

As generally defined above, each instance of [symbol] independently represents a single or double bond; each instance of R^{c1}, R^{c2}, R^{c3}, R^{c4}, R^{c5}, and R^{c6} is independently hydrogen; substituted or unsubstituted aliphatic; substituted or unsubstituted heteroaliphatic; substituted or unsubstituted aryl; substituted or unsubstituted heteroaryl; acyl; substituted or unsubstituted hydroxyl; substituted or unsubstituted thiol; substituted or unsubstituted amino; azido; cyano; isocyano; halo; or nitro; and each instance of q^{c1}, q^{c2}, q^{c3}, q^{c4}, q^{c5}, and q^{c6} is independently 0, 1, or 2.

In certain embodiments, at least one instance of [symbol] is a single bond. In certain embodiments, each instance of [symbol] represents a single bond.

In certain embodiments, at least one instance of [symbol] is a double bond. In certain embodiments, each instance of [symbol] represents a double bond.

In certain embodiments, each instance of q^{c1}, q^{c2}, and q^{c3} is 0, and thus each instance of R^{c1}, R^{c2}, and R^{c3} is absent to provide an unsubstituted terminally unsaturated moiety. In certain embodiments at least one instance of q^{c1}, q^{c2}, and q^{c3} is 1, and thus at least one instance of R^{c1}, R^{c2}, and R^{c3} is a non-hydrogen substituent.

In certain embodiments, and each instance of q^{c4}, q^{c5}, and q^{c6} is 0, and thus each instance of R^{c4}, R^{c5}, and R^{c6} is absent to provide an unsubstituted crosslink. In certain embodiments at least one instance of q^{c4}, q^{c5}, and q^{c6} is 1, and thus at least one instance of R^{c4}, R^{c5}, and R^{c6} is a non-hydrogen substituent.

### [X_{AA}], s, and t

As generally defined above for Formula (I) and (II), each instance of X_{AA} is independently a natural amino acid or an unnatural amino acid, i.e., of the formula:

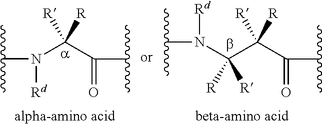

wherein each instance of R and R′ independently are selected from the group consisting of hydrogen; substituted and unsubstituted aliphatic, substituted and unsubstituted heteroaliphatic, substituted and unsubstituted aryl, substituted and unsubstituted heteroaryl; and R^{a} is hydrogen; substituted or unsubstituted aliphatic; substituted or unsubstituted heteroaliphatic; substituted or unsubstituted aryl; substituted or unsubstituted heteroaryl; acyl; or an amino protecting group.

In certain embodiments, R^{a} is an amino protecting group. In certain embodiments, R^{a} is hydrogen. In certain embodiments, R^{a} is —CH₃. In certain embodiments, R^{a} is acetyl.

In certain embodiments, R and R′ are groups as listed in Tables 1 or 2. For example, in certain embodiments, each instance of X_{AA} is independently a natural amino acid (e.g., selected from a natural alpha-amino acid as listed in Table 1 or a natural beta-amino acid, e.g., beta-alanine) or an unnatural amino acid (e.g., selected from an unnatural alpha-amino acid as listed in Table 2). In certain embodiments, each instance of X_{AA} is independently a natural alpha-amino acid or natural beta-amino acid. In certain embodiments, each instance of X_{AA} is independently a natural alpha amino acid as listed in Table 1. In certain embodiments, each instance of X_{AA} is a natural alpha amino acid independently selected from the group consisting of K, E, F, R, D, T, P, A, Y, H, and Q. However, in certain embodiments, at least one instance of X_{AA} is an unnatural amino acid, e.g., an unnatural alpha-amino acid as listed in Table 2.

As generally defined above for Formula (I) and (II), s and t define the number of amino acids X_{AA} at the N-terminus and C-terminus, respectively. In certain embodiments, s is 0 or an integer between 1 and 50, inclusive; between 1 and 40, inclusive; between 1 and 30, inclusive; between 1 and 20, inclusive; between 1 and 10, inclusive; or between 1 and 5 (e.g., 1, 2, 3, 4, or 5), inclusive. In certain embodiments, s is 0, 1, 2, 3, or 4. In certain embodiments, t is 0 or an integer between 1 and 50, inclusive; between 1 and 40, inclusive; between 1 and 30, inclusive; between 1 and 20, inclusive; between 1 and 10, inclusive; or between 1 and 5 (e.g., 1, 2, 3, 4, or 5), inclusive. In certain embodiments, t is 0, 1, 2, 3, or 4.

In certain embodiments, s and t are both 0.

Formulae (i), (ii), (iii), and (iv)

As generally defined above, the polypeptides contemplated herein comprise at least two amino acid moieties of Formula (i), and optionally, one amino acid of Formula (ii), as part of the polypeptide sequence:

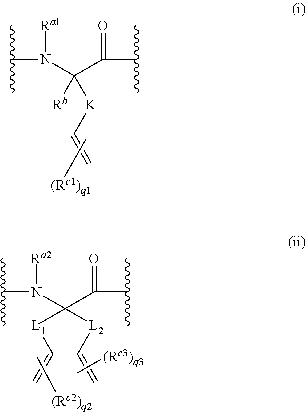

which upon treatment of the precursor polypeptide with a RCM catalyst, provides a polypeptide comprising a staple of Formula (iii):

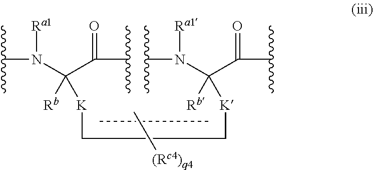

or a polypeptide comprising multiple staples (a “stitch”) of Formula (iv):

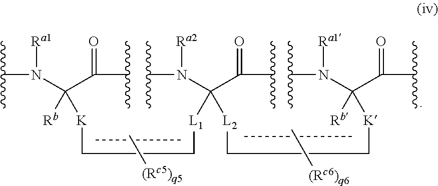

In certain embodiments of Formula (i) and (ii), wherein q1 and q2 are 0, provided are amino acids of Formula (i-a) and (ii-a):

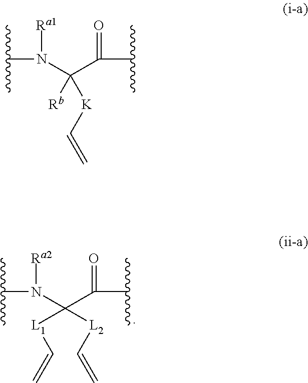

In certain embodiments of Formula (i-a), wherein R^{b} is methyl, provided is an amino acid of Formula (i-b):

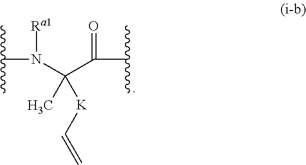

In certain embodiments of Formula (i-b), wherein K is C₁₋₆alkylene, provided is an amino acid of Formula (i-c):

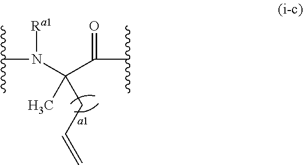

wherein a1 is an integer between 1 and 6, inclusive. In certain embodiments, a1 is 1. In certain embodiments, a1 is 2. In certain embodiments, a1 is 3. In certain embodiments, a1 is 4. In certain embodiments, a1 is 5. In certain embodiments, a1 is 6.

In certain embodiments of formula (ii-a), wherein L₁ and L₂ are each independently C₁₋₆alkylene, provided is an amino acid of Formula (ii-b):

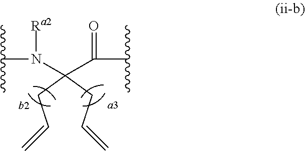

wherein each instance of b2 and a3 is independently an integer between 1 and 6 inclusive. In certain embodiments, each instance of b2 and a3 is 1. In certain embodiments, each instance of b2 and a3 is 2. In certain embodiments, each instance of b2 and a3 is 3. In certain embodiments, each instance of b2 and a3 is 4. In certain embodiments, each instance of b2 and a3 is 5. In certain embodiments, each instance of b2 and a3 is 6.

In certain embodiments, the amino acid of Formula (i) is selected from the group consisting of:

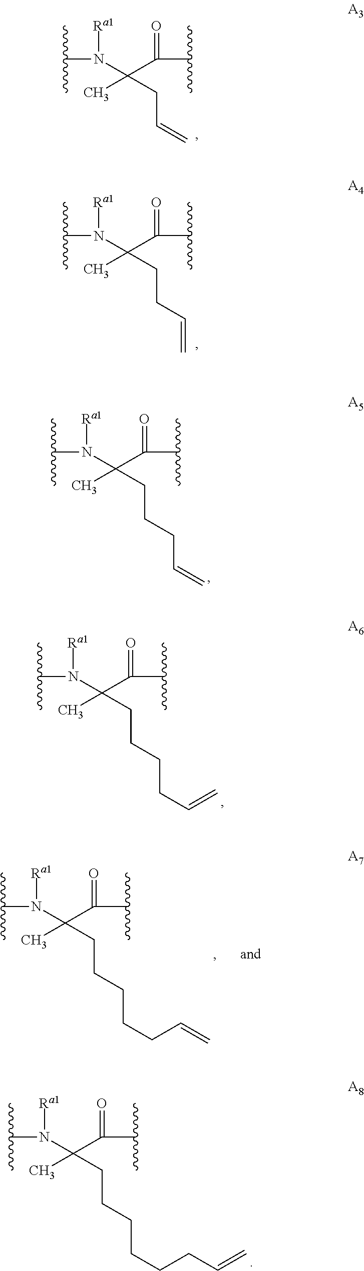

In certain embodiments, at least one instance of the amino acid of Formula (i) is A₅. In certain embodiments, at least one of the amino acid of Formula (i) is A₈.

In certain embodiments, the alpha carbon of the amino acid of Formula (i) is in the (S) configuration. In certain embodiments, the alpha carbon of the amino acid of Formula (i) is in the (R) configuration.

In certain embodiments, the amino acid of Formula (i) is selected from the group consisting of:

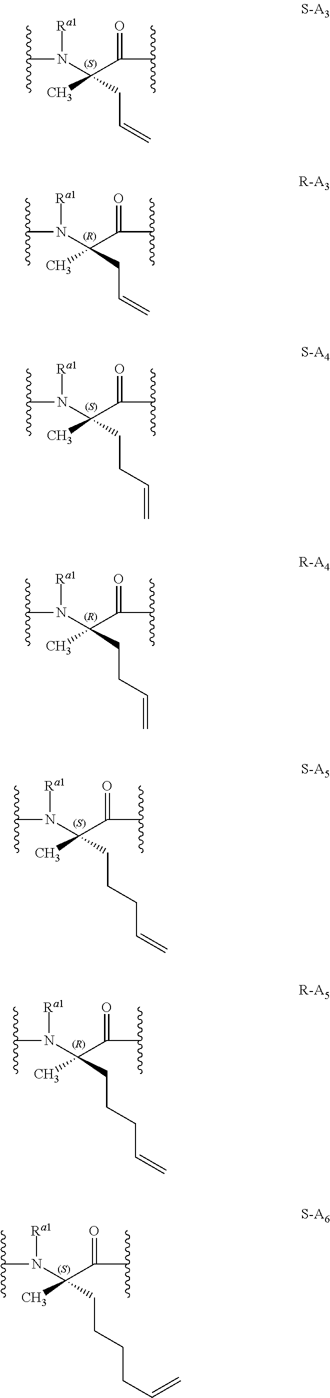

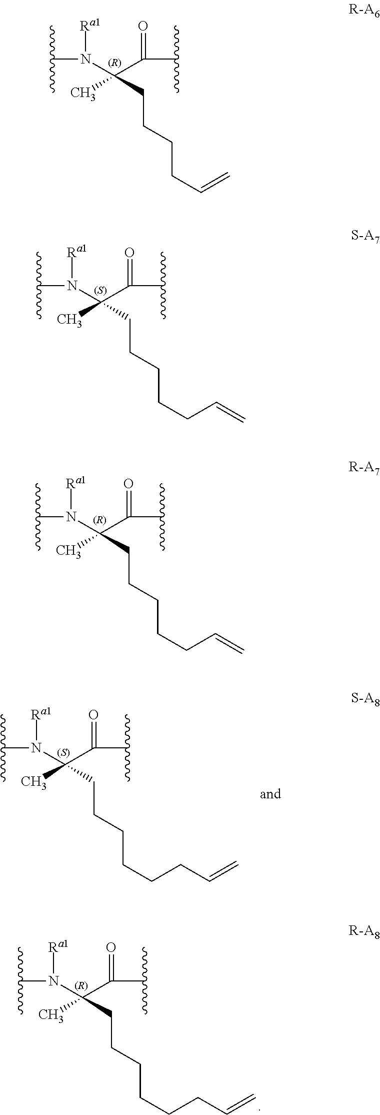

In certain embodiments, at least one of the amino acid of Formula (i) is S-A₅ (also referred to herein as S₅). In certain embodiments, at least one instance of the amino acid of Formula (i) is S-A₈ (also referred to herein as S₈). In certain embodiments, at least one of the amino acid of Formula (i) is R-A₅ (also referred to herein as R₅). In certain embodiments, at least one instance of the amino acid of Formula (i) is R-A₈ (also referred to herein as R₈).

Exemplary amino acids of Formula (ii) include, but are not limited to,

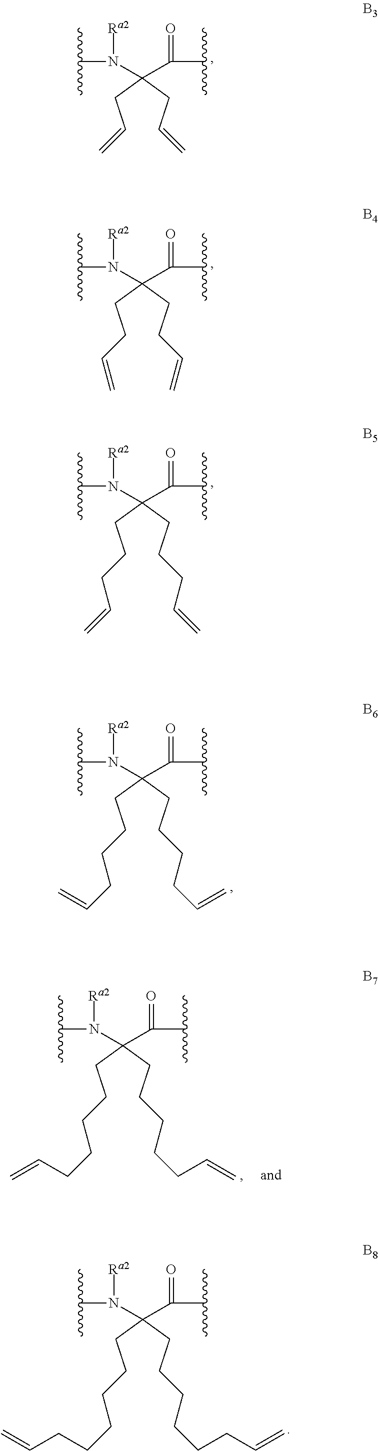

In certain embodiments, each instance of the amino acid of Formula (ii) is B₅.

In certain embodiments, at least one instance of the amino acid of Formula (i) is A₅ and each instance of the amino acid of Formula (ii) is B₅. In certain embodiments, at least one instance of the amino acid of Formula (i) is S₅ and each instance of the amino acid of Formula (ii) is B₅. In certain embodiments, at least one instance of the amino acid of Formula (i) is R₅ and each instance of the amino acid of Formula (ii) is B₅.

In certain embodiments, at least one instance of the amino acid of Formula (i) is A₈ and each instance of the amino acid of Formula (ii) is B₅. In certain embodiments, at least one instance of the amino acid of Formula (i) is S₈ and each instance of the amino acid of Formula (ii) is B₅. In certain embodiments, at least one instance of the amino acid of Formula (i) is R₈ and each instance of the amino acid of Formula (ii) is B₅.

In certain embodiments of Formula (iii) and (iv), wherein q4, q5, and q6 are 0, provided are amino acids of Formula (i-a) and (ii-a):

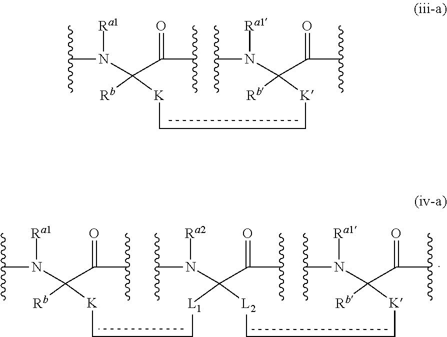

In certain embodiments of Formula (iii-a) or (iv-a), wherein R^{b} is methyl, provided is an amino acids of Formula (iii-b) or (iv-b):

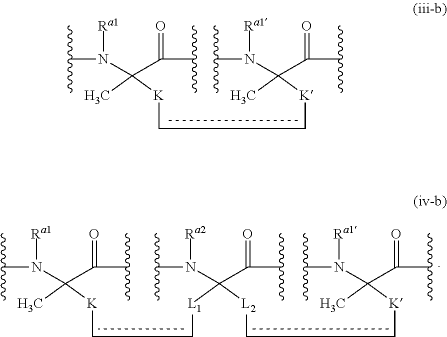

In certain embodiments of Formula (iii-b), wherein K and K′ are C₁₋₆alkylene and [symbol] corresponds to a double bond, provided is a staple of Formula (iii-c):

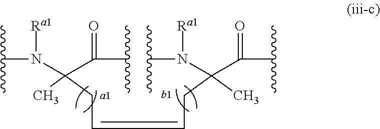

wherein a1 and b1 are an integer between 1 and 6, inclusive. In certain embodiments, both a1 and b1 are 1. In certain embodiments, both a1 and b1 are 2. In certain embodiments, both a1 and b1 are 3. In certain embodiments, both a1 and b1 are 4. In certain embodiments, both a1 and b1 are 5. In certain embodiments, both a1 and b1 are 6. When both a1 and b1 are 3, the amino acids are referred to as stapled A₅-A₅. When both a1 and b1 are 6, the amino acids are referred to as stapled A₈-A₈. When a1 is 3 and b1 is 6, the amino acids are referred to as stapled A₅-A₈. When a1 is 6 and b1 is 3, the amino acids are referred to as stapled A₈-A₅.

In certain embodiments of Formula (iv-b), wherein K, K′, L₁, and L₂ are independently C₁₋₆alkylene and [symbol] corresponds to a double bond, provided is a stitch of Formula (iv-c):

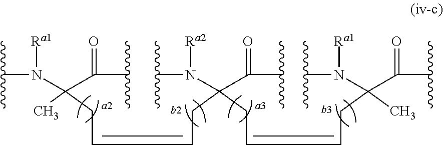

wherein each instance of a2, b2, a3, and b3 is independently an integer between 1 and 6 inclusive. In certain embodiments, each instance of a2, b2, a3, and b3 are 1. In certain embodiments, each instance of a2, b2, a3, and b3 are 2. In certain embodiments, each instance of a2, b2, a3, and b3 are 3. In certain embodiments, each instance of a2, b2, a3, and b3 are 4. In certain embodiments, each instance of a2, b2, a3, and b3 are 5. In certain embodiments, each instance of a2, b2, a3, and b3 are 6. When each instance of a2, b2, a3, and b3 is 3, the amino acids are referred to as stapled A₅-B₅-A₅. When each instance of a2, b2, a3, and b3 is 6, the amino acids are referred to as stapled A₈-B₈-A₈. When each instance of a2, b2, and a3 are 3 and b3 is 6, the amino acids are referred to as stapled A₅-B₅-A₈. When each instance of a2, b2, and a3 are 6 and b3 is 3, the amino acids are referred to as stapled A₈-B₈-A₅.

### Pro-Lock

In certain further embodiments, any of the above referenced precursor polypeptides comprise an amino acid of Formula (v) as a replacement of or in addition to an amino acid of Formula (i):

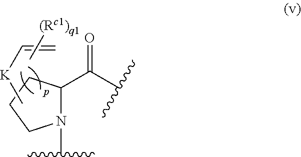

wherein p is 1 or 2, and K, R^{c1}, and q1 are as defined herein.

In certain further embodiments, an amino acid of Formula (v) is of the Formula (v-a) or (v-b):

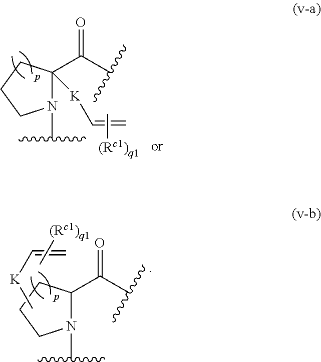

In certain embodiments, the precursor polypeptide comprising an amino acid of Formula (v) and (i), upon contact with a ring closing metathesis catalyst, generates a stapled polypeptide of Formula (vi):

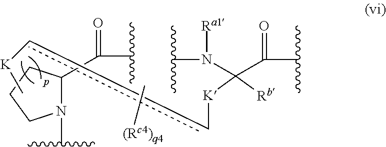

wherein p is 1 or 2, and K, K′, R^{b′}, R^{a1′}, R^{c4}, and q4 are as defined herein.

In certain further embodiments, the staple of Formula (vi) is of the Formula (vi-a) or (vi-b):

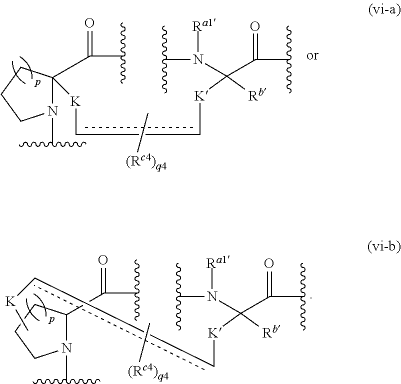

In certain embodiments, the precursor polypeptide comprising an amino acid of Formula (v), (ii), and (i), upon contact with a ring closing metathesis catalyst, generates a stitched polypeptide of Formula (vii):

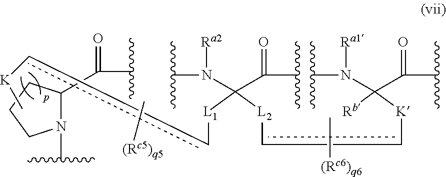

wherein p is 1 or 2, and L₁, L₂, K, K′, R^{b′}, R^{a1′}, R^{a2}, R^{c5}, R^{c6}, q5 and q6 are as defined herein.

In certain further embodiments, the stitch of Formula (vii) is of the Formula (vii-a) or (vii-b):

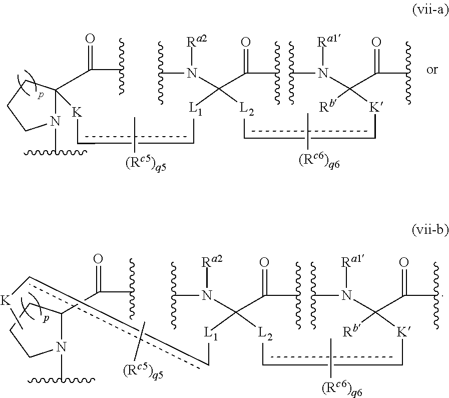

In certain embodiments, the “pro-lock” motif is located at the N-terminus of the polypeptide.

### Dimers or Oligomers

In certain further embodiments, any of the inventive polypeptides such as polypeptides of Formula (I) or (II) are used to form covalent dimers or oligomers. Such dimers or oligomers may have increased affinity and/or potency toward IR. In certain embodiments, the invention provides homodimers of polypeptides of Formula (I) or (II). In certain embodiments, the invention provides heterodimers of polypeptides of Formula (I) or (II). In certain embodiments, the invention provides oligomers of polypeptides of Formula (I) or (II). In certain embodiments, the provided covalent dimers or oligomers have improved agonistic activity toward IR.

The provided dimer has the IR polypeptide monomers linked by a covalent linker (e.g., PEG or amino acids). The provided dimer has the IR polypeptide monomers linked by a covalent linker at the N-terminus of both monomers. The provided dimer has the IR polypeptide monomers linked by a covalent linker at the C-terminus of both monomers. The provided dimer has the IR polypeptide monomers linked by a covalent linker at the N-terminus of one monomer and C-terminus of another monomer.

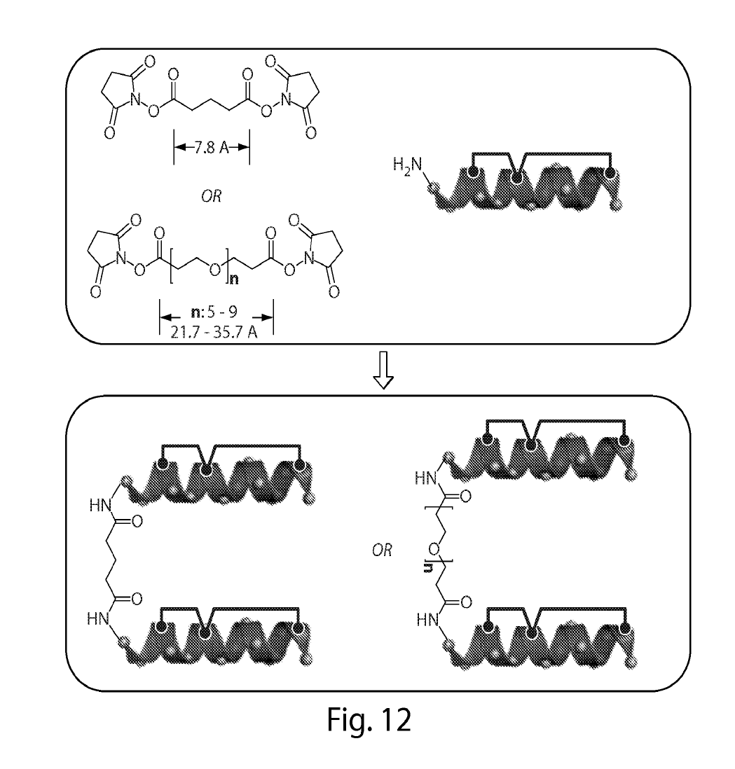

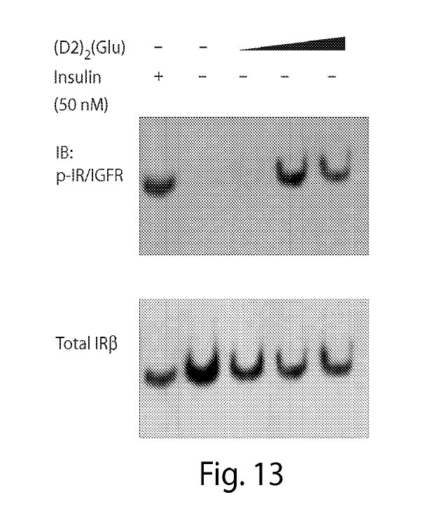

In certain embodiments, the provided dimer is formed by any conjugation method known in the art, for example, native chemical ligation or through a reaction of succinimide with the N-terminal of the IR polypeptide monomer. In certain embodiments, the polypeptide monomers react with bis-succinimide having a spacer such as glutaric acid (Glu), PEG5, or PEG9 to form homodimers or heterodimers. In certain embodiments, IR polypeptide SIRB-B5 reacts with bis-succinimide having a spacer such as glutaric acid (Glu), PEG5, or PEG9 to form homodimers. In certain embodiments, IR polypeptide IRB-D2 reacts with bis-succinimide having a spacer such as glutaric acid (Glu), PEG5, or PEG9 to form homodimers (see FIGS. 12 and 13). In certain embodiments, the provided dimer is one of the following: (SIRB-B5)₂Glu, (SIRB-B5)₂PEG5, (SIRB-B5)₂PEG9, (SIRB-D2)₂Glu, (SIRB-D2)₂PEG5, (SIRB-D2)₂PEG9, wherein Glu (glutaric acid), PEG5, and PEG9 are the linker between the dimers.

### Pharmaceutical Compositions

The present disclosure provides pharmaceutical compositions comprising a stabilized (stitched or stapled) polypeptide as described herein and, optionally, a pharmaceutically acceptable excipient. For the purposes of the present disclosure, the phrase “active ingredient” generally refers to a stabilized polypeptide as described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

The formulations of the pharmaceutical compositions described herein may be prepared by any method known or hereafter developed in the art of pharmacology. In general, such preparatory methods include the step of bringing the active ingredient into association with a carrier and/or one or more other accessory ingredients, and then, if necessary and/or desirable, shaping and/or packaging the product into a desired single- or multi-dose unit.

A pharmaceutical composition may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

The relative amounts of the active ingredient, the pharmaceutically acceptable excipient, and/or any additional ingredients in a pharmaceutical composition of the disclosure will vary, depending upon the identity, size, and/or condition of the subject treated and further depending upon the route by which the composition is to be administered. By way of example, the composition may comprise between 0.1% and 100% (w/w) active ingredient.

Pharmaceutical compositions may comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006) discloses various carriers used in formulating pharmaceutical compositions and known techniques for the preparation thereof. Except insofar as any conventional carrier medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g., polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g., Cremophor), polyoxyethylene ethers, (e.g., polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the polypeptides of the disclosure are mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polethylene glycols and the like.

The active ingredients can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a polypeptide of the disclosure may include ointments, pastes, creams, lotions, gels, powders, solutions, sprays, inhalants and/or patches. Generally, the active component is admixed under sterile conditions with a pharmaceutically acceptable carrier and/or any needed preservatives and/or buffers as may be required. Additionally, the present disclosure contemplates the use of transdermal patches, which often have the added advantage of providing controlled delivery of an active ingredient to the body. Such dosage forms may be prepared, for example, by dissolving and/or dispensing the active ingredient in the proper medium. Alternatively or additionally, the rate may be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

General considerations in the formulation and/or manufacture of pharmaceutical agents may be found, for example, in Remington: The Science and Practice of Pharmacy 21^{st} ed., Lippincott Williams & Wilkins, 2005.

### Methods of Use and Treatment

As generally described herein, in one aspect, provided is a method of treating a diabetic condition or complication thereof comprising administering to a subject in need thereof an effective amount of a stabilized (stapled or stitched) polypeptide as described herein.

As used herein, a “diabetic condition” refers to diabetes and pre-diabetes.

A “subject” to which administration is contemplated includes, but is not limited to, humans (i.e., a male or female of any age group, e.g., a pediatric subject (e.g, infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cats, and/or dogs. In certain embodiments, the animal is a mammal. The animal may be a male or female and at any stage of development. A non-human animal may be a transgenic animal.

“Treat,” “treating” and “treatment” contemplate an action that occurs while a subject is suffering from a diabetic condition or complication and that reduces the severity of the condition or complication or retards or slows the progression of the diabetic condition or complication (“therapeutic treatment”), and also contemplates an action that occurs before a subject begins to suffer from the diabetic condition or complication (e.g., the subject is diagnosed as pre-diabetic) and that inhibits or reduces the severity of the onset of the diabetic condition or complication (“prophylactic treatment”).

The terms “administer,” “administering,” or “administration,” as used herein refers to implanting, absorbing, ingesting, injecting, or inhaling an agent.

An “agent” refers to any therapeutic agent, and includes a stapled or stitched polypeptide as described herein.

An “effective amount” of an agent refers to an amount sufficient to elicit the desired biological response, i.e., treating the diabetic condition or complication. As will be appreciated by those of ordinary skill in this art, the effective amount of an agent may vary depending on such factors as the desired biological endpoint, the pharmacokinetics of the agent, the diabetic condition or complication being treated, the mode of administration, and the age and health of the subject. An effective amount encompasses therapeutic and prophylactic treatment. For example, in treating a diabetic condition or complication, an effective amount of an agent may, for example, reduce, prevent, or delay the onset, of any one of the following symptoms: reduce fasting plasma glucose level [typical diabetic level is ≧7.0 mmol/l (126 mg/dl); typical prediabetic range is 6.1 to 6.9 mmol/1]; reduce plasma glucose [typical diabetic level is ≧11.1 mmol/l (200 mg/dL) two hours after a 75 g oral glucose load as in a glucose tolerance test]; reduce symptoms of hyperglycemia and casual plasma glucose [typical diabetic level is ≧11.1 mmol/l (200 mg/dl)]; reduce levels of glycated hemoglobin (Hb A1C) [typical diabetic level is ≧6.5%]. Subjects with fasting glucose levels from 110 to 125 mg/dl (6.1 to 6.9 mmol/1) are considered to have impaired fasting glucose. Subjects with plasma glucose at or above 140 mg/dL (7.8 mmol/L), but not over 200 mg/dL (11.1 mmol/L), two hours after a 75 g oral glucose load are considered to have impaired glucose tolerance. Of these two pre-diabetic states, the latter in particular is a major risk factor for progression to full-blown diabetes mellitus, as well as cardiovascular disease.

A “therapeutically effective amount” of an agent is an amount sufficient to provide a therapeutic benefit in the treatment of the diabetic condition or complication or to delay or minimize one or more symptoms associated with the diabetic condition or complication. A therapeutically effective amount of an agent means an amount of therapeutic agent, alone or in combination with other therapies, which provides a therapeutic benefit in the treatment of the diabetic condition or complication. The term “therapeutically effective amount” can encompass an amount that improves overall therapy, reduces or avoids symptoms or causes of the diabetic condition or complication, or enhances the therapeutic efficacy of another therapeutic agent.

A “prophylactically effective amount” of an agent is an amount sufficient to prevent a diabetic condition or complication, or one or more symptoms associated with the diabetic condition or complication, or to prevent its recurrence. A prophylactically effective amount of an agent means an amount of a therapeutic agent, alone or in combination with other agents, which provides a prophylactic benefit in the prevention of the diabetic condition or complication. The term “prophylactically effective amount” can encompass an amount that improves overall prophylaxis or enhances the prophylactic efficacy of another prophylactic agent.

Diabetes refers to a group of metabolic diseases in which a person has high blood sugar, either because the body does not produce enough insulin, or because cells do not respond to the insulin that is produced. This high blood sugar produces the classical symptoms of polyuria (frequent urination), polydipsia (increased thirst) and polyphagia (increased hunger).

There are several types of diabetes. Type I diabetes results from the body's failure to produce insulin, and presently requires the person to inject insulin or wear an insulin pump. Type 2 diabetes results from insulin resistance a condition in which cells fail to use insulin properly, sometimes combined with an absolute insulin deficiency. Gestational diabetes occurs when pregnant women without a previous diagnosis of diabetes develop a high blood glucose level. Other forms of diabetes include congenital diabetes, which is due to genetic defects of insulin secretion, cystic fibrosis-related diabetes, steroid diabetes induced by high doses of glucocorticoids, and several forms of monogenic diabetes, e.g., mature onset diabetes of the young (e.g., MODY 1, 2, 3, 4, 5, 6, 7, 8, 9, or 10). Pre-diabetes indicates a condition that occurs when a person's blood glucose levels are higher than normal but not high enough for a diagnosis of diabetes.

All forms of diabetes increase the risk of long-term complications. These typically develop after many years, but may be the first symptom in those who have otherwise not received a diagnosis before that time. The major long-term complications relate to damage to blood vessels. Diabetes doubles the risk of cardiovascular disease and macrovascular diseases such as ischemic heart disease (angina, myocardial infarction), stroke, and peripheral vascular disease. Diabetes also causes microvascular complications, e.g., damage to the small blood vessels. Diabetic retinopathy, which affects blood vessel formation in the retina of the eye, can lead to visual symptoms, reduced vision, and potentially blindness. Diabetic nephropathy, the impact of diabetes on the kidneys, can lead to scarring changes in the kidney tissue, loss of small or progressively larger amounts of protein in the urine, and eventually chronic kidney disease requiring dialysis. Diabetic neuropathy is the impact of diabetes on the nervous system, most commonly causing numbness, tingling and pain in the feet and also increasing the risk of skin damage due to altered sensation. Together with vascular disease in the legs, neuropathy contributes to the risk of diabetes-related foot problems, e.g., diabetic foot ulcers, that can be difficult to treat and occasionally require amputation.

The stabilized polypeptide may be administered using any amount and any route of administration effective for the treatment of the diabetic condition or complication. The exact amount required will vary from subject to subject, depending on the species, age, and general condition of the subject, the severity of the infection, the particular composition, its mode of administration, its mode of activity, and the like.

The stabilized polypeptide is typically formulated in dosage unit form for ease of administration and uniformity of dosage. It will be understood, however, that the total daily usage of the pharmaceutical compositions will be decided by the attending physician within the scope of sound medical judgment. The specific effective dose level for any particular subject will depend upon a variety of factors including the disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The stabilized polypeptide may be administered by any route. In some embodiments, the stabilized polypeptide may be administered by a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general the most appropriate route of administration will depend upon a variety of factors including the nature of the stabilized polypeptide (e.g., its stability in the environment of the gastrointestinal tract), and/or the condition of the subject (e.g., whether the subject is able to tolerate oral administration). The disclosure embraces the delivery of the pharmaceutical compositions as described herein by any appropriate route taking into consideration likely advances in the sciences of drug delivery.

In certain embodiments, the stabilized polypeptide may be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

In certain embodiments, the method comprises administering the stabilized polypeptide as the sole therapeutic agent, but in certain embodiments, the method comprises administering the stabilized polypeptide in combination with another therapeutic agent, such as insulin and/or another anti-diabetic agent. The particular combination will take into account compatibility of the therapeutics and/or procedures and the desired therapeutic effect to be achieved. By “in combination with,” it is not intended to imply that the agents must be administered at the same time and/or formulated for delivery together, although these methods of delivery are within the scope of the disclosure. The agents can be administered concurrently with, prior to, or subsequent to, one or more other desired therapeutics or medical procedures. In general, each agent will be administered at a dose and/or on a time schedule determined for that agent. Additionally, the disclosure encompasses the delivery of the stabilized polypeptide in combination with agents that may improve their bioavailability, reduce and/or modify their metabolism, inhibit their excretion, and/or modify their distribution within the body. In will further be appreciated that the agents utilized in this combination may be administered together in a single pharmaceutical composition or administered separately in different pharmaceutical compositions. In general, it is expected that agents utilized in combination be utilized at levels that do not exceed the levels at which they are utilized individually. In some embodiments, the levels utilized in combination will be lower than those utilized individually.

Insulin is usually given subcutaneously, either by injection or by an insulin pump.

In acute-care settings, insulin may also be given intravenously. In general, there are three types of insulin, characterized by the rate which they are metabolized by the body. They are rapid acting insulins, intermediate acting insulins and long acting insulins. Examples of rapid acting insulins include regular insulin (Humulin R, Novolin R), insulin lispro (Humalog), insulin aspart (Novolog), insulin glulisine (Apidra), and prompt insulin zinc (Semilente, Slightly slower acting). Examples of intermediate acting insulins include isophane insulin, neutral protamine Hagedorn (NPH) (Humulin N, Novolin N), and insulin zinc (Lente). Examples of long acting insulins include extended insulin zinc insulin (Ultralente), insulin glargine (Lantus), and insulin detemir (Levemir).

Other anti-diabetic agents, typically given orally, include, but are not limited to, sulfonylureas (e.g., tolbutamide, acetohexamide, tolazamide, chlorpropamide, glyburide, glimepiride, glipizide, glucopyramide, gliquidone), biguanides (e.g., metformin, phenformin, buformin), meglitinides (e.g., repaglinide, nateglinide), alpha-glucosidase inhibitors (e.g., acarbose, miglitol, voglibose), and thiazolidinediones (e.g., rosiglitazone, pioglitazone, troglitazone).

Further contemplated are uses of the stabilized polypeptides as research tools, i.e., to probe the activation mechanism of the IR.

### Kits

The disclosure provides a variety of kits comprising one or more of the polypeptides disclosed herein. For example, the disclosure provides a kit comprising a stitched or stapled polypeptide as described herein and instructions for use. A kit may comprise multiple different polypeptides. A kit may comprise any of a number of additional components or reagents in any combination. All of the various combinations are not set forth explicitly but each combination is included in the scope of the disclosure

According to certain embodiments of the disclosure, a kit may include, for example, (i) one or more polypeptides and one or more particular biologically active agents to be delivered; (ii) instructions for administering the polypeptide to a subject in need thereof.

Kits typically include instructions which may, for example, comprise protocols and/or describe conditions for production of the polypeptides, administration of the polypeptides to a subject in need thereof, design of the polypeptides, etc. Kits will generally include one or more vessels or containers so that some or all of the individual components and reagents may be separately housed. Kits may also include a means for enclosing individual containers in relatively close confinement for commercial sale, e.g., a plastic box, in which instructions, packaging materials such as styrofoam, etc., may be enclosed. An identifier, e.g., a bar code, radio frequency identification (ID) tag, etc., may be present in or on the kit or in or one or more of the vessels or containers included in the kit. An identifier can be used, e.g., to uniquely identify the kit for purposes of quality control, inventory control, tracking, movement between workstations, etc.

### EXAMPLES

In order that the invention described herein may be more fully understood, the following examples are set forth. It should be understood that these examples are for illustrative purposes only and are not to be construed as limiting this invention in any manner.

### Design and Synthesis of Stapled Insulin Receptor Binding (SIRB) Peptides

Insulin mimetic peptides that were previously evolved by phage display were assigned to two major sets based on two different binding sites on IR. From these peptides S371 was selected, a prototypical site 1 peptide with the highest affinity to the IR (k_{D}=40 nM), as the lead compound for modification with a hydrocarbon cross-link. See, e.g., Pillutla et al., J Biol Chem (2002) 277:22590-22594. S371 spans 16 residues, a length that renders a comprehensive scanning of the hydrocarbon cross-link, as discussed below, elaborate yet still achievable. Moreover, recent revelation of the α-CT structure shows that S371, having a similar sequence motif, may mimic the α-CT in binding to the L1 domain of the receptor. See, e.g., Smith et al., Proc Natl Acad Sci USA (2010) 107:6771-6776. This discovery further adds to the value of optimization of S371 and elucidation of its bound structure to the receptor

Adding a hydrocarbon crosslink on S371 will impart important properties: first, greater secondary structure that will lead to higher target affinity; and second, resistance to proteolytic degradation, higher bioavailability, and longer serum half-life will improve pharmacokinetics on the therapeutic perspective ability. A final trait that may be afforded by this chemical modification is that the hydrophobic nature and lengthy stretch of the cross-link may may enable participation in Van der Waals interactions with the FnIII-1/FnIII-2 loop at site 2, engaging an interface not accessible to an unmodified site 1 peptide. In order to fully explore the physicochemical space reachable by the hydrocarbon cross-link, we have adopted a comprehensive scanning approach in creating the peptide library.

As seen in FIG. 2, several distinct combinations of α,α-disubstituted amino acids were used to form stapled insulin receptor binding (SIRB) peptides with cross-links of various lengths. Utilizing this design strategy, all possible positions for staple incorporation were sampled, e.g., i, i+3; i, i+4; i, i+7; i, i+4+4; i, i+4+7; i, i+7+4; and i, i+7+7. In each series, “i” starts at the C-terminus of the peptide and moves forth until the other end of the crosslink reaches the N-terminus. The stapled peptides were synthesized by solid phase synthesis using standard Fmoc chemistry. as described previously. See, e.g., Kim et al., Nat Protoc (2011) 6:761-771.

Primary screening for active SIRB peptides

The primary signal relay that initiates at the insulin receptor is phosphorylation of the protein itself and downstream effectors or “nodes”. One of the most commonly assessed nodes in the IR pathway is PKB/Akt1, a growth-promoting, antiapoptotic protein responsible for transmitting the mitogenic and metabolic effects of insulin. See, e.g., Scott et al., Proc Natl Acad Sci USA 95, 7772-7777 (1998); Ueki et al., Mol Cell Biol 20, 8035-46 (2000); Tsuruzoe et al., Mol Cell Biol 21, 26-38 (2001); Ueki, K. et al. Proc Natl Acad Sci USA 99, 419-24 (2002); Chang et al., Molecular Medicine 10, 65-71 (2004). We therefore wished to evaluate the activity of SIRB peptides by measuring the changes in phosphorylation level of Akt1. The first cell line we selected to use for screening is HepG2, human liver cancer cells which express high levels of IR and Akt1. See, e.g., Cox et al., Prostate 69, 33-40 (2009); Duronio, Biochem J 270, 27-32 (1990); Gupta et al., J Cell Biochem 100, 593-607 (2007).

For initial screening, each peptide in the library was tested for both antagonistic and agonistic effects using a sandwich ELISA assay kit (Cell Signaling) by measuring phosphorylation of Akt1 at Ser473 performed on HepG2 cells, a liver cancer cell line exhibiting high endogenous IR expression.

Two measurements were made on each compound: 1) treatment of cells with insulin and peptide together to reveal antagonism on the receptor; 2) treatment with peptide alone to reveal agonistic effects. We first validated the assay with unmodified S371 and established the starting point activity: at 10 μM it demonstrated 20% reduction of the maximal phosphorylation level caused by insulin, and no trace of any increase in phosphorylation on its own. We note that although S371 has a high affinity to IR, it can barely induce downstream effects such as lipogenesis in rat adipocytes, which may reconcile with the result of no increase in downstream Akt phosphorylation. The effect of the peptides on insulin signaling was determined using a sandwich ELISA assay kit (Cell Signaling) designed to measure phosphorylation of Akt Ser473, a downstream target in the insulin signaling pathway. For each compound, cells were treated both in the presence and absence of insulin to reveal either antagonism or agonism, respectively, of insulin signaling.

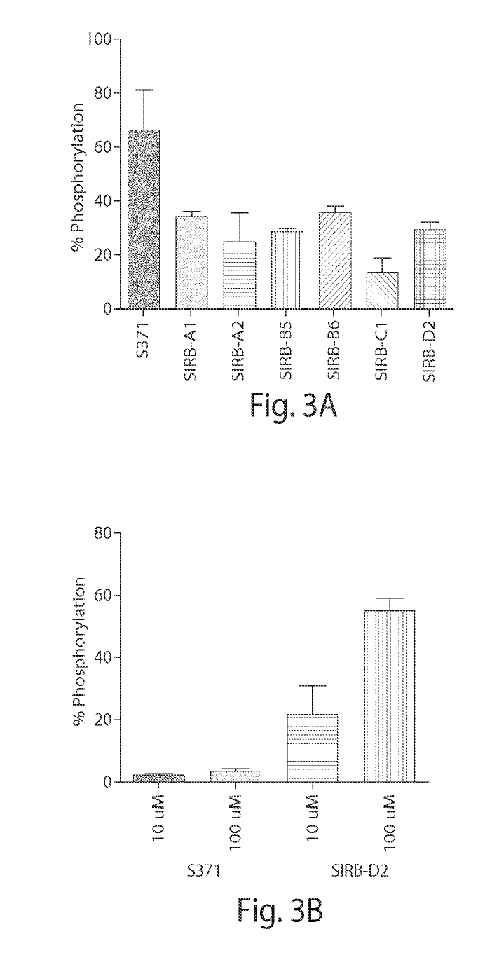

Each cross-link series yielded at least one potential antagonist of insulin signaling (FIG. 3A). Intriguingly, one of these peptides, SIRB-D2, exhibited significant agonistic effects at a concentration of 100 μM, producing 60% of the maximal insulin-induced level of IR activation (FIG. 3B). It was determined that at this high concentration, the SIRB-D2 peptide may form a non-covalent dimer capable of engaging both insulin binding sites on the IR. No similar agonistic effect was observed with any other compound in the library.

The choice of HepG2 cells and Akt1 as a first line of screening was based on the ease of access: the cell line was commercially available and the antibodies of Akt are widely used and very well validated. But we needed a more direct assessment of the IR phosphorylation in order to be confident on the peptides' effects on distal signaling stated above. Furthermore, Akt1 is a node shared by many crisscrossing cellular circuits such as AMPK, mTOR, ErbB/HER pathways. Therefore off-target activity could not be excluded from the possible explanations for the observed effects.

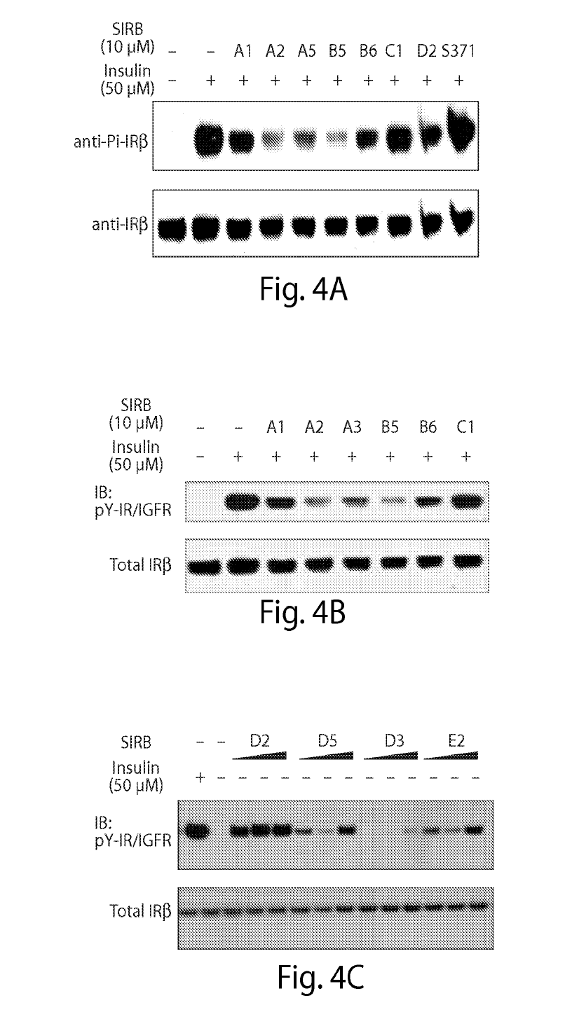

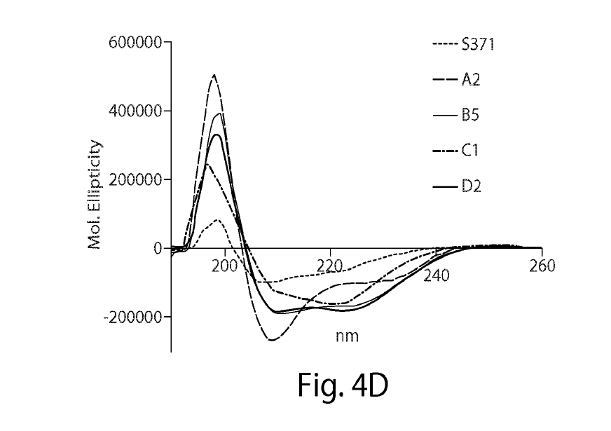

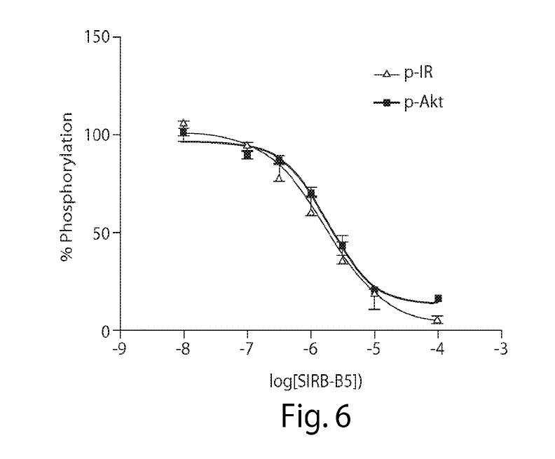

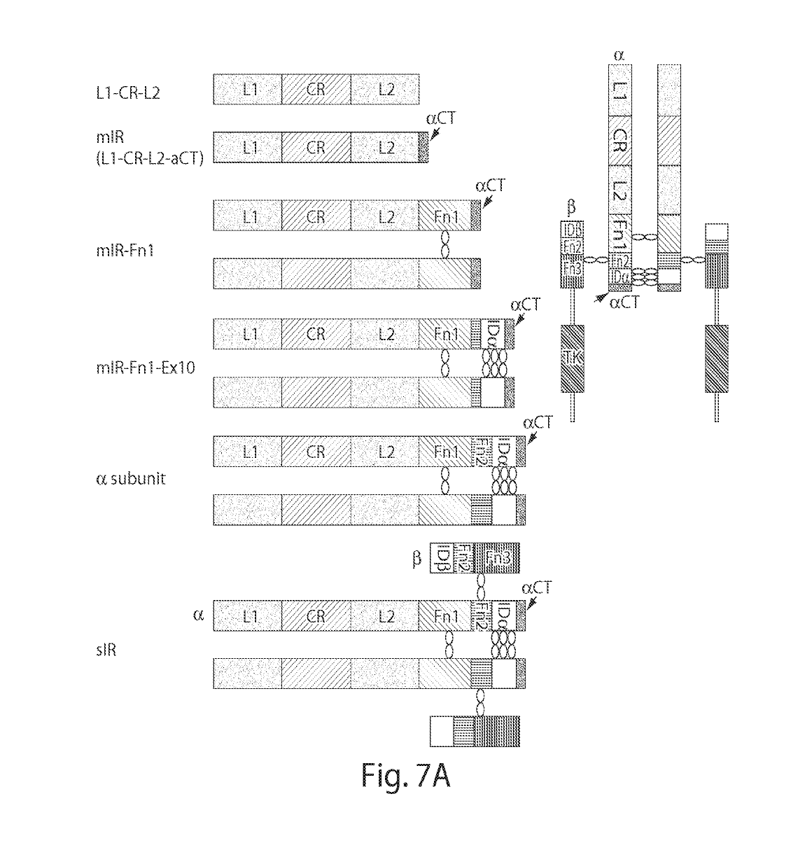

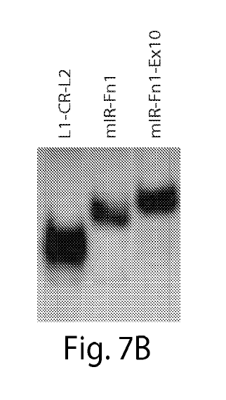

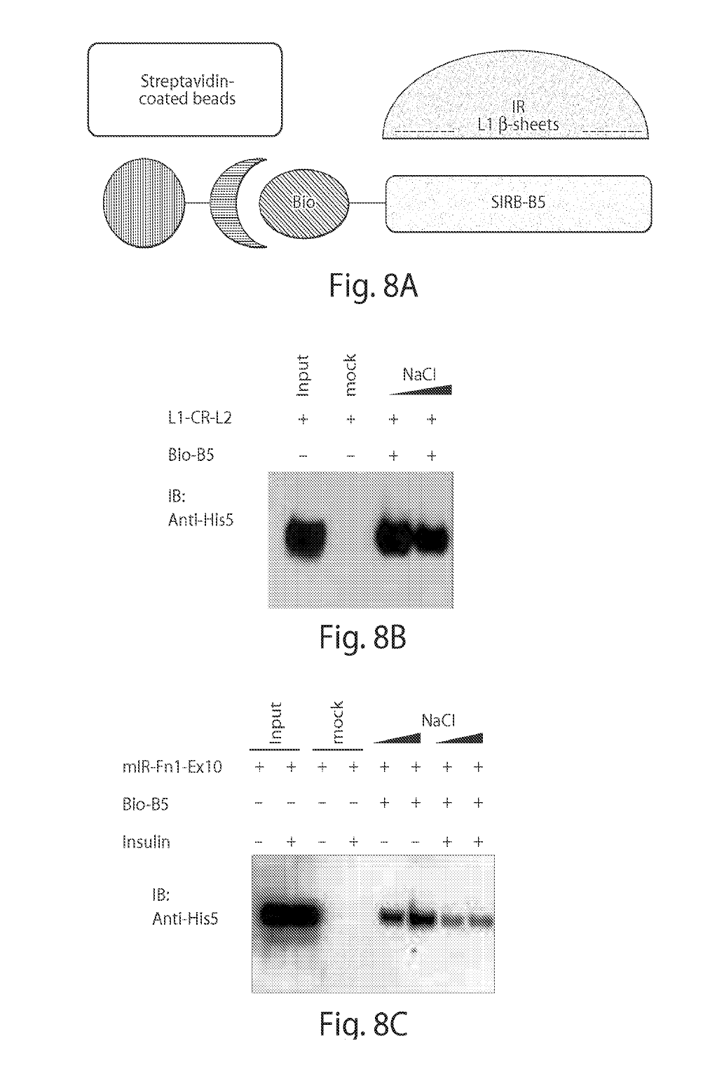

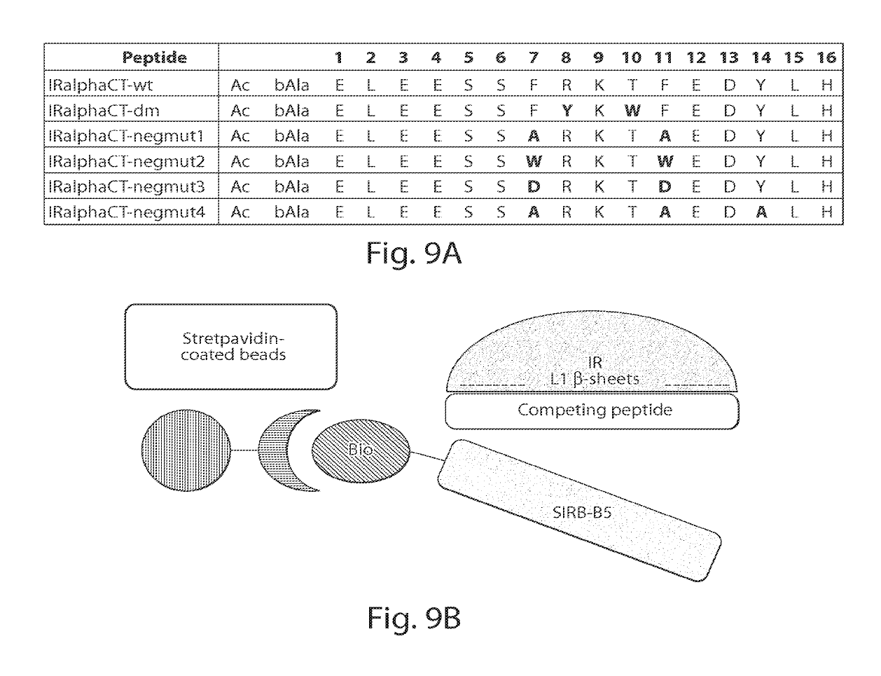

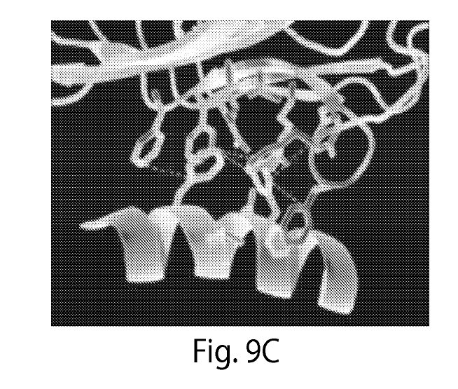

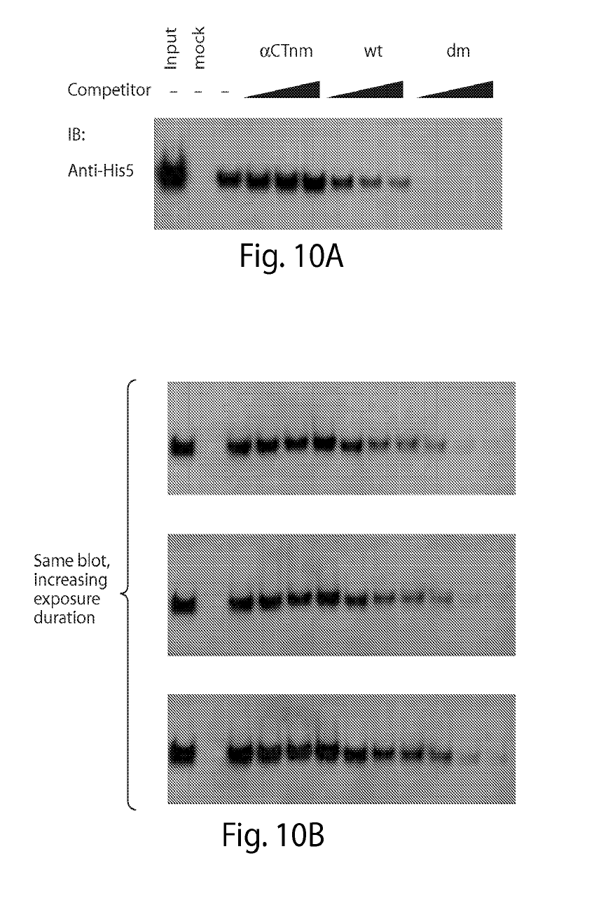

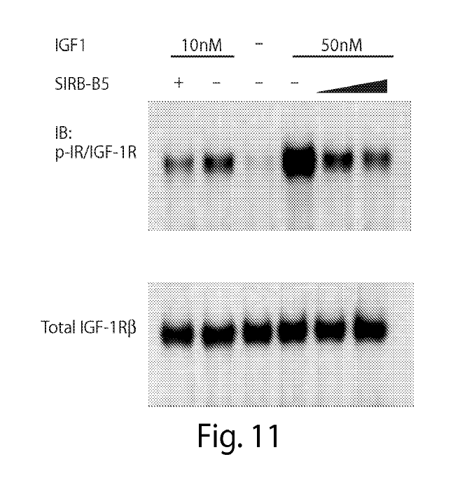

We also quantified the dose-response behavior of the most active antagonist SIRB-B5 on inhibiting insulin-induced autophosphorylation of IR and phosphorylation of Akt with ELISA (FIGS. 4 and 6). For further verification of biological activity, the SIRB peptide library was screened again using IR auto-phosphorylation as an indicator. Upon treatment with stapled peptides identified as insulin antagonists using the initial ELISA-based screen, a reduction was observed in the level of auto-phosphorylation of the IR β-subunit (FIG. 4A). It was observed that SIRB-B5 exerts similar concentration-dependent antagonistic effects on both IR and Akt, with an IC₅₀ value of 1.8 μM. See FIG. 6. Interestingly, at the maximal dose of 100 μM peptide, autophosphorylation of IR is reduced to about 6% of control, whereas Akt phosphorylation is reduced to a lesser extent, 16%. A possible explanation for the differential plateau values is that phosphorylation of Akt1 is controlled by many other pathways as well and thus difficult to abolish completely.

After using these independent measurements of phosphorylation levels as rounds of screening and obtaining consistent results, we felt confident of the peptides identified as either agonists or antagonists of the IR. Investigation of the secondary structure of these stapled peptides using circular dichroism spectroscopy show that the active SIRB peptides have a higher helical content than the unmodified S371 peptide (FIG. 4B). Interestingly, peptide A2, one of the antagonists, shows an intense minimum of molar ellipticity around 208 nm, characteristic of a 310 helix. None of the peptides in other crosslink series exhibits such structural trait. As we move down the lists of series, e.g., going from relatively short and simple to longer and more constrained crosslinks, we observe the general trend of increasing helical character (e.g., compare B5, C1 and D2). Such results are in accordance with our general experience with stapled peptides, whose secondary structure is usually positively correlated with staple length and rigidity.

### Probing the Structural Basis for Interaction of the IR Ectodomain and Active Peptides

To find cross-link positions of the active SIRB peptides, the helical structure of S371 was modeled and docked onto the proposed insulin binding site using the IR-α-CT structure (PDB code: 3LOH) as the template (FIG. 5B).

Surprisingly, the staple hotspots, shown as black spheres in FIG. 5B, clustered on the face of the helix opposite the L1 domain and are in close proximity to the Fn0-Fn1 loop (site 2 of the IR insulin binding site) from the neighboring IR monomer (FIG. 5B). It is clear from the model that there is a hydrophobic interface between the lower surface of S371 and the β-sheet of the L1 domain (site 1 of the IR insulin binding site), and there appears to be space on the upper surface of S371 that may accommodate hydrocarbon staples. Incorporation of a hydrocarbon staple on this upper surface of S371 may increase the interaction between the peptides and the adjacent loop, potentially causing antagonism by locking the receptor in a conformation unproductive for signaling.

FIG. 5A shows the panel of active stapled peptides derived from S371. Sequences of peptide hits are shown grouped in the two categories of antagonists and agonists. Two points should be made here: (1) Although one or two peptides emerge from each type of hydrocarbon staple series as hits, all of them regardless of series or category contain the non-natural amino acids for stapling at converging sites, which if traced back to the original S371 sequence localize to only eight out a total of 16 residues. Structurally speaking, these sites concentrate on a particular face of the helical wheel. (2) Though all hits share said localization of staple positions, only the longest crosslinks seem to confer any agonistic effects (SIRB D2, D5, and E5 are i+4+7 and i+7+7). Coinciding with this result is the trend of increasing helicity discussed above; thus it appears that a more structurally constrained peptide is more prone to antagonize the receptor. Yet longer staples are themselves source of contact surface in addition to simply rigidifying the peptide. The question of whether the staples in the agonist peptides are able to pick up IR residues either nearby on the L1 domain or from the loop in the junction of Fn0/Fn1 domains of the purported Site 2 is both highly relevant and difficult to answer without direct structural information.

### IR Phosphorylation Assay

An SIRB peptide library was synthesized and from this active SIRB peptide were identified by evaluating Akt phosphorylation as an indicator of IR activation. The observed effects were traced back to the phosphorylation of the insulin receptor itself to confirm that the effects seen on Akt are indeed caused by an effect on the IR.

To perform this type of direct assay, a CHO-IR cell line that stably expresses IR (provided by Morris White lab) was treated with SIRB peptides to detect auto-phosphorylation of IRβ Tyr1150/1151 (Cell signaling) by western blot.

It was observed that some active stapled peptides exhibit consistent effects in both the direct and indirect assays, therefore continued screening using the IR phosphorylation assay may be used to assist in the identification of peptides that exhibit the desired on-target effect and may give more hits. Moreover, dose response and time-course experiments may be performed to measure EC50s for IR antagonism and agonism in addition to examining the kinetics with which active SIRB peptides modulate IR signaling using a sandwich ELISA assay kit (Cell Signaling) probing autophosphorylation of IRβ.

On the antagonistic perspective, all of the pre-identified peptides that reduced phosphorylation signals of Akt1 in the presence of insulin also demonstrated similar effects on the IR, and no antagonists of IR alone were found that had not surfaced from the Akt1 ELISA (FIG. 6A) As for agonistic activity in the absence of insulin, there were surprises (FIG. 6B). Peptide SIRB-D2 indeed induced phosphorylation of IR in a dose-response manner, as expected from its effect on Akt1. There were, however, more peptides that are able to induce IR autophosphorylation but had seemingly no observable effect on Akt1: SIRB-D5 and SIRB-E2. This curious discrepancy may be indicative of the peptides' different potencies, i.e. how far down the pathway the signal is transmitted depends on the intensity and duration of the original event at the receptor.

### Clone and Express IR Ectodomain Constructs

The minimized IR (mIR) consisting of the L1-CR-L2 domains exhibits no affinity for insulin except in the presence of the α-CT peptide, which cooperates with the mIR to elicit insulin binding in the low nM range. See, e.g., Kristensen et al., J Biol Chem (1998) 273:17780-17786. Intriguingly, reintroduction of domains involved in the α-α subunit linkage, namely Fn0 and the insertion domain (exon10), into mIR increases the affinity for insulin by approximately 1000 fold (pM range) and restores negative cooperativity. See, e.g., Brandt et al., J Biol Chem (2001) 276:12378-12384; Kristensen et al., J Biol Chem (2002) 277:18340-18345. A series of IR ectodomain constructs may be utilized in binding assays and peptide co-crystallization trials. The expression of these constructs may be tested with transient transfection in CHO Lec3.8.2.1 and 293S GNT I-cell lines, both of which are suitable for the production of crystallization-grade protein preparations due to the lack of complex N- and/or O-glycans. Construct optimization may be needed to produce a construct with high expression.

### Measure the Binding Affinities Between SIRB Peptides and IR Proteins

Well-expressed constructs may be selected for use in biophysical assays, such as SPR and ITC, to measure the affinities of the IR for SIRB peptides. For SPR assays, a Biacore instrument may be used. The IR constructs may be immobilized using either a CM5 or NTA chip. Alternatively, biotin-labeled SIRB peptides can be immobilized using an SA chip and then exposed to the IR constructs as a complementary method of determining the affinity of SIRB peptides for the IR. Binding affinities may be confirmed by ITC. Moreover, SPR and ITC measurements can provide useful insights into the binding mechanism of these peptides, including whether there are conformational changes of the IR upon peptide binding. Even in the absence of high-resolution structural data, this knowledge may assist in optimizing the biological activity of the lead peptides. Additionally, peptides that are shown to induce such changes in the insulin receptor upon binding may be suitable to optimize as potent agonists given that insulin binding is known to induce conformational changes in the IR.

Crystallize SIRB Peptides in Complex with IR Proteins

Well-expressed constructs that contain both insulin-binding sites will be expressed on a large scale by the generation of stable cell lines. Purified protein may be subjected to crystallization in complex with selected peptides. After collecting data, molecular replacement with the published IR ectodomain structure may be used to quickly develop a high-resolution snapshot of the peptide-IR complex. If highly soluble proteins are produced but no crystals are obtained, glycosylation sites may be systematically mutated to produce a more homogenous protein sample for crystallization. Alternatively, to co-crystallizing complex between the peptides and the IR proteins, IR crystals may be soaked with stapled peptides to produce crystals containing the IR-peptide complex. Analyzing the complex structure may result in further data on the binding mode between IR and peptides, the role of hydrocarbon staples in antagonism or agonism, and further optimization of SIRB peptides to increase the affinities or mimic the binding mode of insulin.

### Optimize Active Peptides and Test for Mimetic Activity Both In Vitro and In Vivo

From the perspective of the two-site model of IR activation, a single short SIRB peptide may not be able to engage both insulin binding sites. Therefore, further optimization may produce more potent stapled peptide agonists. Importantly, either antagonist or agonist SIRBs may be modified to produce potent agonists, as it is reported that tethering two IR antagonist peptides may convert them into agonists. However, some antagonist peptides remain antagonistic after linkage, implying dimerization itself is not sufficient for designing agonists. For two given peptides there are four possible ways of tethering. Thus, structural information may serve as a guide for rational design of dimerization. Two approaches may be used for dimerization. In one approach the sequence may be elongated or cross-linking may be utilized to combine a site 2 peptide with a SIRB peptide while maintaining the hydrocarbon staple. Alternatively, a homodimer or heterodimer of active SIRB peptides may be synthetically produced. Either of these approaches may increase both affinity and potency.

### Validate and Characterize Optimized SIRB Peptides

Optimized SIRB peptides are examined and characterized. Additionally, active SIRB peptides are tested for specificity, as the anti-phospho-IR antibody used in the initial screening cannot distinguish phospho-IR from phospho-IGFR. As a result, both CHO-IR and CHO-IGFR cells that stably express IR or IGFR with active SIRB peptides may be treated and a western blot may be performed to detect whether there is a difference in phosphorylation levels. In parallel, using purified IR ectodomain proteins, the binding affinities for full agonists can be easily measured by SPR or ITC.

### Test Mimetic Activity Both In Vitro and In Vivo

The most pronounced effect of insulin signaling in animals is in sugar and fat metabolism. The physiological functions of SIRB peptides may be evaluated by using a glucose uptake assay in 3T3-L1 adipocytes, a well-validated measure of insulin signaling. Next the effect of SIRB peptides on levels of blood glucose and/or lipogenesis in ob/ob or db/db mouse may be evaluated in vivo. Both antagonist and agonist peptides may give consistently sustained, if not amplified, results in downstream physiology in relation to their early effects on protein phosphorylation. Moreover, fluorescein-labeled SIRB peptides may be incubated in mouse serum or injected intravenously into mice to investigate the protease resistance and serum stability in vitro and in vivo. Considering the wealth of data demonstrating that hydrocarbon-stapled peptides are highly resistant to proteolytic degradation, both assays should reveal enhanced stability of SIRB peptides.

### Explore the Activation Mechanism of the IR

A high-resolution structure of the insulin-bound IR would be extremely important for fully understanding IR activation. The unavailability of such a structure is probably due to the moderate affinity of soluble IR ectodomain (sIR) constructs for insulin and the tendency of insulin to dimerize or oligomerize into an inactive form in solution. Although sIR exhibits low affinity for insulin (nM range) and no negative cooperativity, when expressed with a fusion partner at C terminus, the sIR can acquire higher affinity for insulin. For initial screening of the insulin-bound structure, sIR ectodomain constructs may be engineered by fusing with a C-terminal leucine zipper or introducing a disulfide bond that links the β subunits. Additionally, the mIR-Fn0-Ex10 construct, which has been shown to have high affinity (pM range) for insulin, may be tested for crystallization. See, e.g., Brandt et al., J Biol Chem (2001) 276:12378-12384; Kristensen et al., J Biol Chem (2002) 277:18340-18345. Considering that the binding of insulin to either IR-fusion proteins or mIR-Fn0-Ex10 may be unstable due to negative cooperativity, the mIR-Fn0 construct (reported as IR593.CT) with medium affinity for insulin, slow dissociation, and no cooperativity will serve as an alternative for crystal screening. See, e.g., Surinya et al., J Biol Chem (2002) 277:16718-16725. Engineered monomeric insulin may also be used in these crystallization studies. The agonist SIRB peptide obtained may serve as an alternative to insulin in these crystallization trials.

Furthermore, whether the conformation and location of α-CT will change upon insulin binding remains a critical question regarding IR activation. Thus, as a backup to fully illustrate IR activation, all well-expressed IR ectodomain constructs that contain α-CT segments will be subjected to crystallization screening both in the absence and presence of insulin or agonist peptide to elucidate the role of α-CT in insulin activation. Since these constructs would be optimized for previous crystallization, crystallization of these constructs with insulin or agonist peptides should be a readily obtainable and highly informative goal.

### Additional Experimental Methods

### Peptide Synthesis

SIRB Library peptides were synthesized using the standard Fmoc-SPPS protocol and purified on reverse-phase HPLC.

### Cell Culture

HepG2 cells (ATCC) were maintained in complete growth medium consisting of DMEM, 10% fetal bovine serum (FBS) and streptomycin/penicillin. Cells were passaged when reaching greater than 80% confluence (on average 3-4 days) and split 1:6 per passage. CHO-IR cells (from Prof. Morris White at Boston Children's Hospital) were maintained in complete growth medium consisting of Ham's F-12, 10% FBS and streptomycin/penicillin. Cells were passaged when reaching greater than 80% confluence (on average 2-3 days) and split 1:10 per passage.

### Phospho-Akt1 ELISA

Preparation: insulin stock solution at 50 μM (0.3 mg/ml or 30 mg/100 ml). To make stock: dissolved 10 mg in 1 ml 0.01N HCl, heat at 37° C. to dissolve, then added to 32 mL PBS, sterile filtered and stored at −20 C. Working stock would stay in good quality for 1 month at 4° C. HepG2 cells grown to 80% confluent in 10 cm plates or 6-well plates were serum-starved in DMEM (penicillin/streptomycin added) overnight. On the next day, the following solutions were prepared: 50 nM insulin in complete growth medium (from 50 μM stock), 10 μM or 100 μM peptide in complete growth medium (from 10 mM DMSO stock), and vehicle (the highest concentration of DMSO used in peptide samples). Old media was removed and cells were treated with appropriate compounds. For measurement of antagonism, the vehicle was used to create baseline, and the 50 nM insulin solution was used to give maximal amount of signal (100% phosphorylation). Each of the peptides was given in combination with 50 nM insulin. For measurement of agonism, the baseline and maximal level of phosphorylation were obtained the same way. Each of the peptides was given along in the absence of insulin. Cells were incubated at 37° C. under 5% CO₂ for 15 min for antagonism and 30 min for agonism.

At the end of treatment, media was aspirated and cells were washed with ice cold PBS, and subsequently lysed using Lysis Buffer (from 10× Lysis Buffer, Cell Signaling; 1 mM PMSF added prior to use). Cells were incubated in Lysis Buffer plates/wells in the cold room with gentle rocking for 10 min, then subjected to quick sonication. The cell lysate was then centrifuged at 12,000 rpm for 10 min in the cold room. The clarified lysate was collected and aliquots were flash frozen in liquid nitrogen and stored at −80° C. until use.

For the phospho-Akt1 ELISA assay, clarified lysate was first quantified using the standard BCA method, and all samples were adjusted to have the same total protein concentration. All samples were then diluted with Sample Diluent (from phospho-Akt1 S473 ELISA kit Cell Signaling) at 1:1, and added to antibody-coated microwells. Microwells were sealed firmly with tape and incubated at 4° C. overnight.

The following day, wells were drained and washed four times with 1× Wash Buffer. 50 ul detection antibody was added to each well, sealed with tape, and incubated 1 h at room temperature. Contents were discarded and wells were washed with 1× Wash Buffer four times. 50 ul HRP-conjugated secondary antibody was to each well, seale with tape, and incubated for 30 minutes at room temperature. Contents were discarded and wells were washed again four times. Luminol/Enhancer solutions were mixed 1:1, and 50 ul was added to each well. Chemiluminescence was read under RLU mode in the Hewlett-Packard SpectraMax Plate Reader at 425 nM within 1-10 minutes of addition of substrate.

### Immunoblotting of IR Auto-Phosphorylation

CHO-IR cells were seeded in 24-well plates and grown in Ham's F-12, 10% FBS medium to >80% confluent (in ˜48 hours). The cells are starved in Ham's F-12 medium without FBS for two hours prior to experiment.

For agonistic studies, cells were incubated in fresh Ham's F-12 without FBS (negative control and baseline for normalization) or the same medium with 50 nM insulin (positive control and 100% activity for normalization). Experimental wells were incubated in the same medium containing various concentrations of peptide (typically 1 uM, 10 uM, and 100 uM). All cells were incubated at 37° C. for 15 min or 30 min.

For antagonistic studies, the experiment could be done with or without pre-treatment with peptides. In the case without, cells were incubated in fresh Ham's F-12 without FBS (negative control and baseline for normalization) or the same medium with 50 nM insulin (positive control and 100% activity for normalization).

Experimental wells were incubated in the same medium containing various concentrations of peptide (typically 1 uM, 10 uM, and 100 uM) AND 50 nM insulin. All cells were incubated at 37 C for 15 min. In the case with pre-treatment, experimental wells are pre-incubated with various concentration of peptide (typically 1 uM, 10 uM, and 100 uM) at 37° C. for 30 min before insulin (final concentration 50 nM) was added. In the case with pre-treatment, all is the same except the experimental groups were pre-incubated with peptide for 30 min before the addition of insulin. At the end of incubation all medium is removed and cells are washed once with ice-cold PBS. The cells will remain on ice from this point on. Ice-cold lysis buffer is added (50-100 uL/well for 24-well plates).

Composition of lysis buffer: [1] SDS loading buffer, from 4× stock (To make 10 mL of 4× stock: 2.0 ml IM Tris-HCl pH 6.8, 0.8 g SDS, 4.0 ml 100% glycerol, 0.4 ml 14.7 M β-mercaptoethanol, 1.0 ml 0.5 M EDTA, 8 mg bromophenol Blue; sonicate to dissolve); [2] PMSF, 1 uM, from 100× stock in ethanol; [3] Protease inhibitor 1 and phosphatase inhibitors 2 & 3 from Sigma Aldrich, all from 100X; 5% beta-mercaptoethanol; [4] Fill up volume to 1× with TBS, from 10× stock, Mediatech (PMSF, protease inhibitors, and BME were added right before experiment).

Cells are lysed in wells for greater than 5 minutes then collected by cell scrapers or scraping gently with pipet tips. The lysates were boiled for 15 minutes. They could be directly loaded onto SDS-PAGE gel or stored at −80° C.

Western Blot: SDS-PAGE gel is transferred to nitrocellulose or PVDF membrane (PVDF must be primed with methanol before use) for 100 min at 33V in the cold room. The membrane is washed briefly in TBS-T (IX TBS, 0.1% Tween-20) and incubated in blocking buffer (5% BSA in TBS-T, filtered) for 1 h at room temperature. The membrane is washed 2× and incubated with primary antibody (Phospho-IGF-I Receptor β (Tyr1135/1136)/Insulin Receptor β (Tyr1150/1151) (19H7) Rabbit mAb Cell Signaling #3024), 1:300 in 3% BSA in TBS-T) overnight at 4 C. The membrane is washed four times, 5-10 minutes each in TBS-T, then incubated with secondary antibody (Anti-rabbit IgG, HRP-linked Antibody Cell Signaling #7074) 1:3000 in 3% BSA in TBS-T) for 1 h at room temperature. The membrane was washed four times for 5-10 min. The chemiluminescence is developed by applying a mixture of Pico-level substrate and 1% Femto-level substrate (Thermo) to the blot. Subsequently, the blot can be stripped and reblot for total IR (Insulin Receptor β (4B8) Rabbit mAb Cell Signaling #3025) in the same manner.

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims.

Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A polypeptide comprising an alpha-helical segment, wherein the polypeptide binds to the insulin receptor, and wherein the polypeptide comprises at least two cross-linked amino acids of Formula (iii): 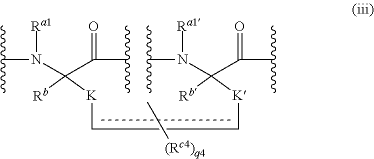
or at least three cross-linked amino acids of Formula (iv): 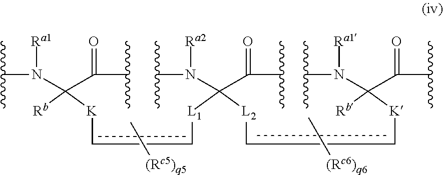
wherein:
each instance of K, K′, L₁, and L₂, is, independently a bond or a group consisting of one or more combinations of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted heterocyclene, substituted and unsubstituted carbocyclene; substituted and unsubstituted arylene; and substituted and unsubstituted heteroarylene;
each instance of R^{a1}, R^{a1′}, and R^{a2} is, independently, hydrogen; substituted or unsubstituted aliphatic; substituted or unsubstituted heteroaliphatic; substituted or unsubstituted aryl; substituted or unsubstituted heteroaryl; acyl; or an amino protecting group;
each instance of R^{b} and R^{b′} is, independently, hydrogen; substituted or unsubstituted aliphatic; substituted or unsubstituted heteroaliphatic; substituted or unsubstituted aryl; or substituted or unsubstituted heteroaryl;
each instance of [symbol] independently represents a single or double bond;
each instance of R^{c4}, R^{c5}, and R^{c6} is independently hydrogen; substituted or unsubstituted aliphatic; substituted or unsubstituted heteroaliphatic; substituted or unsubstituted aryl; substituted or unsubstituted heteroaryl; acyl; substituted or unsubstituted hydroxyl; substituted or unsubstituted thiol; substituted or unsubstituted amino; azido; cyano; isocyano; halo; or nitro; and
each instance of q^{c4}, q^{c5}, and q^{c6} is independently 0, 1, or 2.
or a pharmaceutically acceptable salt thereof.

2. The polypeptide of claim 1, wherein the polypeptide is of Formula (II): 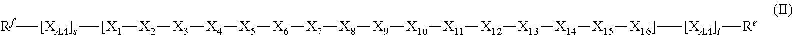
or a pharmaceutically acceptable salt thereof;
wherein:
each [X_{AA}] is independently a natural or unnatural amino acid;
s is 0 or an integer of between 1 to 50, inclusive;
t is 0 or an integer of between 1 to 50, inclusive;
R^{f} is an N-terminal group selected from the group consisting of hydrogen; substituted and unsubstituted aliphatic; substituted and unsubstituted heteroaliphatic; substituted and unsubstituted aryl; substituted and unsubstituted heteroaryl; acyl; a resin; an amino protecting group; and a label optionally joined by a linker, wherein the linker is a group consisting of one or more combinations of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted arylene; substituted and unsubstituted heteroarylene; and acylene;
R^{e} is a C-terminal group selected from the group consisting of hydrogen; substituted and unsubstituted aliphatic; substituted and unsubstituted heteroaliphatic; substituted and unsubstituted aryl; substituted and unsubstituted heteroaryl; —OR^{E}; —N(R^{E})₂; and —SR^{E}, wherein each instance of R^{E} is, independently, hydrogen; substituted or unsubstituted aliphatic; substituted or unsubstituted heteroaliphatic; substituted or unsubstituted aryl; substituted or unsubstituted heteroaryl; acyl; a resin; a protecting group; or two R^{E} groups taken together form an substituted or unsubstituted heterocyclic or substituted or unsubstituted heteroaryl ring;
X₁ is amino acid G or is an amino acid which forms together with another amino acid a staple of Formula (iii);
X₂ is amino acid S or is an amino acid which forms together with another amino acid a staple of Formula (iii);
X₃ is amino acid L;
X₄ is amino acid D;
X₅ is amino acid E, is an amino acid which forms together with another amino acid a staple of Formula (iii), or is an amino acid which forms together with two other amino acids a stitch of Formula (iv);
X₆ is amino acid S, is an amino acid which forms together with another amino acid a staple of Formula (iii), or is an amino acid which forms together with two other amino acids a stitch of Formula (iv);
X₇ is amino acid F;
X₈ is amino acid Y;
X₉ is amino acid D or is an amino acid which forms together with another amino acid a staple of Formula (iii);
X₁₀ is amino acid W;
X₁₁ is amino acid F;
X₁₂ is amino acid E or is an amino acid which forms together with another amino acid a staple of Formula (iii);
X₁₃ is amino acid R or is an amino acid which forms together with another amino acid a staple of Formula (iii);
X₁₄ is amino acid Q;
X₁₅ is amino acid L; and
X₁₆ is amino acid G;
provided that the amino acid sequence comprises at least one staple of Formula (iii) or at least one stitch of Formula (iv).

3. A polypeptide comprising an alpha-helical segment, wherein the polypeptide binds to the insulin receptor, and wherein the polypeptide comprises at least two amino acid moieties of formula (i), and optionally, one amino acid of Formula (ii): 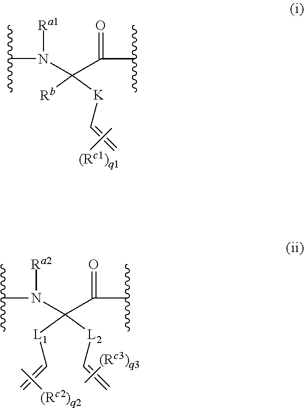
wherein:
each instance of K, L₁, and L₂, is, independently a bond or a group consisting of one or more combinations of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted heterocyclene; substituted and unsubstituted carbocyclene; substituted or unsubstituted arylene; and substituted and unsubstituted heteroarylene;
each instance of R^{a1} and R^{a2} is, independently, hydrogen; substituted or unsubstituted aliphatic; substituted or unsubstituted heteroaliphatic; substituted or unsubstituted aryl; substituted or unsubstituted heteroaryl; acyl; or an amino protecting group;
R^{b} is hydrogen; substituted or unsubstituted aliphatic; substituted or unsubstituted heteroaliphatic; substituted or unsubstituted aryl; substituted or unsubstituted heteroaryl;
each instance of R^{c1}, R^{c2}, and R^{c3} is independently hydrogen; substituted or unsubstituted aliphatic; substituted or unsubstituted heteroaliphatic; substituted or unsubstituted aryl; substituted or unsubstituted heteroaryl; acyl; substituted or unsubstituted hydroxyl; substituted or unsubstituted thiol; substituted or unsubstituted amino; azido; cyano; isocyano; halo; or nitro; and
each instance of q^{c1}, q^{c2}, and q^{c3} is independently 0, 1, or 2;
or a pharmaceutically acceptable salt thereof.

4. The polypeptide of claim 3, wherein the polypeptide is of Formula (I): 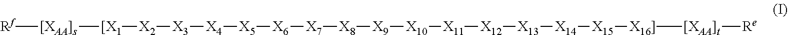
or a pharmaceutically acceptable salt thereof;
wherein:
each [X_{AA}] is independently a natural or unnatural amino acid;
s is 0 or an integer of between 1 and 50, inclusive;
t is 0 or an integer of between 1 and 50, inclusive;
R^{f} is an N-terminal group selected from the group consisting of hydrogen; substituted and unsubstituted aliphatic; substituted and unsubstituted heteroaliphatic; substituted and unsubstituted aryl; substituted and unsubstituted heteroaryl; acyl; a resin; an amino protecting group; and a label optionally joined by a linker, wherein the linker is a group consisting of one or more combinations of substituted and unsubstituted alkylene; substituted and unsubstituted alkenylene; substituted and unsubstituted alkynylene; substituted and unsubstituted heteroalkylene; substituted and unsubstituted heteroalkenylene; substituted and unsubstituted heteroalkynylene; substituted and unsubstituted arylene; substituted and unsubstituted heteroarylene; and acylene;
R^{e} is a C-terminal group selected from the group consisting of hydrogen; substituted and unsubstituted aliphatic; substituted and unsubstituted heteroaliphatic; substituted and unsubstituted aryl; substituted and unsubstituted heteroaryl; —OR^{E}; —N(R^{E})₂; and —SR^{E}; wherein each instance of R^{E} is, independently, hydrogen; substituted or unsubstituted aliphatic; substituted or unsubstituted heteroaliphatic; substituted or unsubstituted aryl; substituted or unsubstituted heteroaryl; acyl; a resin; a protecting group; or two R^{E} groups taken together form an substituted or unsubstituted heterocyclic or substituted or unsubstituted heteroaryl ring;
X₁ is amino acid G or an amino acid of Formula (i);
X₂ is amino acid S or an amino acid of Formula (i);
X₃ is amino acid L;
X₄ is amino acid D;
X₅ is amino acid E, an amino acid of Formula (i), or an amino acid of Formula (ii);
X₆ is amino acid S, an amino acid of Formula (i), or an amino acid of Formula (ii);
X₇ is amino acid F;
X₈ is amino acid Y;
X₉ is amino acid D or an amino acid of Formula (i);
X₁₀ is amino acid W;
X₁₁ is amino acid F;
X₁₂ is amino acid E or an amino acid of Formula (i);
X₁₃ is amino acid R or an amino acid of Formula (i);
X₁₄ is amino acid Q;
X₁₅ is amino acid L; and
X₁₆ is amino acid G;
provided that the amino acid sequence comprises at least two independent occurrences of an amino acid of Formula (i), and/or at least one occurrence of Formula (ii) and two amino acids of Formula (i) peripheral thereto.

5. The polypeptide of any one of claims 1 to 4, wherein K is substituted or unsubstituted C₁₋₆ alkylene.

6. The polypeptide of claims 1 or 2, wherein K is substituted or unsubstituted C₁₋₆ alkylene.

7. The polypeptide of any one of claims 1 to 4, wherein L₁ is substituted or unsubstituted C₁₋₆ alkylene.

8. The polypeptide of any one of claims 1 to 4, wherein L₂ is substituted or unsubstituted C₁₋₆ alkylene.

9. The polypeptide of claims 1 or 2, wherein [symbol] is a double bond.

10. The polypeptide of claims 1 or 2, wherein q^{c4} q^{c5}, and q^{c6} is 0.

11. The polypeptide of claims 3 or 4, wherein q^{c1}, q^{c2}, and q^{c3} is 0.

12. The polypeptide of claims 3 or 4, wherein the amino acid of Formula (i) is selected from the group consisting of:

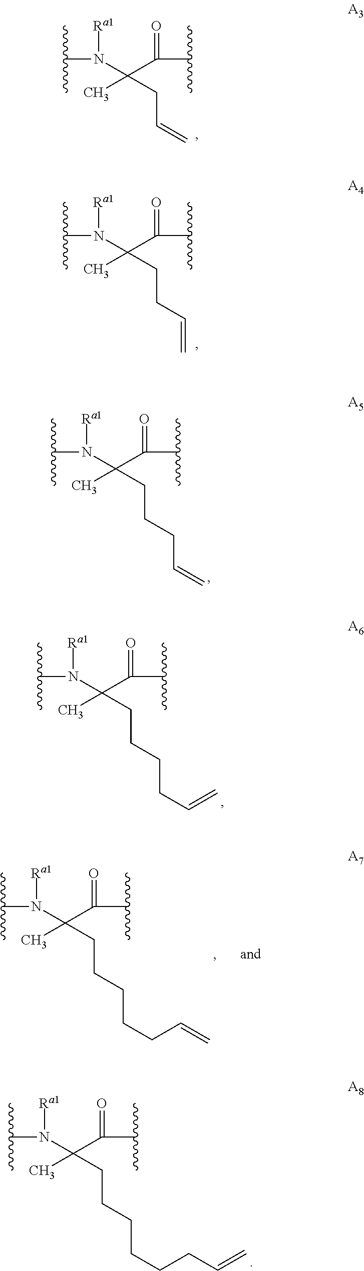

13. The polypeptide of claims 3 or 4, wherein the amino acid of formula (ii) is selected from the group consisting of:

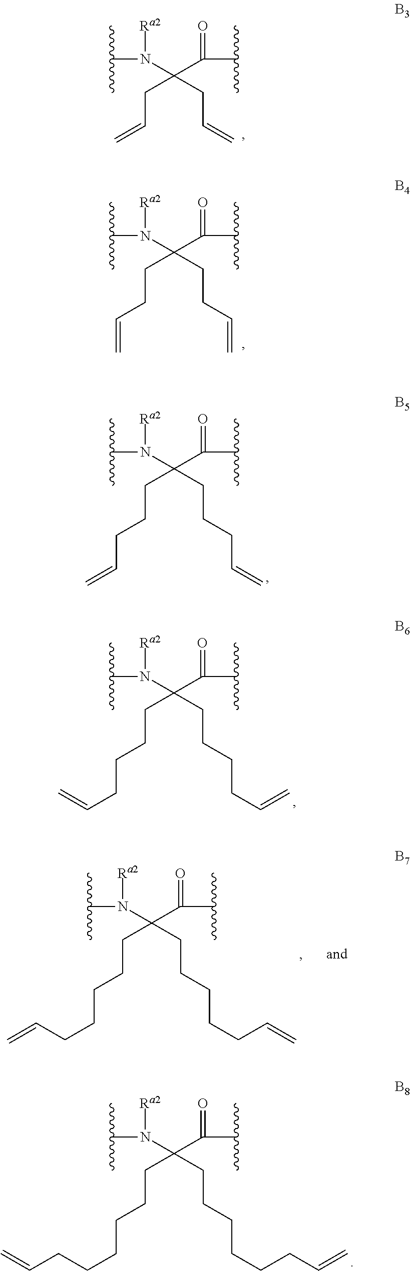

14. The polypeptide of claim 1 or 2, selected from any one of the stapled or stitched polypeptides depicted in FIG. 15.

15. A pharmaceutical composition comprising a polypeptide of claim 1 or 2 or a pharmaceutically acceptable salt thereof and a pharmaceutically acceptable excipient.

16. A method of treating a diabetic condition or a complication thereof comprising administering to a subject in need thereof an effective amount of a polypeptide of claim 1 or 2 or a pharmaceutically acceptable salt thereof.

17. The method of claim 16, wherein the diabetic condition is diabetes or pre-diabetes.

18. The method of claim 16, wherein the diabetes is Type I diabetes, Type 2 diabetes, gestational diabetes, congenital diabetes, cystic fibrosis-related diabetes, steroid diabetes, or monogenic diabetes.

19. The method of claim 16, wherein the complication of the diabetic condition is cardiovascular disease, ischemic heart disease, stroke, peripheral vascular disease, damage to blood vessels, diabetic retinopathy, diabetic nephropathy, chronic kidney disease, diabetic neuropathy, and diabetic foot ulcers.
